SEQUENCE LISTING (142 sequences)

SEQ ID NO: 1 - protein, 7 aa, synthetic construct
TLAAPFK

SEQ ID NO: 2 - protein, 8 aa, synthetic construct
DDDDDDDD

SEQ ID NO: 3 - protein, 7 aa, synthetic construct
LSSRLDA

SEQ ID NO: 4 - protein, 9 aa, synthetic construct
CLSSRLDAC

SEQ ID NO: 5 - protein, 7 aa, synthetic construct
CLPVASC

SEQ ID NO: 6 - protein, 5 aa, synthetic construct
LPVAS

SEQ ID NO: 7 - protein, 7 aa, synthetic construct
ASSLNIA

SEQ ID NO: 8 - skipped

SEQ ID NO: 9 - protein, 4 aa, synthetic construct
STQT

SEQ ID NO: 10 - protein, 7 aa, synthetic construct
HAIYPRH

SEQ ID NO: 11 - skipped

SEQ ID NO: 12 - protein, 7 aa, synthetic construct
RTIGPSV

SEQ ID NO: 13 - protein, 9 aa, synthetic construct
CRTIGPSVC

SEQ ID NO: 14 - protein, 11 aa, synthetic construct
SITLVKSTQTV

SEQ ID NO: 15 - protein, 10 aa, synthetic construct
TILSRSTQTG

SEQ ID NO: 16 - protein, 7 aa, synthetic construct
QAVRTSL

SEQ ID NO: 17 - protein, 7 aa, synthetic construct
QAVRTSH

SEQ ID NO: 18-19 - skipped (2 entries)

SEQ ID NO: 20 - protein, 7 aa, synthetic construct
TLAVPFK

SEQ ID NO: 21 - skipped

SEQ ID NO: 22 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQAAASNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQAAASNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVAGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 744 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDDDDDDDDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLSSRLDAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 745 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLSSRLDACGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLPVASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLPVASCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASSLNIAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHAIYPRHGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRTIGPSVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 745 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCRTIGPSVCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 747 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSITLVKSTQTVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTILSRSTQTGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 753 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTTILSRSTQTGAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 754 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTSITLVKSTQTVAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 700 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPE

SEQ ID NO: 49 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSAGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52-58 - skipped (7 entries)

SEQ ID NO: 59 - protein, 735 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 735 aa, synthetic construct [sequence omitted]

SEQ ID NO: 61 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 62 - protein, 732 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGSLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYQQNSLLWAPDAAGKYTEPRAIGTRYLTHHL

SEQ ID NO: 63 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 64 - protein, 735 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 65 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 66 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 67 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 70 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 71 - protein, 797 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 72 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 73 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 74 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 75 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 76 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 77 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 78 - protein, 736 aa, synthetic construct; identical to SEQ ID NO: 61

SEQ ID NO: 79 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 80 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 81 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 82 - protein, 733 aa, synthetic construct
MAADGYLPDWLEDNLSEGIREWWDLKPGAPKPKANQQKQDDGRGLVLPGYEYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFETAYNFEKVPFHSMYAHSQSLDGLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPAYPATTFTAARVDSFITQYSTGQVAVQIEWEIEKERSKRWNPEVQFTSNCGNQSSMLWAPDTTGKYTEPRVIGSRYLTNHL

SEQ ID NO: 83 - protein, 735 aa, synthetic construct; identical to SEQ ID NO: 74

SEQ ID NO: 84 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 85-86 - skipped (2 entries)

SEQ ID NO: 87 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 88 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 89 - protein, 795 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRDSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 90 - protein, 644 aa, synthetic construct
KAYDQQLKAGDNPYLRYNHADAEFQERLQEDTSFGGNLGRAVFQAKKRVLEPLGLVETPAKTAPGKKRPVDSPDSTSGIGKKGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNXRNSLVNPGVAMATHKDDEERFFPSSGVLIFGKTGAANKTTLENVLMTNEEEIRPTNPVATEEYGIVSSNLQAASTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEP

SEQ ID NO: 91 - protein, 735 aa, synthetic construct; identical to SEQ ID NO: 75

SEQ ID NO: 92 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 93 - protein, 735 aa, synthetic construct; identical to SEQ ID NO: 74

SEQ ID NO: 94 - protein, 735 aa, synthetic construct; identical to SEQ ID NO: 76

SEQ ID NO: 95 - protein, 734 aa, synthetic construct; identical to SEQ ID NO: 77

SEQ ID NO: 96 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPASPSGLGTNTMATGSGAPMADNNEGADGVGNSSGNWHCDSTWMGDRVVTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDLEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 97 - protein, 738 aa, synthetic construct; identical to SEQ ID NO: 84

SEQ ID NO: 98 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 99-100 - skipped (2 entries)

SEQ ID NO: 101 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagaccgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaagcagcggcaagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 102 - DNA, 4093 bp, synthetic construct
  source 1..4093: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagaccgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaagcagcggcaagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 103 - DNA, 1806 bp, synthetic construct
  source 1..1806: mol_type = other DNA
ggaggctctgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaagcagcggcaagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 104 - DNA, 2082 bp, synthetic construct
  source 1..2082: mol_type = other DNA
atggacctgggcaagaagctgcttgaggccgctagagccggccagaacgacgaggtgcggatcctgatggccaacggcgccgacgtgaacgccatcgacatggccggaagaacacctctgcacctcgccgcttggtccggccacctggaaatcgtggaagtgctgctgaagtacgacgccgatgtgaacgccaccgaccacttcggcctgacccctctgcacctggccgccagcgacggccacctggacatcgccgaggtgctgcagaaggccgccggaggctctgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaagcagcggcaagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 105 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagaccgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 106 - DNA, 4093 bp, synthetic construct
  source 1..4093: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagaccgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 107 - DNA, 1806 bp, synthetic construct
  source 1..1806: mol_type = other DNA
ggaggctctgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 108 - DNA, 2082 bp, synthetic construct
  source 1..2082: mol_type = other DNA
atggacctgggcaagaagctgcttgaggccgctagagccggccagaacgacgaggtgcggatcctgatggccaacggcgccgacgtgaacgccatcgacatggccggaagaacacctctgcacctcgccgcttggtccggccacctggaaatcgtggaagtgctgctgaagtacgacgccgatgtgaacgccaccgaccacttcggcctgacccctctgcacctggccgccagcgacggccacctggacatcgccgaggtgctgcagaaggccgccggaggctctgctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 109 - DNA, 2208 bp, synthetic construct
  source 1..2208: mol_type = other DNA
gctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 110 - DNA, 4093 bp, synthetic construct
  source 1..4093: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggttaggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 111 - DNA, 2208 bp, synthetic construct; identical to SEQ ID NO: 109
  source 1..2208: mol_type = other DNA

SEQ ID NO: 112 - DNA, 4093 bp, synthetic construct; identical to SEQ ID NO: 110
  source 1..4093: mol_type = other DNA

SEQ ID NO: 113 - protein, 622 aa, synthetic construct
GSPTPTPTTPTPTPTTPTPTPTGSAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - protein, 778 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPTPTPTTPTPTPTTPTPTPTGSGSPTPTPTTPTPTPTTPTPTPTGSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 779 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAGSPTPTPTTPTPTPTTPTPTPTGSGSPTPTPTTPTPTPTTPTPTPTGSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - DNA, 1863 bp, synthetic construct
  source 1..1863: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 117 - protein, 593 aa, synthetic construct
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 118 - protein, 641 aa, synthetic construct
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGGGSGGGGSGGGGSGGGGSGSGSGGGGSGGGGSGGGGSGGGGSGSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 119 - protein, 730 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - DNA, 3662 bp, synthetic construct
  source 1..3662: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagccaccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggctctggcggcggcggcagcggcggaggcggctccggcggaggcggcagcggcggaggaggttctggaagcggctctggcggcggcggcagcggcggaggcggctccggcggaggcggcagcggcggaggaggttctggaagccaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 121 - protein, 736 aa, synthetic construct; identical to SEQ ID NO: 31

SEQ ID NO: 122 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 743 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - protein, 593 aa, synthetic construct
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 126 - protein, 7 aa, synthetic construct
VQVGRTS

SEQ ID NO: 127 - protein, 5 aa, synthetic construct
GGGGS

SEQ ID NO: 128 - protein, 595 aa, synthetic construct
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 129 - protein, 641 aa, synthetic construct; identical to SEQ ID NO: 118

SEQ ID NO: 130 - protein, 641 aa, synthetic construct; identical to SEQ ID NO: 118

SEQ ID NO: 131 - protein, 532 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 132 - protein, 578 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGGGSGGGGSGGGGSGGGGSGSXNGSGGGGSGGGGSGGGGSGGGGSGSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 133 - protein, 577 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGGGSGGGGSGGGGSGGGGSGSXGSGGGGSGGGGSGGGGSGGGGSGSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 134 - protein, 736 aa, synthetic construct; identical to SEQ ID NO: 67

SEQ ID NO: 135 - protein, 10 aa, synthetic construct
GGGGSGGGGS

SEQ ID NO: 136 - protein, 15 aa, synthetic construct
GGGGSGGGGSGGGGS

SEQ ID NO: 137 - protein, 20 aa, synthetic construct
GGGGSGGGGSGGGGSGGGGS

SEQ ID NO: 138 - protein, 24 aa, synthetic construct
GSGGGGSGGGGSGGGGSGGGGSGS

SEQ ID NO: 139 - protein, 20 aa, synthetic construct
PTPTPTTPTPTPTTPTPTPT

SEQ ID NO: 140 - protein, 24 aa, synthetic construct
GSPTPTPTTPTPTPTTPTPTPTGS

SEQ ID NO: 141 - protein, 528 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 142 - protein, 578 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNGSGGGGSGGGGSGGGGSGGGGSGSXNGSGGGGSGGGGSGGGGSGGGGSGSQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2026/0308615 A1
(43) Pub. Date: Oct. 8, 2026

(54) TARGETING AAV CAPSIDS, METHODS OF MANUFACTURING AND USING SAME

(71) Applicant: REGENXBIO INC., Rockville, MD (US)

(72) Inventors: Elad Firnberg, Baltimore, MD (US); Andrew Mercer, Pooleville, MD (US)

(21) Appl. No.: 19/473,971

(22) PCT Filed: Apr. 14, 2024
(86) PCT No.: PCT/US2024/024545

§ 371 Date: Oct. 9, 2025

Related U.S. Application Data
(60) Provisional application No. 63/459,238, filed on Apr. 13, 2023.
(60) Provisional application No. 63/502,633, filed on May 16, 2023.
(60) Provisional application No. 63/592,563, filed on Oct. 23, 2023.

(51) Int. Cl. C12N 15/86; A61K 48/00; C07K 14/005

(52) U.S. Cl. CPC C12N 15/86; A61K 48/005; C07K 14/005; C12N 2750/14122; C12N 2750/14143; C12N 2750/14145; C12N 2750/14151; C12N 2810/40

(57) ABSTRACT

The present invention relates to recombinant adeno-associated viruses (rAAVs) having either wild-type capsid proteins or capsid proteins engineered to be atropic or have limited tropism which may be further engineered by insertion of a DARPin targeting element that confers and/or enhances desired properties, particularly increased transduction in CNS or muscle cells or other target tissue relative to a rAAV having a reference capsid.

### REFERENCE TO SEQUENCE LISTING

The contents of the electronic sequence listing (38013_0034P1_41324V2.xml; Size: 214,525 bytes; and Date of Creation: Apr. 13, 2024) is herein incorporated by reference in its entirety.

### 1. FIELD OF THE INVENTION

The present invention relates to recombinant adeno-associated viruses (rAAVs) having capsid proteins with one or more amino acid substitutions and/or peptide insertions that confer and/or enhance desired properties, including tissue tropisms. In particular, engineered capsids may be engineered to display a targeting domain, such as a peptide, antibody or designed ankyrin repeat proteins (DARPin) comprising a binding domain, to enhance transduction of one or more tissue types as compared to the engineered capsid that does not comprise the targeting domain and/or as compared to AAV9 or AAV with another parental capsid and, in embodiments, preferentially transduces the target tissue and does not transduce other tissue types which are not targeted. The engineered capsids may comprise a VP1, VP2, and/or VP3 protein comprising a DARPin in addition to either wild-type and/or reduced tissue tropism VP1, VP2 and/or VP3 proteins. rAAVs having the capsid proteins disclosed herein are useful for delivering a transgene encoding a therapeutic protein or nucleic acid for treatment of disease associated with the tissue in which transduction of the engineered rAAV is enhanced.

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and over twenty gene therapy products are currently in clinical development. It may be useful to design AAV capsids which have enhanced transduction of specific tissue types such as, but not limited to, CNS, muscle and/or heart tissue while also exhibiting reduced transduction of tissue types such as, liver and/or dorsal root ganglion cells and/or kidney may also be desirable to reduce toxicity.

While some strategies for re-targeting of AAV vectors by insertion of targeting ligands into a capsid sequence have failed, due to the reducing conditions in the cell nucleus that effect the assembly of the AAV particles, designed ankyrin repeat proteins (DARPins) have emerged as promising targeting ligands to re-direct AAV. The molecular engineering feat to insert a large targeting sequence, such as a DARPin, into an AAV capsid coding sequence is unpredictable due to the constraints of capsid assembly dynamics such as VP ratios, protein folding, thermostability, and intramolecular interactions (steric effects, Vander waals forces, etc.), to name a few.

### 3. SUMMARY OF THE INVENTION

Provided are recombinant adeno-associated viruses (rAAVs) having capsid proteins engineered to efficiently produce rAAV vectors that contain relatively large protein insertions, such as DARPins. DARPins are engineered, in some embodiments, to target cell membrane receptors of interest and when inserted into the capsid protein have been shown to re-direct the rAAV to the cell or tissue of interest. Multiple parameters of DARPin-AAV fusion constructs and production of the rAAV vectors were engineered including the VP insertion site, linker length, linker composition, VR mutations, and transfection conditions. DARPins of different repeat numbers, binding affinities, and receptor targets were also tested such that target receptors and their method of action on or within certain tissues can be harnessed to ferry rAAV particles.

Using the engineered AAV-DARPin construct, vector production titer and VP ratios could be maintained to near those of the unmodified (parent) vector. It was found that DARPin insertions in VR-IV of VP2 (such as Capsid L, see FIG. 2F) could mediate 90-fold increased transduction of HEK293 cells over-expressing target receptors (that bind the DARPin) compared to the unmodified vector. Insertions in VR-VIII (see FIG. 2E) or at the N-terminus of VP2 (see FIG. 2C) increased transduction but to a lesser extent while the linker composition (glycine-serine linker vs proline-threonine linker) or length had minimal impact on rAAV transduction activity.

Provided are also recombinant adeno-associated viruses (rAAVs) having capsid proteins with polypeptide inserts of about 60 to more than about 200 amino acids, including of polypeptides that target certain cell surface proteins, and that are further engineered to have one or more amino acid substitutions that reduce or obviate or increase or modify targeting, transduction and/or integration of the rAAV genome in mammalian, including human, tissues, including all tissues of the subject or a subset of tissues, such as, but not limited to, one or more of liver, heart, skeletal muscle (including biceps, transverse abdominal muscle, gastrocnemius muscle, or quadriceps), cardiac muscle, brain, kidney, lung, pancreas, meniscus, and/or peripheral nervous system relative to a reference capsid, for example, the parent capsid, including an AAV8 or AAV9 or AAVhu32 capsid. Such “atropic” or limited tropic capsids may also be termed “detargeted” for one or more tissue types. The atropic capsids may then be further engineered to incorporate or insert the targeting moiety as discussed hereinabove, such as a DARPin, peptide, antibody, or other molecule, which has a targeting and/or binding domain that enhances transduction of the rAAV in one or more specific tissue types, such as, for example but not limited to, CNS, muscle, or heart, relative to the parent atropic (or limited tropic) capsid (having the one or more amino acid substitutions) and/or the parent capsid, including AAV8 or AAV9 or AAVhu32. Biodistribution studies in mice and non-human primates permit assessment of relative transduction and transgene transcription and expression in various tissue types of capsids, including engineered capsids (see, the Examples, infra).

In particular, provided are AAV9 capsid proteins or AAV8 capsid proteins (SEQ ID NO:69 or 63, respectively, and as numbered in FIG. 8) or AAVhu32 or other AAV type capsid having one or more amino acid substitutions (including, 2, 3 or 4 amino acid substitutions) that reduce or obviate relative to the parent AAV (e.g., AAV8 or AAV9 or AAVhu32) targeting and/or transduction of the engineered rAAV of one or more tissue types (or all tissue types) of a subject (including a mammalian, rodent, primate or human subject), including liver, heart, lung, kidney, pancreas, skeletal muscle (including biceps, transabdominal, gastrocnemius, quadriceps) or cardiac muscle, meniscus, brain and/or peripheral nervous system tissue. Such amino acid modifications include G266A, N272A, W503A or W503R of AAV9, and corresponding substitutions in other AAV type capsids (for example according to the alignment in FIG. 8), The capsids having these amino acid substitutions may further have substitutions of the NNN (asparagines) at 496 to 498 with AAA (alanines) of the AAV9 capsid or at positions 498 to 500 of the AAV8 capsid, or corresponding substitutions in other AAV type capsids.

Engineered capsids include VP1 modifications including a peptide insert of VQVGRTS (SEQ ID NO: 126) inserted between S454 and G454 (herein NVG07) or inserted in another AAV capsid at a corresponding position (see, e.g., FIG. 8).

Engineered capsids include VP1 modifications such as AAV9.G266A.496-NNN/AAA-498 (SEQ ID NO:50). AAV9.N272A.497-NNN/AAA-498 (SEQ ID NO:49), AAV9.496-NNN/AAA-498.W503R (SEQ ID NO:32), AAV9.496-NNN/AAA-498.W503A (SEQ ID NO:51), AAV9.496-NNN/AAA-498 (SEQ ID NO: 121), AAV9.496-NNN/AAA-498.NVG07 (SEQ ID NO: 122), AAVhu.32.496-NNN/AAA-498 (SEQ ID NO: 123), and AAVhu.32.496-NNN/AAA-498.NVG07 (SEQ ID NO: 124).

Provided are recombinant AAV vectors that incorporate VP1 engineered capsid proteins, VP2 engineered capsid protein, and/or VP3 engineered capsid proteins comprising a DARPin insertion. In embodiments, the engineered capsids may comprise a VP1, VP2, and/or VP3 protein comprising a DARPin in addition to either wild-type and/or reduced tissue tropism VP1. VP2 and/or VP3 proteins. In embodiments, during manufacture, the VP1. VP2, and/or VP3 comprising the DARPin insertion are provided at an appropriate ratio with wild-type and/or reduced tissue tropism VP1, VP2 and/or VP3 proteins, for example, by transfecting cells with ratios of polynucleotides that encode the wild-type and/or reduced tissue tropism VP1, VP2, and/or VP3 proteins and polynucleotides that encode the VP1, VP2, or VP3 having the DARPin insertion to engineer capsids that have both enhanced manufacturability and tissue and/or cell tropism.

These atropic (or limited tropic) or liver detargeting capsids may be further engineered to include a heterologous molecule, including a polypeptide, such as a peptide, antibody or antigen binding domain thereof (including single domain antibodies) or other binding or targeting domain, such as a DARPin, such that the polypeptide or other molecule is displayed on the surface of the rAAV when the engineered capsid protein is incorporated into the capsid, and confers tissue tropism onto the AAV particles having the engineered capsid relative to the atropic or reduced tropic capsid (or even the parental capsid engineered to make the atropic capsid). The peptide, antibody, antigen binding domain thereof, DARPin or other binding or targeting domain may be inserted at an appropriate position in the VP1,VP2, and/or VP3 capsid protein, including at or near the VP2 initiation codon, or within the VR-1 region, VR-IV region, or VR-VIII region.

For example, engineered capsids comprise VP1 AAV9.G266A.496-NNN/AAA-498 (SEQ ID NO:50) and VP2 AAV9.linker.darpin.linker (SEQ ID NO: 125), or VP1 AAV9.N272A.497-NNN/AAA-498 (SEQ ID NO:49) and VP2 AAV9.linker.darpin.linker (SEQ ID NO: 125), or AAV9.496-NNN/AAA-498.W503R (SEQ ID NO:32) and VP2 AAV9.linker.darpin.linker (SEQ ID NO: 125), or AAV9.496-NNN/AAA-498.W503A (SEQ ID NO:51) and VP2 AAV9.linker.darpin.linker (SEQ ID NO: 125), or VP1 AAV9.496-NNN/AAA-498 (SEQ ID NO: 121) and VP2 AAV9.linker.darpin.linker (SEQ ID NO: 125), VP1 AAV9.496-NNN/AAA-498.NVG07 (SEQ ID NO: 122) and VP2 AAV9.linker.darpin.linker (SEQ ID NO: 125), or VP1 AAVhu.32.496-NNN/AAA-498 (SEQ ID NO: 123) and VP2 AAV9.linker.darpin.linker (SEQ ID NO: 125), or VP1 AAVhu.32.496-NNN/AAA-498.NVG07 (SEQ ID NO: 124) and VP2 AAV9.linker.darpin.linker (SEQ ID NO: 125). In embodiments, the engineered capsids may comprise a VP1, VP2, and/or VP3 protein comprising a DARPin in addition to either wild-type and/or reduced tissue tropism VP1, VP2 and/or VP3 proteins. For example, in certain VP1 constructs, the VP1 capsid protein sequence has a mutated VP2 start codon (nonfunctional) and both VP1 and VP2 constructs utilized to produce the capsid encode VP3 proteins. In another example, a secondary VP3 construct comprises a DARPin insert in VR4 and a trans construct provides wild type or reduced tropism VP1, VP2, and VP3 capsid proteins (i.e., VP1, VP2 and VP3 capsid proteins which do not have the DARPin insert).

In another example, the peptide, antibody, antigen binding domain thereof. DARPin or other binding or targeting domain may be inserted at one of position 138, 262-273, 452-461, or 585-593 for AAV9 according to the numbering for the AAV9 VP1 protein (FIG. 8) or corresponding position for a different AAV capsid. Also provided are capsids, particularly AAV9 capsids but including other capsid types, that have one of G266A, N272A, W503A or W503R amino acid substitution and 496-NNN/AAA-498 amino acid substitutions (or corresponding substitutions in a different AAV serotype) and also have a peptide TLAAPFK (SEQ ID NO:1) inserted between Q588 and A589 (herein PHP.hDYN) or alternatively at an appropriate position, including between S268 and S269 or between S454 and G455 for AAV9 according to the numbering for the AAV9 VP1 protein (FIG. 8) or inserted in another AAV capsid at a corresponding position (see, e.g., FIG. 8), or at any other position that displays the peptide on the capsid surface to promote tissue specific binding and transduction, for example, including at or near the VP2 initiation codon, or within the VR-1 region, VR-IV region, or VR-VIII region. Or, alternatively, the capsid is an AAV9 PHP.eB capsid (which has the modifications A587D and Q588G) which comprises one of the amino acid substitutions of G266A, W503A or W503R and the amino acid substitutions 496-NNN/AAA-498 and further insertion of the peptide TLAVPFK (SEQ ID NO:20) between G588 and A589 and the peptide TILSRSTQTG (SEQ ID NO:15) between position 138 and 139 for AAV9 according to the numbering for the AAV9 VP1 protein (FIG. 8), or the corresponding position in a different capsid, or at any other position that displays the peptide on the capsid surface to promote tissue specific binding and transduction, for example, including at or near the VP2 initiation codon, or within the VR-1 region, VR-IV region, or VR-VIII region. Provided are recombinant AAV capsids comprising an amino acid substitution of G266A, N272A, W503A or W503R and the amino acid substitutions 496-NNN/AAA-498 of AAV9 according to the numbering for the AAV9 VP1 protein (FIG. 8) (or corresponding amino acid substitution in another capsid) and insertion of the peptide RTIGPSV (SEQ ID NO:12), including inserted between positions 138 and 139, positions S454 and G455, or positions Q588 and A589 of AAV9 according to the numbering for the AAV9 VP1 protein (FIG. 8), or corresponding position of another AAV capsid, or at any other position that displays the peptide on the capsid surface to promote tissue specific binding and transduction, for example, including at or near the VP2 initiation codon, or within the VR-1 region, VR-IV region, or VR-VIII region. Also provided are additional capsids comprising an amino acid substitution of G266A, N272A, W503A or W503R and the amino acid substitutions 496-NNN/AAA-498 of AAV9 according to the numbering for the AAV9 VP1 protein (FIG. 8) (or corresponding amino acid substitution in another capsid) and which have a Kidney1 peptide LPVAS (SEQ ID NO:6) inserted into the capsid, for example between S454 and G455 of AAV9, or alternatively between S268 and S269 or between Q588 and A589 of AAV9 according to the numbering for the AAV9 VP1 protein (FIG. 8), or the corresponding position of a different capsid, or at any other position that displays the peptide on the capsid surface to promote tissue specific binding and transduction, for example, including at or near the VP2 initiation codon, or within the VR-1 region, VR-IV region, or VR-VIII region.

Accordingly, provided herein are rAAVs with enhanced or increased biodistribution, including transduction, genome integration, transgene transcription and expression, in CNS tissues (including frontal cortex, hippocampus, cerebellum, midbrain) relative to a reference capsid (for example the parental capsid that has the amino acid substitution reducing tissue targeting or transduction, or AAV8 or AAV9 or AAVhu32 or variants thereof), with reduced distribution, including transduction, genome integration, transgene transcription and expression in one or more of the heart, liver, lung, kidney, pancreas, meniscus, muscle, and/or dorsal root ganglion cells (cervical, thoracic, and/or lumbar) compared to the biodistribution of a reference capsid, such as the parental capsid or AAV8 or AAV9 or AAVhu32 or variants thereof, that do not have the amino acid substitutions and insertion of the targeting domain. Such rAAVs may be useful to deliver therapeutic proteins or nucleic acids for the treatment of CNS disease or other disease associated with tissues for which the engineered capsid AAV has increased transduction, for example transgenes provided in Table 1A or 1B.

In addition, provided herein are rAAVs with enhanced or increased biodistribution, including transduction, genome integration, transgene transcription and expression, in skeletal muscle and/or cardiac muscle tissues relative to a reference capsid (for example the unengineered, parental capsid or AAV8 or AAV9 or AAVhu32), with reduced distribution, including transduction, genome integration, transgene transcription and expression in the liver, lung, kidney, pancreas, meniscus, brain, and/or dorsal root ganglion cells (cervical, thoracic, and/or lumbar) compared to the biodistribution in skeletal and/or cardiac muscle tissue and/or relative to an AAV with a reference capsid, such as the parental capsid or AAV8 or AAV9 or AAVhu32. Such rAAVs may be useful to deliver therapeutic proteins or nucleic acids for the treatment of muscle disease, including transgenes provided in Table 1A and 1B.

In other embodiments, the targeting domain increases targeting of the rAAV to other tissues such as lung, kidney, pancreas, peripheral nervous system.

In certain embodiments, transduction is measured by detection of transgene, such as GFP fluorescence.

The capsid protein to be engineered may be an AAV9 capsid protein but may also be any AAV capsid protein, such as AAV serotype 1 (SEQ ID NO:59); AAV serotype 2 (SEQ ID NO:60); AAV serotype 3 (SEQ ID NO:61), AAV serotype 3-3 (SEQ ID NO:78); AAV serotype 3B (SEQ ID NO:87): AAV serotype 4 (SEQ ID NO:62): AAV serotype 4-4 (SEQ ID NO:79); AAV serotype 5 (SEQ ID NO:63); AAV serotype 6 (SEQ ID NO:64); AAV serotype 7 (SEQ ID NO:65); AAV serotype 8 (SEQ ID NO:66); AAV serotype 9 (SEQ ID NO:67); AAV serotype 9e (SEQ ID NO:68); AAV serotype rh10 (SEQ ID NO:69); AAV serotype rh20 (SEQ ID NO:70); and AAV serotype hu.37 (SEQ ID NO:71). AAV serotype rh39 (SEQ ID NO:73), and AAV serotype rh74 (SEQ ID NO:72 or SEQ ID NO:80), AAV serotype rh.34 (SEQ ID NO:82), AAV serotype hu.60, AAV serotype rh.21 (SEQ ID NO:83), AAV serotype rh.15, AAV serotype rh.24, AAV serotype hu.5, AAV serotype hu.10, AAV serotype rh64R1 (SEQ ID NO:48), AAV serotype rh46 (SEQ ID NO:84), and AAV serotype rh73 (SEQ ID NO:88) (see FIG. 8 for alignment of certain sequences) and Table 7 for capsid (VP1/VP2/VP3 or VP1/VP3 or VP2) amino acid sequences and Table 8 for capsid nucleotide sequences.

In certain embodiments, provided are rAAVs incorporating the engineered capsids described herein, including rAAVs with genomes comprising a transgene of therapeutic interest, including a transgene encoding a therapeutic protein or nucleic acid for treatment of a muscle, heart or CNS disease or other disease associated with tissue for which the engineered AAV has increased tropism (see, for example, the transgenes in Tables 1A and 1B). Packaging cells for producing the rAAVs described herein are provided which comprise nucleic acids encoding an engineered capsid described herein under the control of appropriate regulatory elements. Packaging cells for producing the rAAVs described herein are provided which comprise a first nucleic acid sequence encoding a capsid protein comprising an inactivated VP2 initiation site (thus only expressing VP1 and VP3) and comprise a second nucleic acid sequence encoding a VP2 capsid protein. In embodiments, the VP2 capsid protein has a linker-darpin-linker insert as described herein. Packaging cells for producing the rAAVs described herein are provided which comprise a first nucleic acid sequence encoding a capsid protein comprising an inactivated VP3 initiation site (thus only expressing VP1 and VP2) and comprise a second nucleic acid sequence encoding a VP3 capsid protein. In embodiments, the VP3 capsid protein has a linker-darpin-linker insert as described herein.

Method of treatment by delivery of, and pharmaceutical compositions comprising, the engineered rAAVs described herein are also provided. Also provided are methods of manufacturing the rAAVs with the engineered capsids described herein. Provided are nucleic acids encoding the engineered capsid proteins, plasmid vectors, such as “RepCap” constructs in which the Cap gene encodes an engineered capsid described herein as well as host cells, such as bacterial host cells, for replication and production of these plasmid vectors.

Provided are methods of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP3-DARPin protein, which comprises a VP3 protein having a DARPin inserted within VR-IV or VR-VIII of the VP3 protein: (b) a polynucleotide encoding a functional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

In embodiments, the DARPin is inserted into a VP1 protein. In embodiments, the DARPin is inserted into a VP2 protein. In embodiments, the DARPin is inserted into a VP3 protein. In embodiments, the additional VP1, VP2 and/or VP3 proteins are incorporated into the engineered capsid and the VP1, VP2 and VP3 proteins may further incorporate amino acid modifications (including substitutions) that reduce tropism as described herein. In embodiments, the additional VP1, VP2 and/or VP3 proteins are wild-type VP1, VP2, and/or VP3 proteins. In embodiments, the additional VP1, VP2 and/or VP3 proteins comprise “detargeted” VP1, VP2, and/or VP3 proteins.

The invention is illustrated by way of examples infra describing the construction of rAAV9 or rAAVhu32, including capsids engineered with amino acid substitutions and/or insertions and assaying of tissue distribution when administered to mice or non-human primates.

### 3.1. Embodiments

Embodiment 1. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP3-DARPin protein, which comprises a VP3 protein having a DARPin inserted within VR-IV or VR-VIII of the VP3 protein; (b) a polynucleotide encoding afunctional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

Embodiment 2. The method of embodiment 1, wherein (a) comprises: (i) a polynucleotide encoding wild-type or parental VP1, VP2 and VP3 proteins and (ii) a polynucleotide encoding VP3-DARPin protein.

Embodiment 3. The method of embodiment 1, wherein (a) comprises: (i) a polynucleotide encoding wild-type or parental VP1 and VP2 proteins, wherein the start codon for VP3 is mutated, and (ii) a polynucleotide encoding VP3-DARPin protein

Embodiment 4. The method of embodiment 2 or embodiment 3, wherein (i) the polynucleotide encoding wild-type or parental VP1, VP2 and VP3 proteins or the polynucleotide encoding wild-type or parental VP1 and VP2 proteins and (ii) the polynucleotide encoding functional rep gene are operably linked.

Embodiment 5. The method of any one of embodiments 2 to 4, wherein prior to the step of culturing the cell comprising one or more polynucleotides, (i) the polynucleotide encoding wild-type or parental VP1, VP2 and VP3 proteins or the polynucleotide encoding wild-type or parental VP1 and VP2 proteins and (ii) the polynucleotide encoding VP3-DARPin protein were introduced into the cell on separate plasmids.

Embodiment 6. The method of embodiment 5, wherein prior to the step of culturing a cell comprising one or more polynucleotides, (i) the polynucleotide encoding wild-type or parental VP1, VP2 and VP3 proteins or the polynucleotide encoding wild-ty pe or parental VP1 and VP2 proteins and (ii) the polynucleotide encoding VP3-DARPin protein were introduced into the cell at a ratio of about 50:50, about 70:30, about 80:20 or about 90:10.

Embodiment 7. The method of any one of embodiments 1 to 6, wherein the DARPin is inserted into the VP3 protein at VR-IV.

Embodiment 8. The method of any one of embodiments 1 to 7, wherein the DARPin is flanked on either one or both ends by a linker.

Embodiment 9. The method of embodiment 8, wherein the linker is a GS linker or a PT-linker.

Embodiment 10. The method of any one of embodiments 1 to 9, wherein the polynucleotide encoding VP3-DARPin protein encodes SEQ ID NO: 132 or SEQ ID NO: 133.

Embodiment 11. An engineered rAAV particle made by the method of any one of embodiments 1 to 10.

Embodiment 12. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP1-DARPin protein, which comprises a VP1 protein having a DARPin inserted within VR-IV or VR-VIII of the VP1 protein: (b) a polynucleotide encoding afunctional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

Embodiment 13. The method of embodiment 12, wherein (a) comprises: (i) a polynucleotide encoding wild-type or parental VP1, VP2 and VP3 proteins and (ii) a polynucleotide encoding VP1-DARPin protein.

Embodiment 14. The method of embodiment 12, wherein (a) comprises: (i) a polynucleotide encoding wild-type or parental VP2 and VP3 proteins, wherein the start codon for VP1 is mutated, and (ii) a polynucleotide encoding VP1-DARPin protein.

Embodiment 15. The method of embodiment 13 or embodiment 14, wherein (i) the polynucleotide encoding wild-type or parental VP1, VP2 and VP3 proteins or the polynucleotide encoding wild-type or parental VP2 and VP3 proteins and (ii) the polynucleotide encoding functional rep gene are operably linked.

Embodiment 16. The method of any one of embodiments 13 to 15, wherein prior to the step of culturing the cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein were introduced into the cell on separate plasmids.

Embodiment 17. The method of embodiment 16, wherein prior to the step of culturing a cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein are introduced into the cell at a ratio of about 50:50, about 70:30, about 80:20 or about 90:10.

Embodiment 18. The method of any one of embodiments 12 to 17, wherein the DARPin is inserted into the VP1 protein at VR-IV.

Embodiment 19. The method of any one of embodiments 12 to 18, wherein the DARPin is flanked on either one or both ends by a linker.

Embodiment 20. The method of embodiment 19, wherein the linker is a GS linker or PT linker.

Embodiment 21. The method of any one of embodiments 12 to 20, wherein the polynucleotide encoding VP1-DARPin protein encodes SEQ ID NO: 114 or SEQ ID NO: 115.

Embodiment 22. An engineered rAAV particle made by the method of any one of embodiments 12 to 21.

Embodiment 23. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP2-DARPin protein, which comprises a VP2 protein having a DARPin inserted within or at the VR-IV or VR-VIII of the VP2 protein; (b) a polynucleotide encoding afunctional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

Embodiment 24. The method of embodiment 23, wherein (a) comprises: (i) a polynucleotide encoding wild-type or parental VP1, VP2 and VP3 proteins and (ii) a polynucleotide encoding VP2-DARPin protein.

Embodiment 25. The method of embodiment 23, wherein (a) comprises: (i) a polynucleotide encoding wild-type or parental VP1 and VP3 proteins and (ii) a polynucleotide encoding VP2-DARPin protein.

Embodiment 26. The method of embodiment 24 or embodiment 25, wherein (i) the polynucleotide encoding wild-type or parental VP1. VP2 and VP3 proteins or the polynucleotide encoding wild-type or parental VP1 and VP3 proteins and (ii) the polynucleotide encoding functional rep gene are operably linked.

Embodiment 27. The method of any one of embodiments 24 to 26, wherein prior to the step of culturing the cell comprising one or more polynucleotides. (i) the polynucleotide encoding wild-type or parental VP1, VP2 and VP3 proteins or the polynucleotide encoding wild-type- or parental VP1 and VP3 proteins and (ii) the polynucleotide encoding VP2-DARPin protein were introduced into the cell on separate plasmids.

Embodiment 28. The method of embodiment 27, wherein prior to the step of culturing a cell comprising one or more polynucleotides, (i) the polynucleotide encoding wild-type or parental VP1, VP2 and VP3 proteins or the polynucleotide encoding wild-type or parental VP1 and VP3 proteins and (ii) the polynucleotide encoding VP2-DARPin protein are introduced into the cell at a ratio of about 50:50, about 70:30, about 80:20 or about 90:10.

Embodiment 29. The method of any one of embodiments 23 to 28, wherein the DARPin is inserted into the VP2 protein at VR4.

Embodiment 30. The method of any one of embodiments 23 to 29, wherein the DARPin is flanked on either one or both ends by a linker.

Embodiment 31. The method of embodiment 30, wherein the linker is a GS linker or a PT linker.

Embodiment 32. The method of any one of embodiments 23 to 31, wherein the polynucleotide encoding VP1-DARPin protein encodes SEQ ID NO: 119.

Embodiment 33. An engineered rAAV particle made by the method of any one of embodiments 23 to 32.

Embodiment 34. A recombinant AAV capsid protein comprising an insertion of a DARPin which targets a cell surface molecule and wherein the DARPin is flanked on at least one end by a linker, wherein a recombinant AAV particle incorporating the recombinant AAV capsid protein has cell transduction activity.

Embodiment 35. The recombinant AAV capsid protein of embodiment 34, wherein the AAV capsid protein is an AAV9 or AAVhu32 capsid protein and comprises no other substitutions or insertions.

Embodiment 36. The recombinant AAV capsid protein of embodiment 34, which further comprises one or more amino acid substitutions and/or insertions relative to the wild type or unengineered capsid protein which when incorporated into an rAAV capsid exhibits reduced transduction or exhibits increased transduction of at least one tissue type relative to an rAAV capsid incorporating the wild type or unengineered capsid protein.

Embodiment 37. The recombinant AAV capsid protein of embodiment 36, in which the rAAV capsid protein has (1) a G266A substitution, a N272A substitution, a W503A substitution or a VQVGRTS insertion between 454 and 455 or (2) 496-NNN/AAA-498 substitutions, or (3) a combination thereof, for an AAV9 capsid protein, or corresponding substitutions in a capsid protein of another AAV type capsid.

Embodiment 38. The recombinant AAV capsid protein of embodiment 37 which comprises N272A and 496-NNN/AAA-498 substitutions.

Embodiment 39. The recombinant AAV capsid protein of any one of embodiments 34 to 38 wherein the insertion of the DARPin is near the VP2 initiation codon or within the VR-1 region. VR-IV region, or VR-VIII region of the VP2 protein.

Embodiment 40. The recombinant AAV capsid protein of any one of embodiments 34 to 39, wherein the insertion is at one of positions 138, 262-273, 452-461, or 585-593, or replaces one or more of amino acids 452-461 or 585-593, for AAV9 as numbered for the VP1 amino acid sequence or corresponding position for a different AAV capsid.

Embodiment 41. The recombinant AAV capsid protein of any one of embodiments 34 to 40, wherein the insertion is between Q588 and A589, S268 and S269, before or after 1451, N452, G453, S454, G455, Q456, N457, Q458, Q459, T460, or L461 of AAV9 as numbered for the VP1 amino acid sequence or corresponding position of a different AAV capsid protein.

Embodiment 42. The recombinant AAV capsid protein of any one of embodiments 34 to 41 wherein the DARPin replaces one or more of amino acids 452-461 or 585-593 of AAV9 as numbered for the VP1 amino acid sequence or corresponding amino acids of a different AAV capsid protein.

Embodiment 43. The recombinant AAV capsid protein of any one of embodiments 34 to 42 wherein the linker comprises a (G)nS linker, where n=2-10.

Embodiment 44. The recombinant AAV capsid protein of any one of embodiments 34 to 43, wherein the linker comprises GGS, GGGGS (SEQ ID NO: 127), 4GSx2 (SEQ ID NO: 135), 4GSx3 (SEQ ID NO: 136), 4GSx4 (SEQ ID NO: 137), GS-4GSx4-GS (SEQ ID NO: 138), GS-PT linker-GS (SEQ ID NO: 139) or GS-PT linker-GS (SEQ ID NO: 140) and is at both the N-terminus and C-terminus of the DARPin insert.

Embodiment 45. The recombinant AAV capsid protein of any one of embodiments 34 to 44 where the linker comprises a PT linker.

Embodiment 46. The recombinant AAV capsid protein of any one of embodiments 34 to 45 wherein the DARPin targets a CNS cell surface protein.

Embodiment 47. The recombinant AAV capsid protein of any one of embodiments 34 to 46, which when incorporated into a rAAV particle, the rAAV particle has increased targeting, binding or transduction into CNS cells, relative to a rAAV particle incorporating the corresponding capsid protein without the DARPin insertion.

Embodiment 48. The recombinant AAV capsid protein of any one of embodiments 34 to 47 wherein the DARPin targets a human receptor or GluA4.

Embodiment 49. The recombinant AAV capsid protein of embodiment 34 which has an amino acid sequence of SEQ ID NO: 113, 114 or 115.

Embodiment 50. The recombinant AAV capsid protein of embodiment 34 or claim 49 which is encoded by the nucleic acid sequences of SEQ ID NOs: 101/103 or 105/107 or 120.

Embodiment 51. The recombinant AAV capsid protein of any one of embodiments 34 to 50 which is a VP1 or VP2 or VP3 capsid protein.

Embodiment 52. A nucleic acid comprising a nucleotide sequence encoding the rAAV capsid protein of any one of embodiments 34 to 51, or encoding an amino acid sequence sharing at least 80% identity therewith and retaining biological activity of the rAAV capsid protein, optionally wherein the nucleotide sequence encoding the rAAV capsid protein is operably linked to a promoter and a polyadenylation sequence.

Embodiment 53. The nucleic acid of embodiment 52 encoding the rAAV capsid protein of any one of claims 28 to 45.

Embodiment 54. A plasmid vector comprising the nucleic acid of embodiment 52 or claim 53, which is replicable in a bacterial cell.

Embodiment 55. A bacterial host cell comprising the plasmid vector of embodiment 54.

Embodiment 56. A packaging cell which expresses the nucleic acid of embodiment 52 or claim 53 to produce AAV particles comprising the capsid protein encoded by said nucleotide sequence.

Embodiment 57. The packaging cell of embodiment 56, wherein the nucleic acid encodes a recombinant VP2 capsid protein and the packaging cell further comprises a nucleic acid which expresses AAV VP1 and VP3 capsid proteins, optionally having a mutated VP2 start codon.

Embodiment 58. The packaging cell of embodiment 56 wherein the nucleic acid encodes a recombinant VP1 capsid protein and the packaging cell further comprises a nucleic acid which expresses AAV VP2 and VP3 capsid proteins, optionally having a mutated VP1 start codon.

Embodiment 59. An rAAV particle comprising the rAAV capsid protein of any one of embodiments 34 to 51.

Embodiment 60. The rAAV particle of embodiment 59, wherein the insertion of the DARPin is (1) in the recombinant VP1 capsid protein but not the VP2 or VP3 capsid protein; (2) in the recombinant VP2 capsid but not in the VP1 or VP3 capsid protein or (3) in the recombinant VP3 capsid but not in the VP1 or VP2 capsid protein.

Embodiment 61. The rAAV particle of embodiment 59 or embodiment 60 further comprising a nucleic acid comprising a transgene encoding a therapeutic protein or a therapeutic nucleic acid operably linked to a regulatory sequence for expression of the therapeutic protein or the therapeutic nucleic acid in the target cells or tissue, wherein the transgene and regulatory sequence are flanked by AAV ITR sequences.

Embodiment 62. The rAAV particle of embodiment 61, wherein the regulatory sequence promotes expression of the therapeutic protein or therapeutic nucleic acid in muscle or CNS cells.

Embodiment 63. A pharmaceutical composition comprising the rAAV particle of any one of embodiments 59 to 62 and a pharmaceutically acceptable carrier.

Embodiment 64. A method of delivering a transgene to a cell, said method comprising contacting said cell with the rAAV particle of any of embodiments 59 to 62 wherein said transgene is delivered to said cell.

Embodiment 65. The method of embodiment 64 in which the cell is a CNS cell, cardiac muscle cell or skeletal muscle cell.

Embodiment 66. A method of delivering a transgene to a target tissue of a subject having a disease associated with the target tissue and treatable by expression of said transgene in said tissue and in need treatment, said method comprising administering to said subject the rAAV particle of any one of embodiments 59 to 62, wherein the transgene is delivered to and expressed in said target tissue.

Embodiment 67. The method of embodiment 66 wherein the transgene is a muscle disease or heart disease therapeutic and said target tissue is cardiac muscle or skeletal muscle.

Embodiment 68. The method of embodiment 66 or embodiment 67, wherein the rAAV is administered systemically, including intravenously or intramuscularly.

Embodiment 69. The method of any one of embodiment 66 to 68, wherein the transgene is a CNS disease therapeutic and said target tissue is CNS.

Embodiment 70. The method of embodiment 69 wherein the rAAV is administered intrathecally, intracerebroventricularly or intravenously.

Embodiment 71. A pharmaceutical composition for use in delivering a transgene to a cell, said pharmaceutical composition comprising the rAAV particle of any of embodiments 59 to 62, wherein said transgene is delivered to said cell.

Embodiment 72. A pharmaceutical composition for use in delivering a transgene encoding a therapeutic protein or therapeutic nucleic acid to a target tissue of a subject having a disease associated with the target tissue and in need treatment, said pharmaceutical composition comprising the rAAV particle of any of embodiments 59 to 62, wherein the transgene is delivered to said target tissue.

Embodiment 73. A host cell comprising: (a) an artificial genome comprising an expression cassette flanked by AAV inverted terminal repeats (ITRs), wherein the expression cassette comprises a transgene encoding a therapeutic protein operably linked to a regulatory element that promotes transgene expression in target cells; (b) a trans expression cassette lacking AAV ITRs, wherein the trans expression cassette encodes an AAV rep protein and one or more of wild-type or parental VP1, VP2 and VP3 operably linked to expression control elements that drive expression of the AAV rep protein and AAV capsid protein in the host cell in culture and supply the rep and capsid proteins in trans: (c) a secondary expression cassette lacking AAV ITRs, wherein the secondary expression cassette encodes the recombinant AAV capsid protein of any of embodiments 34 to 51; and (d) sufficient adenovirus helper functions to permit replication and packaging of the artificial genome by the AAV capsid proteins.

Embodiment 74. A method of producing recombinant AAVs comprising: (a) culturing a host cell containing: (i) an artificial genome comprising an expression cassette flanked by AAV inverted terminal repeats (ITRs), wherein the expression cassette comprises a transgene encoding a therapeutic protein operably linked to a regulatory element that promotes transgene expression in target cells; (ii) a trans expression cassette lacking AAV ITRs, wherein the trans expression cassette encodes an AAV rep protein and one or more of wild-type or parental VP1, VP2 and VP3 operably linked to expression control elements that drive expression of the AAV rep protein and AAV capsid protein in the host cell in culture and supply the rep and cap proteins in trans; (iii) a secondary expression cassette lacking AAV ITRs, wherein the secondary expression cassette encodes the recombinant AAV capsid protein of any one of embodiments 34 to 51; and (iv) sufficient adenovirus helper functions to permit replication and packaging of the artificial genome by the AAV capsid proteins; and (b) recovering recombinant AAV encapsidating the artificial genome from the cell culture.

### 4. BRIEF DESCRIPTION OF THE FIGURES

FIGS. 1A-C depict various expression cassette arrangements for the capsids needed for rAAV production. rAAV production occurs following transfection of: 1—a rep/cap (trans)-expressing plasmid, needed to form the VPs of the capsid not containing the DARPin insert, 2—a secondary trans plasmid which plasmid also encodes for a DARPin inserted in the capsid protein, 3—a cis plasmid carrying a genome (not depicted), and 4—helper genes (not depicted) to allow for formation of an rAAV particle having a capsid including a surface DARPin encapsidating a genome.

FIGS. 2A-H summarize AAV9 expression cassette arrangements. FIGS. 2A-B depict AAV9 rep/cap (trans) plasmid encoding DARPin insertions located at VP2 N-terminus, VR4 or VR8 of the capsid protein (Capsid J or K), no secondary plasmid is needed. FIGS. 2C-H summarize examples of trans rep/cap and second trans cassettes for making capsids, showing linker and DARPin locations.

FIG. 3 graphs the in vitro transduction activity of AAV9-DARPin vector transduced into HEK293 cells over-expressing rat GluA4.

FIGS. 4A-C represent AAV9-DARPin (e.g. GluA4-targeted DARPin) vectors injected intra-striatally into mouse brain and transgene (GFP, FIG. 4A) expression is visualized using fluorescent imaging techniques: PV+ cells were detected as DAPI positive (stained, FIG. 4B) cells and overlaid (merged, FIG. 4C) with the GFP (transgene) images.

FIGS. 5A-5B illustrate transduction of a VP1.VR4 or VP1.VR8 DARPin-capsid into HEK293 cells overexpressing certain receptors. (A) TdTomato transgene expression was visualized by fluorescence imaging and (B) quantified.

FIGS. 6A-6B depict protein staining (SDS-PAGE/Coomassie blue, FIG. 6A) and Western blot (FIG. 6B) to compare VP1 protein production from various DARPin-capsid formats.

FIGS. 7A-B illustrate transgene expression in a mouse brain section (striatum) following injection of atropic-AAV9 vector (FIG. 7A) or the AAV9-atropic+GluA4-DARPin vector (FIG. 7B).

FIG. 8 depicts alignment of AAVs 1-9e, 3B, rh10, rh20, rh39, rh73, rh74 version 1 and version 2, hu12, hu21, hu26, hu37, hu51 and hu53 capsid sequences with insertion sites for heterologous peptides after the initiation codon of VP2, and within or near variable region 1 (VR-I), variable region 4 (VR-IV), and variable region 8 (VR-VIII), all highlighted in grey; a particular insertion site within variable region eight (VR-VIII) of each capsid protein is shown by the symbol “#” (after amino acid residue 588 according to the amino acid numbering of AAV9).

FIGS. 9A-9D depict VP protein, VP1-DARPin (A) and transgene expression (B) at 48 hrs post-transduction of HEK293-hTargetReceptor (hTR) cells, average titer and copy number of VP1-DARPins detected (C), and VP protein and DARPin expression following one liter (1 L) scale production as measured by SDS-PAGE and Western (D).

FIGS. 10A-C show that DARPins inserted in AAV9 mediated up to 38-fold increased transduction of HEK293-hTargetReceptor cells versus AAV9. TdTomato fluorescence (A and B), RNA copies (B) and GC/cell (B) were measured 48 hours post-transduction of HEK293-hTR and HEK293-AAVR cells at 1E5 MOI with DARPin-AAV vectors. Multiple regression analysis with two independent variables identified a positive correlation between fold-change transduction (FCT) of HEK293-hTR cells, VP1-DARPin copies and DARPin KD (i.e. increased FC-trans, decreased VP-DARPin copies, decreased binding affinity) (C).

FIGS. 11A-B show that the CAG promoter and DARPin insertion in VP2 VR-IV mediated a 90-fold increased transduction versus AAV9 where DARPins targeting a human TR induced greater transduction in the HEK293-hTR cells (FIG. 11A). FIG. 11B shows a chart of the various constructs and their elements.

FIGS. 12A-B show SDS-PAGE and anti-VP1 Western Blots of AAV-DARPin constructs performed for the 50 mL scale AAV-DARPin productions (FIG. 12A), including adequate expression of VP1-DARPin fusion protein. The fold change cell binding of an anti-hTR-DARPin (GTT35) and single-domain anti-hTR antibody (GTT36) from AAV9, and their binding affinities to the target receptor (kD) is shown in FIG. 12B.

FIGS. 13A-C show (A) DARPin-AAV fusions. (B) Engineering the AAV-DARPin fusion site and production methods leads to 90-fold improvement in transduction of cells expressing the DARPin target receptor (TR) based on normalized fluorescent intensity. (C) In vitro transduction of 1st and 2nd generation DARPin AAVs (DARPin1 and DARPin2, respectively).

FIGS. 14A-B. FIG. 14A shows a 3D capsid model having three DARPin binding proteins attached to the capsid surface, and (B) a depiction of initial production titers of combination mutant DARPin-AAV vectors having AAV9 or AAVhu.32 capsids engineered at VR4 (peptide insert), VR5 (point mutations) and VR8 (DARPin insert) or VR5 (point mutations) and VR8 (DARPin insert) compared to wildtype AAV9.

FIGS. 15A-B show that first generation DARPin inserted in AAV9 mediated up to increased transduction of HEK293-hTR cells and 293-AAVR cells compared to wtAAV9 (no DARPin) at various plasmid ratios used in manufacturing.

FIGS. 16A-B show that first generation DARPin inserted in AAV9 mediated up to increased transduction of HEK293-hTR cells and 293-AAVR cells compared to wtAAV9 (no DARPin) at various plasmid ratios used in manufacturing.

### 5. DETAILED DESCRIPTION

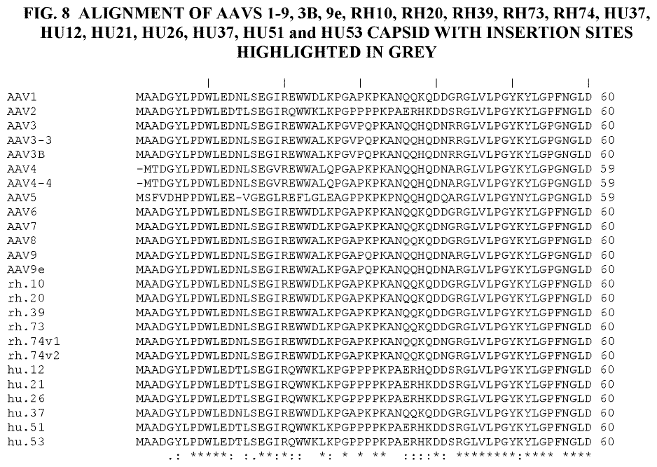

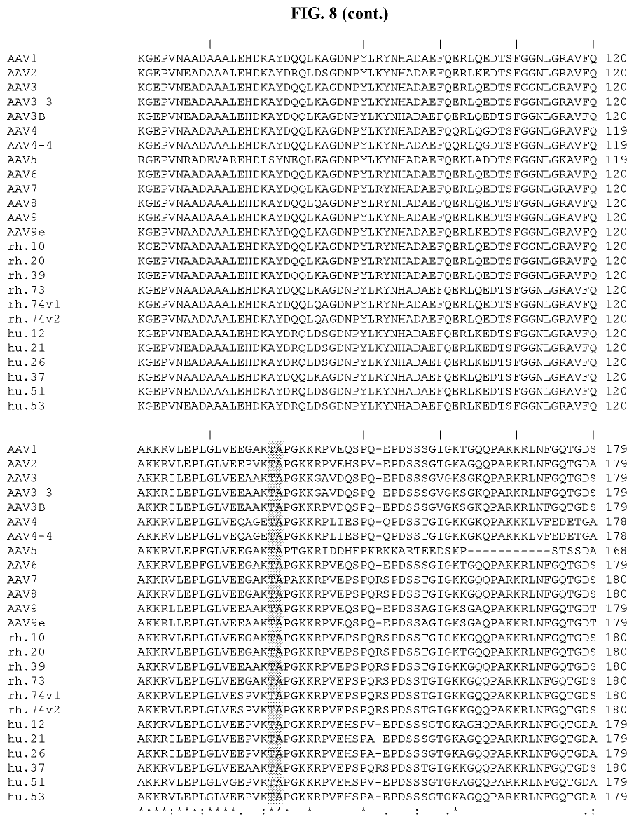

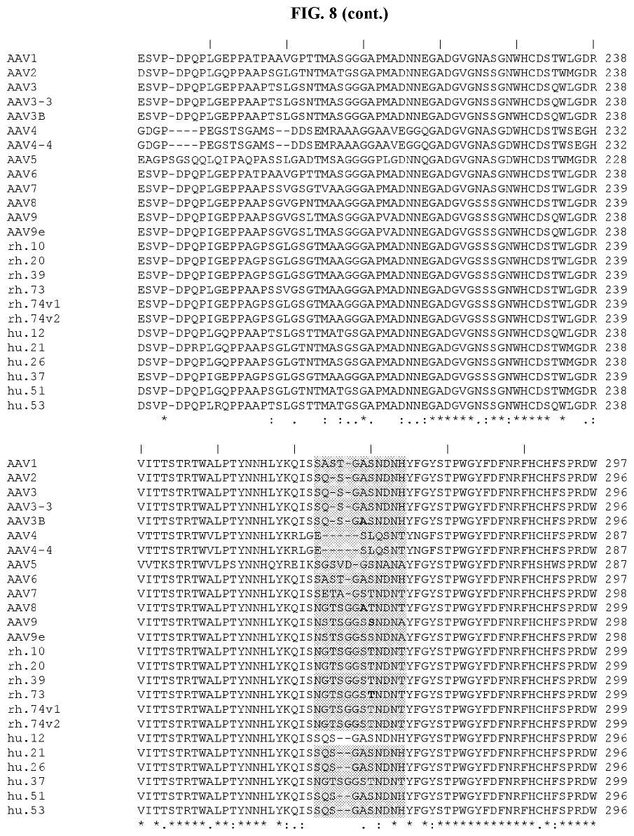

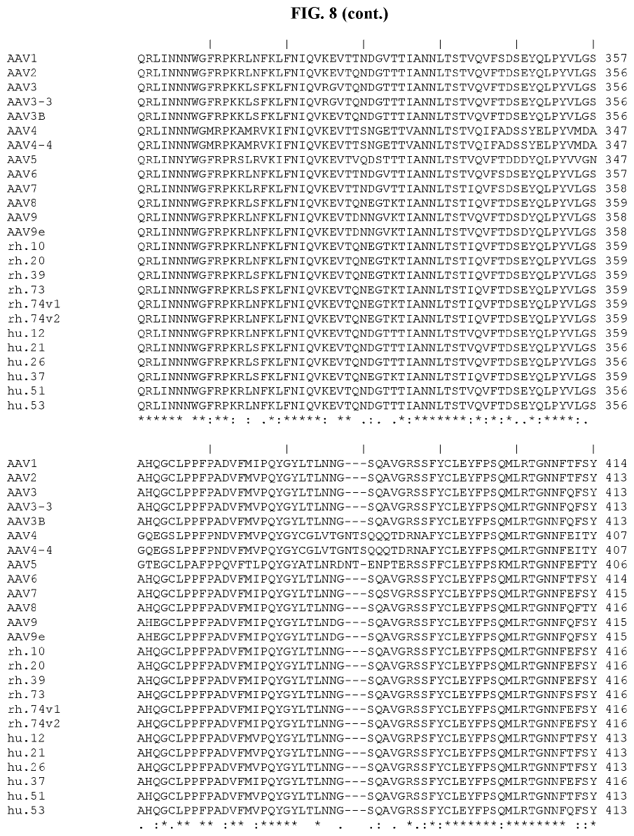

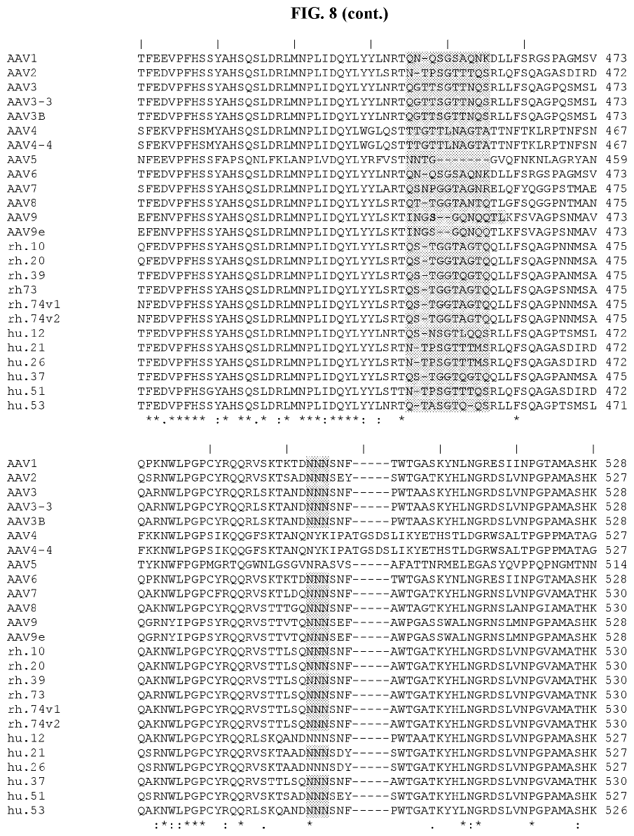

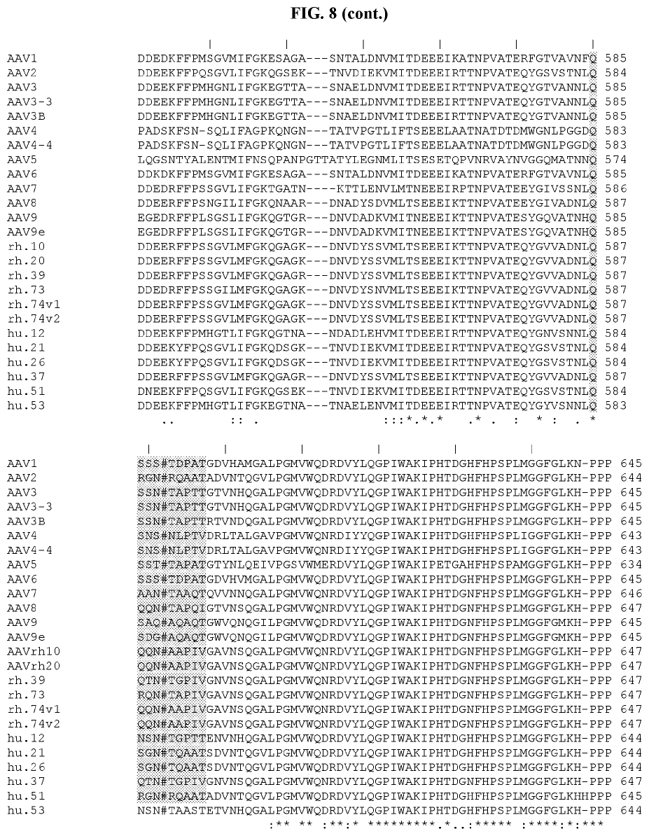

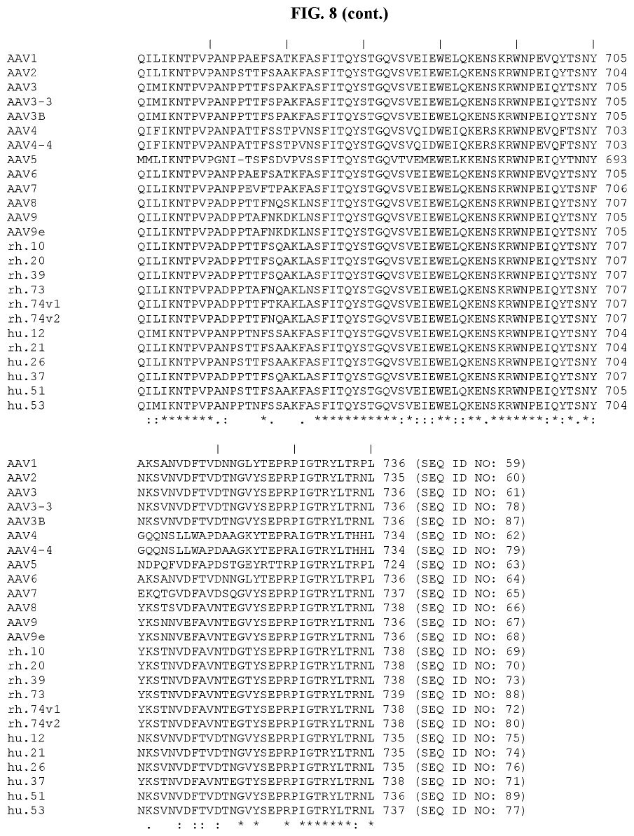

Provided are recombinant adeno-associated viruses (rAAVs) having capsid proteins engineered relative to a reference capsid protein, such that the rAAV has enhanced desired properties, such as altered tissue targeting, including transduction, genome integration and transgene expression, particularly, preferentially, relative to the reference capsid protein (e.g., the unengineered or wild type capsid), to CNS or to heart and/or skeletal muscle tissue or other tissue. In embodiments, the engineered capsid has one or more amino acid substitutions resulting in reduced tropism or atropisms (i.e., tissue targeting, transduction and integration of the rAAV genome) relative to the reference capsid (e.g., AAV9 or AAV8) for all or a subset of tissues, including one or more of heart, lung, kidney, pancreas, meniscus, liver, muscle (including biceps, transabdominal muscle, gastrocnemius muscle, and quadriceps), dorsal root ganglion and/or peripheral nervous tissue. The reduction may be a one fold. 2 fold, 5 fold, 10 fold, 20 fold, 50 fold. 100 fold, 1000 fold. 10.000 fold or even greater reduction relative to a reference capsid. The modifications include amino acid substitutions (including 1, 2, 3, 4, 5, 6, 7 or 8 amino acid substitutions), including, for AAV9, the amino acid substitutions G266A, N272A, W503R or W503A, or the corresponding amino acid substitutions for a different AAV capsid (see alignment FIG. 8) and, in embodiments, further including the amino acid substitutions 496-NNN/AAA-498 for AAV9, or the corresponding substitutions in a different AAV capsid. The AAV capsid protein to be engineered is, in certain embodiments, an AAV9 capsid protein or an AAV8 capsid protein. In other embodiments, the AAV capsid to be engineered is an AAV rh.34, AAV4, AAV5. AAV hu.26, AAV rh.31, AAV hu.13, AAV hu.56, AAV hu.53, AAV7, AAV rh64R1, AAV rh46 or AAV rh73 capsid protein. (See FIG. 8 and Table 7 for sequences and Table 8 for nucleotide sequences encoding VP1 and VP2 capsid proteins, including engineered proteins).

The engineered capsids with reduced or obviated transduction for all tissue types or one or more of heart, lung, kidney, pancreas, meniscus, liver, skeletal muscle, brain or peripheral nervous system may be further engineered to comprise a peptide insertion or attachment of another moiety, which peptide or moiety targets the rAAV to one or more tissue types, conferring tropism on the engineered capsid of the rAAV, where, for example, the peptide or moiety includes a binding domain for a receptor or other cell surface moiety characteristic of one or more tissue types. The increased targeting or transduction of tissue may be any tissue or combination of tissues, for example, skeletal muscle, heart, central nervous system, etc. In embodiments, the peptide insertion is 4 to 20, or 7 contiguous amino acids of a heterologous (not an AAV) peptide, and in embodiments no more than 12 contiguous amino acids from a heterologous protein as described herein. The targeting domain may also be an antibody or antigen binding domain thereof or other form of binding domain, such as a DARPin. The peptide, antibody, antigen binding domain thereof, DARPin or other binding or targeting domain may be inserted at an appropriate position in the VP1,VP2, and/or VP3 capsid protein, including at or near the VP2 initiation codon, or within the VR-1 region. VR-IV region, or VR-VIII region. For example, the peptide, antibody, antigen binding domain thereof, DARPin or other binding or targeting domain may be inserted at one of position 138, 262-273, 452-461, or 585-593 for AAV9 as numbered for the VP1 protein (see FIG. 8) or corresponding position for a different AAV capsid.

rAAV having an a tropic (or limited tropic) capsid comprising an insert with a targeting domain may exhibit increased transduction of one or more tissues (including skeletal muscle, heart or CNS) that is 1 fold, 2 fold. 5 fold, 10 fold. 20 fold, 50 fold, 100 fold, 500 fold, 1000 fold, 10,000 fold or 100,000 fold greater than an rAAV having the parental atropic (or limited tropic) capsid without the insert of the targeting domain.

Also provided are engineered capsids, particularly AAV9 capsids having amino acid substitutions 496-NNN/AAA-498 of AAV9 or corresponding substitutions in a different capsid type and further comprising a peptide insert of VQVGRTS (SEQ ID NO:126) inserted between S454 and G454 (herein NVG07) or inserted in another AAV capsid at a corresponding position (see, e.g., FIG. 8).

Also provided are engineered capsids, particularly AAV9 capsids having amino acid substitutions of one of G266A, N272A, W503A or W503R and the amino acid substitutions 496-NNN/AAA-498 of AAV9 or corresponding substitutions in a different capsid type and to enhance tissue specific transduction, further comprising a peptide insert of TLAAPFK (SEQ ID NO:1) inserted between Q588 and A589 (herein PHP.hDYN) or alternatively between S268 and S269 or between S454 and G455) or inserted in another AAV capsid at a corresponding position (see, e.g., FIG. 8). Or, alternatively, the capsid is modified with the amino acid substitutions of one of G266A, N272A, W503R or W503A and the amino acid substitutions 496-NNN/AAA-498 of AAV9 (or corresponding substitutions in other capsids) or AAV9 PHP.eB capsid (which has the modifications A587D and Q588G and further comprising, to enhance tissue specific transduction, insertion of the peptide TLAVPFK (SEQ ID NO:20) between G588 and A589) and the peptide TILSRSTQTG (SEQ ID NO: 15) between position 138 and 139, or the corresponding positions in other capsids (see FIG. 8 for alignment) or at any other position that displays the peptide on the capsid surface to promote tissue specific binding and transduction, for example, including at or near the VP2 initiation codon, or within the VR-1 region, VR-IV region, or VR-VIII region. Or, alternatively, the capsid is modified with the amino acid substitutions of one of G266A, N272A, W503R or W503A and the amino acid substitutions 496-NNN/AAA-498 of AAV9 (or corresponding substitutions in other capsids) and further comprising to enhance tissue specific transduction an insertion of peptide RTIGPSV (SEQ ID NO:12) between position 454 and 455, 599 and 598, or 138 and 139, or the corresponding positions in other capsids (see FIG. 8 for alignment) or at any other position that displays the peptide on the capsid surface to promote tissue specific binding and transduction, for example, including at or near the VP2 initiation codon, or within the VR-1 region, VR-IV region, or VR-VIII region. Additional capsids further have a Kidney1 peptide LPVAS (SEQ ID NO:6) inserted into the capsid, for example between S454 and G455 of AAV9, or alternatively between 5268 and 5269 or between Q588 and A589, or the corresponding position of a different capsid or at any other position that displays the peptide on the capsid surface to promote tissue specific binding and transduction, for example, including at or near the VP2 initiation codon, or within the VR-1 region. VR-IV region, or VR-VIII region. In some embodiments, the capsids can comprise R697W substitution of AAV rh64R1. The capsids having these amino acid substitutions and insertions may further have substitutions of the NNN (asparagines) at 496 to 498 with AAA (alanines) of the AAV9 capsid or at positions 498 to 500 of the AAV8 capsid, or corresponding substitutions in other AAV type capsids. Engineered capsids include AAV9.G266A.496-NNN/AAA-498 (G266A. 496-NNN/AAA-498 substitutions in the amino acid sequence of AAV9, SEQ ID NO:50), AAV9.496-NNN/AAA-498.W503R (496-NNN/AAA-498 and W503R amino acid substitutions in the amino acid sequence of AAV9, SEQ ID NO:32), or AAV9.496-NNN/AAA-498.W503A (496-NNN/AAA-498 and W503A amino acid substitutions in the amino acid sequence of AAV9, SEQ ID NO:51). In embodiments, these capsids are further modified by the insertion of a binding domain, e.g., a peptide, antibody (including a single domain antibody or other antigen binding form thereof) or DARPin at a position such that the targeting domain is displayed on the surface of the capsid when incorporated into a recombinant AAV vector, or, alternatively, a tissue specific targeting domain is created by amino acid substitutions in the capsid. In certain embodiments, transduction is measured by detection of transgene, such as the DNA of the transgene, GFP fluorescence or detection of the expressed transgene mRNA, protein or protein activity (for example, as in the examples infra). Capsids comprising a targeting domain may exhibit preferential targeting for heart and/or skeletal muscle or CNS or other tissue, and reduced targeting (compared to an AAV bearing the unengineered capsid) for liver, heart, lung, kidney, pancreas, meniscus, and/or dorsal root ganglion cells and/or peripheral nervous system tissue, and may particularly useful for delivery of a transgene encoding a therapeutic protein or nucleic acid for treatment of a muscle or CNS disease or any other disease associated with the targeted tissue. Exemplary transgenes are provided in Tables 1A and 1B.

In another embodiment, provided is a recombinant capsid protein, including an engineered AAV9 capsid protein (having the amino acid substitutions which reduce transduction of one or more tissues), and an rAAV comprising the capsid protein, in which the peptide TLAVPFK (SEQ ID NO:20) is or further comprising the peptide TLAVPFK (SEQ ID NO:20) inserted between G588 and A589 of AAV9, and, in particular, the capsid protein also has amino acid substitutions A587D/Q588G (PHP.eB) and further has the peptide TILSRSTQTG (SEQ ID NO:15) inserted after position 138 of AAV9 (collectively, AAVPHPeB.VP2Herp; see Table 7), or in the corresponding positions of another AAV. Provided are recombinant AAV capsids comprising an amino acid substitution of G266A, W503A or W503R of AAV9 (or corresponding amino acid substitution in another capsid) and further comprising insertion of the peptide RTIGPSV (SEQ ID NO:12), including inserted between positions 138 and 139, positions S454 and G455, or positions Q588 and A589 of AAV9, or corresponding position of another AAV capsid, or at any other position that displays the peptide on the capsid surface to promote tissue specific binding and transduction, for example, including at or near the VP2 initiation codon, or within the VR-1 region, VR-IV region, or VR-VIII region. Additional capsids further comprise a Kidney1 peptide LPVAS (SEQ ID NO:6) (or alternatively CLPVASC (SEQ ID NO:5)) inserted into the capsid, for example between S454 and G455 of AAV9 (see Table 7), or alternatively between S268 and S269 or between Q588 and A589, or the corresponding position of a different capsid or at any other position that displays the peptide on the capsid surface to promote tissue specific binding and transduction, for example, including at or near the VP2 initiation codon, or within the VR-1 region, VR-IV region, or VR-VIII region. Such a capsid comprising a targeting domain may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein or nucleic acid for treatment of a muscle disease (such as, but not limited to a muscular dystrophy).

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle and/or skeletal muscle cells compared to a reference AAV capsid, including an AAV9 capsid or an AAV8 capsid, or the atropic parental capsid having the amino acid substitutions which reduce or obviate rAAV transduction of one or more tissues. In particular embodiments, the muscle is gastrocnemius muscle, bicep, tricep and/or heart muscle. In further embodiments, the engineered rAAV exhibits of 50%, 60%, 70%, 80%, 90%, 95% or 99% less transduction in liver compared to the reference AAV capsid compared to a reference AAV capsid, including an AAV9 capsid or an AAV8 capsid, or the parental capsid. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid. The enhanced and/or reduce transduction may be with any mode of administration, by intravenous administration, intramuscular administration, or any type of systemic administration, intrathecal administration or ICV administration.

Also provided are engineered capsids having one or more amino acid substitutions that alter transduction and/or tissue tropism, for example, promote transduction and/or tissue tropism, particularly for enhanced, relative to an unengineered capsid (or capsid having only the amino acid substitutions which detarget the capsid from one or more tissues), targeting for CNS and, in embodiments, reduced, relative to an unengineered capsid or an atropic or reduced tropic capsid, targeting for liver, heart, skeletal muscle, lung, kidney, pancreas, meniscus, dorsal root ganglion, and/or peripheral nervous tissue. In embodiments, the amino acid substitutions are A269S of AAV8 (or at a corresponding position in a different AAV serotype capsid), S263G/S269T/A273T of AAV9 (or at a corresponding position in a different AAV serotype capsid). N272A or G266A of AAV9 (or at a corresponding position in a different AAV serotype capsid), Q474A of AAV9 (or at a corresponding position in a different AAV serotype capsid), or W503R of AAV9 (or at a corresponding position in a different AAV serotype capsid), or R697W of rh64R1 (or at a corresponding position in a different AAV serotype capsid). The capsids having these amino acid substitutions and insertions may further have or alternatively have substitutions of the NNN (asparagines) at 496 to 498 with AAA (alanines) of the AAV9 capsid (SEQ ID NO:31) or have substitutions of the NNN (asparagines) at 498 to 500 with AAA (alanines) of the AAV8 capsid, or corresponding substitutions in other AAV type capsids. Capsids comprising a targeting domain may exhibit preferential targeting for CNS, and reduced targeting (compared to an AAV bearing the unengineered capsid) for liver, heart, muscle, lung, kidney, pancreas, meniscus, and/or dorsal root ganglion cells and/or peripheral nervous system tissue, and may particularly useful for delivery of a transgene encoding a therapeutic protein or nucleic acid for treatment of a CNS disease.

Also provided are recombinant capsid proteins, and rAAVs comprising them, that have inserted peptides that target and/or promote rAAV cellular uptake, transduction and/or genome integration in CNS tissue and, in embodiments, reduced, relative to an unengineered capsid (or atropic capsid), targeting for liver, heart, muscle, lung, kidney, pancreas, meniscus, dorsal root ganglion, and/or peripheral nervous tissue, for example, the peptide TILSRSTQTG (SEQ ID NO:15); TLAVPFK (SEQ ID NO:20); or TLAAPFK (SEQ ID NO:1). In particular embodiments the peptide TLAAPFK (SEQ ID NO:1) is inserted between Q588 and A589 of AAV9 (AAV9.hDyn; see Table 7), or the corresponding position of another AAV (see FIG. 8). Alternatively, the capsid is rh.34, rh.10, rh.46, rh.73, or rh64.R1 (FIG. 8 or Table 7 for sequence), or an engineered form of rh.34, rh.10, rh.46, rh.73, or rh64.R1. The insertions may also be in an atropic or reduced tropic parental capsid which has an amino acid substitution of G266A, N272A, W503R, or W503A and amino acid substitutions of 496 NNN/AAA 498 of AAV9 or corresponding substitutions in a different capsid serotype. These engineered capsids may exhibit preferential targeting for CNS, and reduced targeting (compared to an AAV bearing the unengineered capsid) for liver, heart, muscle, lung, kidney, pancreas, meniscus, and/or dorsal root ganglion cells and/or peripheral nervous system tissue, and may particularly useful for delivery of a transgene encoding a therapeutic protein or nucleic acid for treatment of a CNS disease.

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction in CNS tissue compared to a reference AAV capsid, such as the parental capsid (including an atropic or reduced tropic capsid) or AAV8 or AAV9 or AAVhu32. The CNS tissue may be one or more of the frontal cortex, hippocampus, cerebellum, midbrain and/or hindbrain. In further embodiments, the engineered rAAV exhibits of 50%, 60%, 70%, 80%, 90%, 95% or 99% less transduction in liver compared to the reference AAV capsid such as the parental capsid or AAV8 or AAV9 or AAVhu32. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid such as the parental capsid or AAV8 or AAV9 or AAVhu32. The enhanced and/or reduce transduction may be with any mode of administration, by intravenous administration, intramuscular administration, or any type of systemic administration, intrathecal administration or ICV administration.

Recombinant vectors comprising the capsid proteins also are provided, along with pharmaceutical compositions thereof, nucleic acids encoding the capsid proteins, and methods of making and using the capsid proteins and rAAV vectors having the engineered capsids for targeted delivery, improved transduction and/or treatment of disorders associated with the target tissue.

As used throughout, AAV “serotype” refers to an AAV having an immunologically distinct capsid, a naturally-occurring capsid, or an engineered capsid.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3. Unless otherwise specified, the numbering for capsid proteins is for VP1 as shown in FIG. 8. For VP2 proteins, the amino acid position may be that corresponding to that amino acid as numbered according to the VP1 sequence as shown in FIG. 8 but may be identified by a different amino acid position number due to the difference in the start of the N-terminus.

The term “rep gene” refers to the nucleic acid sequences that encode the non-structural protein needed for replication and production of virus.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the term “designed ankyrin repeat protein” or “DARPin” refers to a non-natural protein comprising an ankyrin repeat domain. In embodiments, a DARPin has a repeat sequence motif that was derived from natural ankyrin repeats, e.g. by consensus design (see, e.g., Forrer et al., 2004 Chem Bio Chem, 5, 2, 183-189 and Binz, H. K., et al., J. Mol. Biol., 332, 489-503, 2003). Exemplary DARPins include, but are not limited to, DARPins found in U.S. Pat. No. 11,242,369 and WO 2023/021050, incorporated by reference in their entireties. Also contemplated are DARPins identified in DARPin libraries, such as described in U.S. Ser. No. 10/457,717, EP10646542, US20180163229A1, WO2018152326A1, WO2020245171A1 and WO2021116462A1 (each of which are hereby incorporated by reference in their entireties). DARPin inserts may be about 60, 70, 80, 90, 100, 110, 120, 130, 140, 150, 160, 170, 180, 190 or more amino acids in length, or even more than 200 amino acids in length.

As used herein, the terms “subject”. “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

As used herein, the terms “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The “central nervous system” (“CNS”) as used herein refers to neural tissue reaches by a circulating agent after crossing a blood-brain barrier, and includes, for example, the brain, optic nerves, cranial nerves, and spinal cord. The CNS also includes the cerebrospinal fluid, which fills the central canal of the spinal cord as well as the ventricles of the brain.

### 5.2. Recombinant AAV Capsids and Vectors

Provided are recombinant adeno-associated viruses (rAAVs) having capsid proteins engineered relative to a reference capsid protein, such that the rAAV has enhanced desired properties, such as altered tissue targeting, including transduction, genome integration and transgene expression, particularly, preferentially, relative to the reference capsid protein (e.g., the unengineered or wild type capsid), to CNS or to heart and/or skeletal muscle tissue or any other type of tissue. Provided are rAAV proteins comprising an insertion of a DARPin, wherein the DARPin targets a cell surface molecule. In embodiments, the DARPin is flanked on either one side or both sides by a linker. In embodiments, the DARPin is inserted into a wild type capsid protein. In embodiments, the DARPin is inserted into VR4 or VR8 of either VP1, VP2 and/or VP3.

In embodiments, the rAAV capsid protein comprising the DARPin insert is a VP1 protein or a VP2 protein or VP3 protein. In embodiments, a two plasmid system is used to make engineered recombinant rAAV capsids comprising VP1, VP2, and/or VP3 with distinct targeting and/or detargeting mutations. In embodiments, the engineered capsids comprise 1) a VP1 and VP3 protein comprising a substitution and/or insertion that detargets the capsid and a VP2, also optionally comprising the substitution and/or insertion that detargets the capsid, and comprising a DARPin insert, or 2) a VP1 and VP3 that do not comprise a substitution or insertion (e.g., a wild type VP1 and VP3 protein) and a VP2 comprising a DARPin insert, or 3) a VP2 and VP3 that do not comprise a substitution or insertion and a VP1 comprising a DARPin insert, or 4) a VP2 and VP3 protein comprising a substitution and/or insertion that detargets the capsid and a VP1, also optionally comprising the substitution and/or insertion that detargets the capsid, and comprising a substitution and/or insertion that detargets the capsid and comprises a DARPin insert or 5) a VP1 and VP2 protein comprising a substitution and/or insertion that detargets the capsid and a VP3, optionally comprising a substitution and/or insertion that detargets the capsid, and comprising a DARPin insert, or 6) a VP1 and VP2 protein that do not comprise a substitution or insertion (e.g., a wild type VP1 and VP3 protein) and a VP3 comprising a DARPin insert. Capsids having combinations of VP1, VP2 and VP3 proteins with one or two of VP1, VP2, or VP3 comprising a DARPin insert may be produced by transfecting producer host cells with polynucleotides that encode the VP1, VP2 and/or VP3 protein having the DARPin insert and VP1, VP2, and/or VP3 proteins without the DARPin insert, including, for example, inactivating of the start codon for one of VP1, VP2 and/or VP3 such that the construct expresses a subset of the VP1, VP2 and/or VP3 proteins not having the DARPin insert or expressing all three VP1, VP2 and/or VP3 protein.

Provided are recombinant AAV capsid proteins comprising an insertion of a DARPin which targets a cell surface molecule and wherein the DARPin is flanked on at least one end by a linker, wherein a recombinant AAV particle incorporating the recombinant AAV capsid protein has cell transduction activity. Cell transduction activity can be measured by any method known in the art, including measuring GFP fluorescence as shown in Example 2.

In embodiments, the rAAV capsid protein is an AAV9 or AAVhu32 capsid protein and comprises no other substitutions or insertions. In embodiments, the rAAV capsid protein further comprises one or more amino acid substitutions and/or insertions relative to the wild type or unengineered capsid protein which when incorporated into an rAAV capsid exhibits reduced transduction or exhibits increased transduction of at least one tissue type relative to an rAAV capsid incorporating the wild type or unengineered capsid protein.

In embodiments, the rAAV capsid protein has (1) a G266A substitution, a N272A substitution, a W503A substitution or a VQVGRTS insertion between 454 and 455 or (2) 496-NNN/AAA-498 substitutions, or (3) a combination thereof, for an AAV9 capsid protein, or corresponding substitutions in a capsid protein of another AAV type capsid. In embodiments, the rAAV capsid protein comprises N272A and 496-NNN/AAA-498 substitutions. In embodiments, the rAAV capsid protein comprises an insertion of the DARPin is within the VR-1 region. VR-IV region, or VR-VIII region of the VP1, VP2, and/or VP3 protein.

In embodiments, the DARPin insertion is at one of positions 138, 262-273, 452-461, or 585-593, or replaces amino acids 452-461 or 585-593, for AAV9 as numbered for the VP1 amino acid sequence or corresponding position for a different AAV capsid. In embodiments, the DARPin insertion is between Q588 and A589. S268 and S269, before or after 1451, N452, G453, S454, G455, Q456. N457, Q458. Q459, T460, or L461 of AAV9 as numbered for the VP1 amino acid sequence or corresponding position of a different AAV capsid protein. In embodiments, the DARPin replaces one or more of amino acids 452-461 or 585-593 of AAV9 as numbered for the VP1 amino acid sequence or corresponding amino acids of a different AAV capsid protein.

In embodiments, the DARPin is flanked on at least one end by a linker. In embodiments, the linker comprises a (G).S linker, where n=2-10. In embodiments, the linker is a GS linker. In embodiments, the linker is a PT linker. In embodiments, the linker comprises GGS, GGGGS (SEQ ID NO: 127), 4GSx2 (SEQ ID NO: 135), 4GSx3 (SEQ ID NO: 136), 4GSx4 (SEQ ID NO: 137). GS-4GSx4-GS (SEQ ID NO: 138), GS-PT linker-GS (SEQ ID NO: 139) or GS-PT linker-GS (SEQ ID NO: 140). In embodiments, the linker is at the N-terminal side of the DARPin insert. In embodiments, the linker is at the C-terminal side of the DARPin insert. In embodiments, the linker is at both the N-terminus and C-terminus of the DARPin insert.

In embodiments, the DARPin targets a CNS cell surface protein. In embodiments, when a VP1, VP2 and/or VP3 protein comprising the DARPin insert is incorporated into a rAAV particle, the rAAV particle has increased targeting, binding or transduction into CNS cells, relative to a rAAV particle incorporating the corresponding capsid protein without the DARPin insertion. In embodiments, the DARPin targets a human receptor or other cell surface protein, including one that is expressed on CNS cells, including on certain types of CNS cells, or GluA4. In embodiments, the DARPin targets a human receptor or GluA4.

In embodiments, the DARPin targets an ocular cell surface protein. In embodiments, when a VP1, VP2 and/or VP3 protein comprising the DARPin insert is incorporated into a rAAV particle, the rAAV particle has increased targeting, binding or transduction into ocular cells, relative to a rAAV particle incorporating the corresponding capsid protein without the DARPin insertion. In embodiments, the DARPin targets a human receptor or other cell surface protein, including one that is expressed on ocular cells, including on certain types of ocular cells.

In embodiments, the DARPin targets a muscle cell surface protein. In embodiments, when a VP1. VP2 and/or VP3 protein comprising the DARPin insert is incorporated into a rAAV particle, the rAAV particle has increased targeting, binding or transduction into muscle cells, relative to a rAAV particle incorporating the corresponding capsid protein without the DARPin insertion. In embodiments, the DARPin targets a human receptor or other cell surface protein, including one that is expressed on muscle cells, including on certain types of muscle cells.

In embodiments, the DARPin is inserted into a capsid protein that has one or more amino acid substitutions and/or insertions relative to a wild-type or unengineered capsid protein. In embodiments, the one or more amino acid substitutions and/or insertions result in reduced tropism or atropism (i.e., tissue targeting, transduction and integration of the rAAV genome) relative to the reference capsid (e.g., AAV9 or AAV8) for all or a subset of tissues, including one or more of heart, lung, kidney, pancreas, meniscus, liver, muscle (including biceps, transabdominal muscle, gastrocnemius muscle, and quadriceps), brain, dorsal root ganglion and/or peripheral nervous tissue. The reduction may be a one fold, 2 fold, 5 fold, 10 fold, 20 fold, 50 fold, 100 fold, 1000 fold, 10,000 fold or even greater reduction relative to a reference capsid. The modifications include amino acid substitutions (including 1, 2, 3, 4, 5, 6, 7 or 8 amino acid substitutions), including, for AAV9, the amino acid substitutions G266A, N272A, W503R or W503A, or the corresponding amino acid substitutions for a different AAV capsid (see alignment FIG. 8) and, in embodiments, further including the amino acid substitutions 496-NNN/AAA-498 for AAV9, or the corresponding substitutions in a different AAV capsid. The atropic or reduced tropic capsids may be further modified to confer a specific tissue tropism, for example, the rAAV incorporating the modified capsids have increased transduction of CNS, skeletal muscle, heart or any other tissue by incorporation of a targeting domain, such as a peptide, antibody or antigen binding domain or a DARPin. The modifications include amino acid substitutions (including 1, 2, 3, 4, 5, 6, 7 or 8 amino acid substitutions) and/or peptide insertions (4 to 20, or 7 contiguous amino acids, and in embodiments no more than 12 contiguous amino acids from a heterologous protein) or insertions of longer polypeptides such as antigen binding domains of an antibody or a DARPin domain, as described herein at positions within the capsid protein such that the peptide, antibody or other binding domain is displayed on the capsid surface when the capsid protein is incorporated into a recombinant AAV vector and can bind to the target tissue and/or promote transduction of the target tissue by the rAAV.

5.2.1 Engineered Capsids with Amino Acid Substitutions

In some embodiments, AAV capsids were modified by introducing selected single to multiple amino acid substitutions which reduce the transduction of vectors incorporating the AAV capsids to one or more tissue types, including liver, heart, muscle, brain, lung, kidney, pancreas, meniscus, and/or muscle to generate atropic or reduced tropic capsids and/or increase effective gene delivery to the CNS or to cardiac or skeletal muscle or other target tissue (including when the atropic or limited tropic capsids incorporate a targeting domain to enhance tropism to CNS or to cardiac or skeletal muscle or other target tissue), detarget the liver and/or dorsal root ganglion to reduce toxicity, and/or reduce immune responses of neutralizing antibodies.

In particular embodiments the capsids have one or more amino acid substitutions including a W503A or W503R substitution, a Q474 substitution, a N272A or N266A substitution in AAV9 or the corresponding substitution in another AAV serotype or an A269S substitution in AAV8 or the corresponding substitution in another AAV serotype. rAAV having a capsid with the Q474A substitution may be particularly useful for delivery to skeletal and/or cardiac muscle or CNS tissue and rAAV having a capsid with the W503R substitution may be particularly useful for delivery to CNS tissue, particularly with reduced, compared to reference capsid containing rAAVs, transduction in the liver and/or DRGs. Other substitutions include S263G/S269R/A273T substitutions in AAV9 or A587D/Q588G in AAV9 or corresponding substitutions in other AAV serotypes. In some embodiments, the rAAV capsid can have a R697W substitution. The capsids having these amino acid substitutions and insertions may further have substitutions of the NNN (asparagines) at 496 to 498 with AAA (alanines) of the AAV9 capsid, or of the NNN (asparagines) at 498 to 500 with AAA (alanines) of the AAV8 capsid corresponding substitutions in other AAV type capsids. Other AAV serotypes that may be used for the amino acid substitutions and that may be the reference capsid include AAV8, AAV rh.34, AAV4, AAV5, AAV hu.26, AAV rh.31, AAV hu.13, AAV hu.26, AAV hu.56, AAV hu.53, AAV7, rh64R1, rh46 or rh73. In particular embodiments for CNS delivery, the capsid is rh34, either unmodified or serving as the parental capsid to be modified as detailed herein.

Provided are atropic capsids which may be further be modified to confer specific tissue tropism and/or enhanced tissue-specific transduction by inserting a targeting domain (or introducing one or more amino acid substitutions which create a tissue specific binding domain within the capsid). Such atropic capsids include AAV9 and other capsids comprising or consisting of an amino acid substitution of G266A. N272A, W503R or W503A for AAV9, or the corresponding amino acid substitutions for a different AAV capsid (see alignment FIG. 8) and the amino acid substitutions 496-NNN/AAA-498 for AAV9, or the corresponding substitutions. rAAV incorporating these capsids exhibit reduced tissue targeting and transduction in heart, lung, kidney, pancreas, meniscus, liver, and muscle (including biceps, transabdominal muscle, gastrocnemius muscle, and quadriceps), and may exhibit reduced transduction in dorsal root ganglion and/or peripheral nervous tissue. The reduction may be a one fold, 2 fold, 5 fold, 10 fold, 20 fold, 50 fold, 100 fold, 1000 fold, 10,000 fold or even greater reduction relative to a reference capsid (see, e.g., Examples 19 and 20). Specific atropic capsids disclosed herein include AAV9.G266A.496NNN/AAA498 (SEQ ID NO:50), AN272A.496NNN/AAA498 (SEQ ID NO:49), AAV9.496NNN/AAA498.W503R (SEQ ID NO:32), and AAV9.496NNN/AAA498.W503A (SEQ ID NO:51).

Effective gene delivery to the CNS by intravenously administered rAAV vectors requires crossing the blood brain barrier. Key clusters of residues on the AAVrh.10 capsid that enabled transport across the brain vasculature and widespread neuronal transduction in mice have recently been reported. Specifically, AAVrh.10-derived amino acids N262, G263, T264, S265, G267, S268. T269, and T273 were identified as key residues that promote crossing the BBB (Albright et al, 2018. Mapping the Structural Determinants Required for AAVrh.10 Transport across the Blood-Brain Barrier). Amino acid substitutions in capsids, such as AAV8 and AAV9 capsids that promote rAAV crossing of the blood brain barrier, transduction, detargeting of the liver and/or reduction in immune responses have been identified.

In some embodiments, provided are capsids having one or more amino acid substitutions that promote transduction and/or tissue tropism of the rAAV having the modified capsid. In particular embodiments, provided are capsids having a single mutation at amino acid 269 of the AAV8 capsid replacing alanine with serine (A269S) (see, Tables 5a-5c, herein referred to as AAV8.BBB) and amino acid substitutions at corresponding positions in other AAV types. In some embodiments, provided are capsids having multiple substitutions at amino acids 263, 269, and 273 of the AAV9 capsid resulting in the following substitutions: S263G, S269T, and A273T (herein referred to as AAV9.BBB) or substitutions corresponding to these positions in other AAV types. These amino acid substitutions may be incorporated into the atropic capsids described herein (including, for example, AAV9.G266A.496NNN/AAA498 (SEQ ID NO:50), AAV9.N272A.496NNN/AAA498 (SEQ ID NO:49), AAV9.496NNN/AAA498.W503R (SEQ ID NO:32), and AAV9.496NNN/AAA498.W503A (SEQ ID NO:51)).

Exposure to the AAV capsid can generate an immune response of neutralizing antibodies. One approach to overcome this response is to map the AAV-specific neutralizing epitopes and rationally design an AAV capsid able to evade neutralization. A monoclonal antibody, specific for intact AAV9 capsids, with high neutralizing titer has recently been described (Giles et al, 2018, Mapping an Adeno-associated Virus 9-Specific Neutralizing Epitope To Develop Next-Generation Gene Delivery Vectors). The epitope was mapped to the 3-fold axis of symmetry on the capsid, specifically to residues 496-NNN-498 and 588-QAQAQT-592 of AAV9 (SEQ ID NO:8). Capsid mutagenesis demonstrated that single amino acid substitution within this epitope markedly reduced binding and neutralization. In addition, in vivo studies showed that mutations in the epitope conferred a “liver-detargeting” phenotype to the mutant vectors, suggesting that the same residues are also responsible for AAV9 tropism. Liver detargeting has also been associated with substitution of amino acid 503 replacing tryptophan with arginine. Presence of the W503R mutation in the AAV9 capsid was associated with low glycan binding avidity (Shen et al, 2012, Glycan Binding Avidity Determines the Systemic Fate of Adeno-Associated Virus Type 9).

In some embodiments, provided are capsids in which the AAV8.BBB and AAV9.BBB capsids were further modified by substituting asparagines at amino acid positions 498, 499, and 500 of AAV8 (herein referred to as AAV8.BBB.LD) or 496, 497, and 498 of AAV9 (herein referred to as AAV9.BBB.LD) with alanines. In some embodiments, the AAVrh10 capsid was modified by substituting three asparagines at amino acid positions 498, 499, and 500 to alanines (AAVrh10.LD) (Tables 5a-5c).

In some embodiments, provided are capsids having three asparagines at amino acid positions 496, 497, and 498 of the AAV9 capsid replaced with alanines and also tryptophan at amino acid 503 of the AAV9 capsid with alanine or arginine or capsids with substitutions corresponding to these positions in other AAV types. In some embodiments, provided are capsids having glutamine at amino acid position 474 of the AAV9 capsid substituted with alanine or capsids with substitutions corresponding to this position in other AAV types.

In some embodiments, the capsid is an AAV8.BB.LD capsid (A269S,498-NNN/AAA-500 substitutions in the amino acid sequence of AAV8), an AAV9.BBB.LD capsid (S263G/S269T/A273T, 496-NNN/AAA-498 substitutions in the amino acid sequence of AAV9), an AAV9.496-NNN/AAA-498 capsid (SEQ ID NO:31), an AAV9.496-NNN/AAA-498.W503R capsid (SEQ ID NO:32), an AAV9.W503R capsid (SEQ ID NO:33), or an AAV9.Q474A capsid (SEQ ID NO:34). In other examples, the capsid can be an AAV9.N272A.496-NNN/AAA-498 capsid (SEQ ID NO:49) or an AAV9.G266A.496NNN/AAA498 capsid (SEQ ID NO:50), or AAV9.496NNN/AAA498.W503A (SEQ ID NO:51).

In some embodiments, the rAAVs described herein (including those with atropic or limited tropic capsids having a tissue targeting domain inserted therein) increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution and/or targeting domain insertion (including relative to parental atropic capsids). In some embodiments, the increase in tissue specific cell transduction is at least 2, 10, 20, 30, 40, 50, 60, 70, 80, 90, or 100 fold more than that without the modification, i.e., relative to the parental capsid, including an atropic or limited tropic capsid. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type in a subject (a human, non-human primate or mouse subject) or in cell culture compared to the rAAV (e.g., the parental atropic or limited tropic AAV capsid) not comprising the peptide insertion. In some embodiments, the increase in genome integration is at least 2, 10, 20, 30, 40, 50, 60, 70, 80, 90, or 100 fold more than an AAV having a capsid without the modification (i.e., the parental capsid). For example, in some embodiments, there is a 50-80 fold increase in genome integration compared to genome integration with the same AAV type without the modification.

5.2.2 rAAV Vectors with Recombinant Protein Insertions

Provided are capsids with large insertions of recombinant proteins not limited to ankyrin repeat proteins (DARPins), antibodies or antigen-binding fragment, enzymes, fluorescent proteins, ligands, receptors or receptor fusion protein.

There exist examples of natural proteins contain tandem repeats modules of highly conserved amino acids. This structure acts as a building block that forms the underlying structure of certain protein-binding interactions. Among these structures and one of the most frequently observed motifs is ankyrin repeats, which is the basis for recombinant proteins named “designed ankyrin repeat proteins” (DARPins). DARPins typically consist of several repeats of an approximately 33 amino acid residue motif that is highly conserved. DARPins are known to have an elongated, rod-like shape structure with termini at opposing ends. The amino acid repeats form a 0-turn followed by two antiparallel a-helices, such that multiple repeats stack together form a scaffold. This scaffold lends itself to presentation of multiple functional and/or recognition domains, such as binding domains, thus forming a multivalent binding moiety. Thus DARPins (much like recombinant antibodies) can be synthesized for use in a wide range of functions, including protein-protein interactions, such as binding to a particular receptor or catalytic domain of an enzyme, or binding to a protein or proteins for assembly of stable multiprotein complexes (Hollenbeck, et al., Biomacromolecules. 2012 Jul. 9; 13(7): 1996-2002; Tyrkalska, et al., 2017 Front. Immunol. 8:1375). Long protein insertions may comprise DARPins having two, three, four, five, six, seven or more repeats. Thus the DARPin insert is about 60, 70, 80, 90, 100, 110, 120, 130, 140, 150, 160, 170, 180, 190 or more amino acids in length, or even more than 200 amino acids in length.

Other recombinant proteins are useful to display on the cell surface of a viral capsid protein in order to redirect the tropism or enhance transduction of the viral vector. Provided are rAAV vectors comprising engineered capsids, and a method of making such rAAVs, wherein the capsids are engineered to target cells or cell membrane receptors of interest and when inserted into the capsid protein have been shown to re-direct the rAAV to the cell or tissue of interest. Long sequence insertions in the capsids, such as full or partial proteins are advantageous compared to short peptides when inserted into a capsid since the heterologous protein's function will be retained (Morizono et al., 2005, “Lentiviral vector retargeting to P-glycoprotein on metastatic melanoma through intravenous injection” Nature medicine 11(3): 346-352; Kasaraneni et al., 2017, “Retargeting lentiviruses via Spy Catcher-SpyTag chemistry for gene delivery into specific cell types” MBio 8(6): 10-1128; Kasaraneni et al., 2018, “A simple strategy for retargeting lentiviral vectors to desired cell types via a disulfide-bond-forming protein-peptide pair” Scientific Reports 8(1): 10990).

The recombinant protein insertion is encoded by a nucleotide sequence inserted into a capsid sequence on a packaging plasmid or integrated into the genome of a packaging cell. The nucleotide sequence insertion encodes a protein, an antibody or an antigen-binding fragment (such as a Fab, Fab′(2)′, scFv, scFv-Fc, diabody, single-domain antibody, etc.), a nanobody, a designed ankyrin repeat protein (DARPin), a receptor ligand molecule, a receptor, a receptor fusion protein, a growth factor, a hormone, or any protein binding domain thereof.

Recombinant protein insertions may have ligand or receptor targeting and thus bind to the target ligand or receptor of interest. Suitable targets may be cell surface receptors or ligands that bind to cell surface receptors such that a capsid (vector) will be targeted to (bind to) that cell. The target of the insertion, e.g. DARPin insertion, is selected from, but is not limited to, a cytokine (such as VEGF, TNFα, IFNγ, IL-2, IL-6, etc.), a cytokine receptor, an integrin receptor, a transferrin ligand, a transferrin receptor, a hormone, a hormone receptor, a neuronal receptor, a neuropeptide, a neurotransmitter, a neurotransmitter receptor, a growth factor, a glutamate receptor (or receptor subunit such as GluA4), a G protein-coupled receptor, a tyrosine kinase receptor (such as HER2), or the like. DARPins may also function as antagonists or agonists and modulate cell signaling upon interaction with a ligand or cell or cell surface receptor (Stumpp, et al. Drug Discovery Today, August 2008, 13(15-16):695-701; Shilova and Deyev, Acta Naturae. 2019 October-December; 11(4): 42-53; Hartmann, et al., Molecular Therapy: Methods & Clinical Development, Vol. 10 Sep. 2018). In some examples, the DARPin insertion was used as the targeting ligand that binds to the glutamate receptor subunit GluA4.

5.2.3 rAAV Vectors with Additional Peptide Insertions

Provided are rAAVs having capsid proteins with one or more (generally one or two) peptide insertions wherein the peptide insertion increase effective gene delivery to the CNS or to cardiac or skeletal muscle and to detarget the liver and/or dorsal root ganglion to reduce toxicity relative to the parental capsid protein. The peptide may be a DARPin or may be a shorter 4, 5, 6, 7, 8, 9, or 10 mer insertion. In embodiments, the capsid protein may comprise an insertion of a DARPin and an insertion of a shorter 4, 5, 6, 7, 8, 9, or 10 mer insertion, both of which affect the tropism of the assembled capsid. In particular embodiments, the peptides include TLAVPFK (SEQ ID NO:20), TLAAPFK (SEQ ID NO:1), or TILSRSTQTG (SEQ ID NO: 15) (or an at least 4, 5, 6, 7 amino acid portion thereof). The peptides may be inserted into the AAV9 capsid, for example after the positions 138; 262-273; 452-461; 585-593 of AAV9 cap, particularly after position 138, 454 or 588 of AAV9 or a corresponding position in another AAV as detailed herein. In particular embodiments, the capsid has the peptide TLAVPFK (SEQ ID NO:20) is inserted between G588 and A589 of AAV9, and, in particular, the capsid protein also has amino acid substitutions A587D/Q588G (PHP.eB) and further has the peptide TILSRSTQTG (SEQ ID NO:15) inserted after position 138 of AAV9 (collectively, AAVPHPeB.VP2Herp; see Table 7), or in the corresponding positions of another AAV. Additional capsids have a Kidney1 peptide LPVAS (SEQ ID NO:6) inserted into the capsid, for example between 454 and 455 of AAV9 (see Table 7), or alternatively or alternatively between 5268 and S269 or between Q588 and A589 of AAV9 or the corresponding position of another AAV serotype. Such an engineered capsid may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein or nucleic acid for treatment of a muscle disease (such as, but not limited to a muscular dystrophy).

In some embodiments, the peptide insertion comprises at least 4, 5, 6, 7, 8, 9, or all 10 consecutive amino acids of sequence TILSRSTQTG (SEQ ID NO: 15), preferably which contains the TQT or STQT (SEQ ID NO:9) motif. In some embodiments, the peptide insertion consists of at least 4, 5, 6, 7, 8, 9, or all 10 consecutive amino acids of sequence TILSRSTQTG (SEQ ID NO:15), preferably which contains the TQT or STQT (SEQ ID NO:9) motif.

In certain embodiments, the peptide insertion may be a sequence of consecutive amino acids from a domain that targets kidney tissue, or a conformation analog designed to mimic the three-dimensional structure of said domain. In some embodiments, the kidney-homing domain comprises the sequence CLPVASC (SEQ ID NO:5) (see, e.g., U.S. Pat. No. 5,622,699). In some embodiments, the peptide insertion from said kidney-homing domain comprises at least 4, 5, 6, or all 7 amino acids from sequence CLPVASC (SEQ ID NO:5). In some embodiments, the peptide insertion comprises or consists of the sequence CLPVASC (SEQ ID NO:5).

It has been found that both of the cysteine residues in certain homing peptides can be deleted without significantly affecting the organ homing activity of the peptide. For example, a peptide having the sequence LPVAS (SEQ ID NO:6) also can be a kidney-homing peptide. Methods for determining the necessity of a cysteine residue or of amino acid residues N-terminal or C-terminal to a cysteine residue for organ homing activity of a peptide are routine and well known in the art. Thus, in some embodiments, the peptide insertion comprises at least 4 or all 5 amino acids from sequence LPVAS (SEQ ID NO:6). In some embodiments, the peptide insertion comprises or consists of the sequence LPVAS (SEQ ID NO:6).

In particular embodiments, provided are rAAVs having a capsid that has the peptide TLAAPFK (SEQ ID NO:1) is inserted between Q588 and A589 of AAV9 (AAV9.hDyn; see Table 4a), or the corresponding position of another AAV (see, e.g., FIG. 8). Such an engineered capsid may exhibit preferential targeting for CNS tissue, and reduced targeting (as compared to an AAV having the unengineered capsid) for liver and/or dorsal root ganglion cells and may particularly useful for delivery of a transgene encoding a therapeutic protein or nucleic acid for treatment of a CNS disease.

### 5.2.4 AAV Capsid Insertion Sites

Provided are capsids with large polypeptide or protein (DARPin) and optionally with peptide insertions at positions amenable to insertions within and near the AAV9 capsid VR-IV loop and corresponding regions on the VR-IV loop of capsids of other AAV types. Though previous studies analyzed potential positions in various AAVs, none identified the AAV9 VR-IV as amenable for this purpose (consider, e.g., Wu et al, 2000, “Mutational Analysis of the Adeno-Associated Virus Type 2 (AAV2) Capsid Gene and Construction of AAV2 Vectors with Altered Tropism,” J of Virology 74(18):8635-8647; Lochrie et al. 2006, “Adeno-associated virus (AAV) capsid genes isolated from rat and mouse liver genomic DNA define two new AAV species distantly related to AAV-5,” Virology 353:68-82; Shi and Bartlett, 2003, “RGD Inclusion in VP3 Provides Adeno-Associated Virus Type 2 (AAV2)-Based Vectors with a Heparan Sulfate-Independent Cell Entry Mechanism,” Molecular Therapy 7(4):515525-; Nicklin et al., 2001, “Efficient and Selective AAV2-Mediated Gene Transfer Directed to Human Vascular Endothelial Cells” Molecular Therapy 4(2):174-181: Grifman et al., 2001, “Incorporation of Tumor-Targeting Peptides into Recombinant Adeno-associated Virus Capsids,” Molecular Therapy 3(6):964-975: Girod et al. 1999, “Genetic capsid modifications allow efficient re-targeting of adeno-associated virus type 2,” Nature Medicine 3(9):1052-1056: Douar et al., 2003, “Deleterious effect of peptide insertions in a permissive site of the AAV2 capsid, “Virology 309:203-208; and Ponnazhagan, et al. 2001, J. of Virology 75(19):9493-9501).

Accordingly, provided are rAAV vectors carrying DARPin or peptide insertions at these points, in particular, within surface-exposed variable regions in the capsid coat, particularly within or near the variable region IV of the capsid protein. In some embodiments, the rAAV capsid protein comprises a peptide insertion immediately after (i.e., connected by a peptide bond C-terminal to) an amino acid residue corresponding to one of amino acids 451 to 461 of AAV9 capsid protein (amino acid sequence SEQ ID NO:74 and see FIG. 8 for alignment of capsid protein amino acid sequence of other AAV serotypes with amino acid sequence of the AAV9 capsid and Tables 4a, 5a, 5b, and 5c and Table 7 for other capsid sequences), where said peptide insertion is surface exposed when the capsid protein is packaged as an AAV particle. In other embodiments, the insertion is at or near the VP2 initiation codon, or within the VR-1 region, VR-IV region, or VR-VIII region, where said peptide insertion is surface exposed when the capsid protein is packaged as an AAV particle. The peptide insertion should not delete any residues of the AAV capsid protein. Generally, the peptide insertion occurs in a variable (poorly conserved) region of the capsid protein, compared with other serotypes, and in a surface exposed loop.

A peptide insertion described as inserted “at” a given site refers to insertion immediately after, that is having a peptide bond to the carboxy group of, the residue normally found at that site in the wild type virus. For example, insertion at Q588 in AAV9 means that the peptide insertion appears between Q588 and the consecutive amino acid (A589) in the AAV9 wildtype capsid protein sequence (SEQ ID NO:67). In embodiments, there is no deletion of amino acid residues at or near (within 5, 10, 15 residues or within the structural loop that is the site of the insertion) the point of insertion.

In particular embodiments, the capsid protein is an AAV9 capsid protein (including modified atropic or reduced tropic AAV9 capsids) and the insertion occurs immediately after at least one of the amino acid residues 451 to 461. In particular embodiments, the peptide insertion occurs immediately after amino acid 1451, N452. G453, S454. G455, Q456, N457, Q458, Q459. T460, or L461 of the AAV9 capsid (amino acid sequence SEQ ID NO:67). In certain embodiments, the peptide is inserted between residues S454 and G455 of the AAV9 capsid protein or between the residues corresponding to S454 and G455 of an AAV capsid protein other than an AAV9 capsid protein (amino acid sequence SEQ ID NO:67). In some embodiments, the polypeptide is inserted between residues N452 and G453 of AAV9 capsid protein or between the residues corresponding to N452 and G453 of an AAV capsid protein other than an AAV9 capsid protein (amino acid sequence SEQ ID NO:67). In other embodiments, the polypeptide is inserted before Q458 of the AAV9 capsid protein or before the residue corresponding to Q458 of an AAV capsid protein other than an AAV9 capsid protein (amino acid sequence SEQ ID NO:67).

In other embodiments, provided are engineered capsid proteins comprising targeting peptides heterologous to the capsid protein that are inserted into the AAV capsid protein such that, when incorporated into the AAV vector the heterologous peptide is surface exposed.

In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), serotype rh74 (AAVrh74, versions 1 and 2), serotype rh34 (AAVrh34), serotype hu26 (AAVhu26), serotype rh31 (AAVrh31), serotype hu56 (AAVhu56), serotype hu53 (AAVhu53), serotype rh64R1 (AAVrh64R1), serotype rh46 (AAVrh46), and serotype rh73 (AAVrh73) (see FIG. 8 or Table 7), and the insertion occurs immediately after an amino acid residue corresponding to at least one of the amino acid residues 451 to 461. The alignments of these different AAV serotypes, as shown in FIG. 8, indicates “corresponding” amino acid residues in the different capsid amino acid sequences such that a “corresponding” amino acid residue is lined up at the same position in the alignment as the residue in the reference sequence. In some particular embodiments, the peptide insertion occurs immediately after one of the amino acid residues within: 450-459 of AAV1 capsid (SEQ ID NO:59); 449-458 of AAV2 capsid (SEQ ID NO:60): 449-459 of AAV3 capsid (SEQ ID NO:61): 443-453 of AAV4 capsid (SEQ ID NO:62); 442-445 of AAV5 capsid (SEQ ID NO:63): 450-459 of AAV6 capsid (SEQ ID NO:64): 451-461 of AAV7 capsid (SEQ ID NO:65); 451-461 of AAV8 capsid (SEQ ID NO:66); 451-461 of AAV9 capsid (SEQ ID NO:67); 452-461 of AAV9e capsid (SEQ ID NO:68); 452-461 of AAVrh10 capsid (SEQ ID NO:69); 452-461 of AAVrh20 capsid (SEQ ID NO:70); 452-461 of AAVhu.37 (SEQ ID NO:71); 452-461 of AAVrh74 (SEQ ID NO:72 or SEQ ID NO:80); or 452-461 of AAVrh39 (SEQ ID NO:73), in the sequences depicted in FIG. 8. In certain embodiments, the rAAV capsid protein comprises a peptide insertion immediately after (i.e., C-terminal to) amino acid 588 of AAV9 capsid protein (having the amino acid sequence of SEQ ID NO:67 and see FIG. 8), where said peptide insertion is surface exposed when the capsid protein is packaged as an AAV particle. In other embodiments, the rAAV capsid protein has a peptide insertion that is not immediately after amino acid 588 of AAV9 or corresponding to amino acid 588 of AAV9.

In specific embodiments, the peptide is inserted after 138; 262-272; 450-459; or 585-593 of AAV1 capsid (SEQ ID NO:59); 138; 262-272; 449-458; or 584-592 of AAV2 capsid (SEQ ID NO:60); 138; 262-272; 449-459; or 585-593 of AAV3 capsid (SEQ ID NO:61); 137; 256-262; 443-453; or 583-591 of AAV4 capsid (SEQ ID NO:62); 137; 252-262; 442-445; or 574-582 of AAV5 capsid (SEQ ID NO:63): 138; 262-272; 450-459; 585-593 of AAV6 capsid (SEQ ID NO:64); 138: 263-273; 451-461; 586-594 of AAV7 capsid (SEQ ID NO:65); 138; 263-274; 452-461; 587-595 of AAV8 capsid (SEQ ID NO:66); 138; 262-273; 452-461; 585-593 of AAV9 capsid (SEQ ID NO:67); 138; 262-273: 452-461; 585-593 of AAV9e capsid (SEQ ID NO:68); 138: 263-274: 452-461; 587-595 of AAVrh10 capsid (SEQ ID NO:69); 138; 263-274; 452-461; 587-595 of AAVrh20 capsid (SEQ ID NO:70); 138; 263-274; 452-461; 587-595 of AAVrh74 capsid (SEQ ID NO:72 or SEQ ID NO:80), 138: 263-274; 452-461; 587-595 of AAVhu37 capsid (SEQ ID NO:71); or 138; 263-274; 452-461; 587-595 of AAVrh39 capsid (SEQ ID NO:73) (as numbered in FIG. 8).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to any AAV serotype capsid. Non-limiting examples include a peptide of a human protein in an AAV capsid protein.

In some embodiments, the rAAVs described herein increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution. In some embodiments, the increase in tissue specific cell transduction is at least 2, 10, 20, 30, 40, 50, 60, 70, 80, 90, or 100 fold more than that without the peptide insertion. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type, particularly CNS or heart and/or skeletal muscle in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the peptide insertion. For example, in some embodiments, there is a 50-80 fold increase in genome integration compared to genome integration with the same AAV type without a peptide insert.

In embodiments, the engineered capsids described herein are capsids that comprise a VP1, VP2, and/or VP3 with distinct targeting and/or detargeting mutations). The engineered capsids can be made by a two construct system in which the first construct comprises a nucleotide sequence encoding a cap protein comprising an inactive VP2 initiation site such that VP1 and VP3 proteins are produced and a second construct which comprises a nucleotide sequence encoding a VP2 capsid. Either the first construct of the second construct also encodes the rep protein. Each of the constructs can encode a VP1, VP2 and/or VP3 capsid protein further comprising a targeting and/or detargeting substitution or insertion mutation.

### Flexible Peptide Linkers

In some embodiments, a trans polynucleotide encodes a capsid sequence with a DARPin insert flanked by a pair of flexible peptide linkers. Without being bound by any one theory, such linkers are of adequate length that the DARPin is allowed to take on a tertiary structure (fold) and be at an adequate distance from the capsid to participate in antigen binding. A flexible peptide linker can be composed of flexible residues like glycine and serine so that the adjacent DARPin is free to move relative to the capsid. Commonly used flexible linkers have sequences consisting primarily of stretches of four Gly and one Ser residue (“GS” linker), an example of the most widely used flexible linker having the sequence of (Gly-Gly-Gly-Gly-Ser)n (GGGGS or G4S; SEQ ID NO: 127). By adjusting the copy number “n”, the length of this GS linker can be optimized to achieve appropriate separation of the functional domains, or to maintain necessary inter-domain interactions. Examples include, but are not limited to (Gly-Gly-Gly-Gly-Ser)2 (SEQ ID NO:135). (Gly-Gly-Gly-Gly-Ser)3 (SEQ ID NO: 136), (Gly-Gly-Gly-Gly-Ser)4 (SEQ ID NO: 137), and (Gly-Gly-Gly-Gly-Ser)5 (SEQ ID NO: 138). Besides the GS linkers, many other flexible linkers have been designed for recombinant fusion proteins (Chen, X. et al, Adv Drug Deliv Rev. 2013 Oct. 15; 65(10): 1357-1369). Also provided are PT linkers (Proline-threonine repeating motifs). Also provided are combinations of GS and PT linkers. See, e.g., Table 6.

The construct may be arranged such that the DARPin is at the N-terminus of the capsid protein, followed by one linker and then the capsid protein sequence (for example, N-terminal VP2 fusion).

Alternatively, the construct may be arranged such that a linker is at N-terminus of the DARPin insert, followed by the DARPin and then a second linker at the C-terminus of the DARPin. Capsid protein sequence flanks the linker-DARPin-linker sequence. That is, the components may be arranged as capsid protein portion-linker-DARPin-linker-remaining capsid protein portion.

In embodiments, provided are engineered rAAVs comprising an AAV9 VP1 capsid protein having a 496AAA/NNN498 mutation (SEQ ID NO: 31) and an AAV9 VP2 capsid protein comprising a linker-DARPin-linker motif inserted into between N447 and Q448 of VP2 of AAV9 (VP2 numbering, see SEQ ID NO: 125). Provided are engineered rAAVs comprising an AAV9 VP1 capsid protein having 496AAA/NNN498 substitutions and an VQVGRTS (SEQ ID NO:126) peptide insertion (SEQ ID NO: 122) and an AAV9 VP2 capsid protein comprising a linker-DARPin-linker motif inserted into between N447 and Q448 of VP2 of AAV9 (VP2 numbering, see SEQ ID NO: 125).

Also provided are engineered rAAVs comprising an AAVhu.32 VP1 capsid protein having 496AAA/NNN498 substitutions (SEQ ID NO: 123) and an AAV9 VP2 capsid protein comprising a linker-DARPin-linker motif inserted into between N447 and Q448 of VP2 of AAV9 (VP2 numbering, see SEQ ID NO: 125). Also provided are engineered rAAVs comprising an AAVhu.32 VP1 capsid protein having a 496AAA/NNN498 substitutions and an VQVGRTS (SEQ ID NO:126) peptide insertion (SEQ ID NO: 124) and an AAV9 VP2 capsid protein comprising a linker-DARPin-linker motif inserted into between N447 and Q448 of VP2 of AAV9 (VP2 numbering, see SEQ ID NO: 125).

In embodiments, provided are constructs comprising a nucleotide sequence encoding an AAV9 VP1 protein comprising a 496AAA/NNN498 substitutions (SEQ ID NO: 121). In embodiments, provided are constructs comprising a nucleotide sequence encoding an AAV9 VP1 protein comprising a 496AAA/NNN498 substitutions and a VQVGRTS (SEQ ID NO:126) peptide insertion (SEQ ID NO: 122). In embodiments, provided are constructs comprising a nucleotide sequence encoding an AAVhu32 VP1 protein comprising a 496AAA/NNN498 substitutions (SEQ ID NO: 123). In embodiments, provided are constructs comprising a nucleotide sequence encoding an AAVhu32 VPT protein comprising a 496AAA/NNN498 substitutions and a VQVGRTS (SEQ ID NO: 126) peptide insertion (SEQ ID NO: 123).

In embodiments, provided are constructs comprising a nucleotide sequence encoding an AAV9 VP2 protein comprising an insertion of a DARPin/linker motif in VR4 (VR-IV) or VR8 (VR-VIII). In embodiments, the DARPin/linker motif has a structure of linker-DARPin, DARPin-linker, or linker-DARPin-linker, In embodiments, the linker is a GS linker (glycine-serine linker) or a PT linker (proline-threonine linker). In embodiments, the GS linker is or comprises a (G).S linker, where n=2-10 or is GGS or GGGGS (SEQ ID NO: 127). In embodiments, the linker is or comprises SEQ ID NO: 127. SEQ ID NO: 135, SEQ ID NO: 136, SEQ ID NO: 137, SEQ ID NO: 138, SEQ ID NO: 139 or SEQ ID NO: 140, see, e.g., Table 6.

In embodiments, the DARPin/linker motif is inserted into VR4 (VR-IV). In embodiments, the DARPin/linker motif is inserted within the 452-460 positions of the VP1 protein. In embodiments, amino acids 452-460 of native AAV9 VP1 are deleted in the capsid protein comprising the DARPin. In embodiments, the DARPin/linker motif is inserted into VR8 (VR-VIII). In embodiments, the DARPin/linker motif is inserted within the 585-593 positions of the VP1 protein. In embodiments, amino acids 585-593 of native AAV9 VP1 are deleted in the capsid protein comprising the DARPin. In embodiments, the DARPin/linker motif is inserted between N447 and Q448 of VP2 of AAV9 (VP2 numbering, see SEQ ID NO: 125).

In another aspect, the polypeptide insertion is an antigen binding domain, for example, an scFv, scFv-Fc, single domain antibody, minibody, diabody or other single chain form of an antigen binding domain. Alternatively, the polypeptide insertion is a DARPin as a single or multiple domain binding protein. These targeting or binding domains may be directed to tissue specific cell surface markers, for example, markers, such as cell surface proteins, specific for CNS tissue, muscle tissue, cardiac tissue, peripheral nervous system tissue, etc.

In another aspect, provided are libraries of capsids, including heterologous peptide insertion libraries or libraries of capsids having one or more amino acid substitutions. A heterologous peptide insertion library refers to a collection of rAAV vectors that carry the same peptide insertion at different insertion sites in the virus capsid, e.g., at different positions within a given variable region of the capsid or different variant peptides or even one or more amino acid substitutions. Provided are methods of screening the rAAVs having capsids from the library for enhance of improved properties such as tissue tropism, including enhanced transduction in CNS or cardiac and/or skeletal muscle tissue and, including, reduced transduction in liver and/or DRG cells. Generally, the capsid proteins used comprise AAV genomes that contain modified rep and cap sequences to prevent the replication of the virus under conditions in which it could normally replicate (co-infection of a mammalian cell along with a helper virus such as adenovirus). The members of the peptide insertion libraries may then be assayed for functional display of the peptide on the rAAV surface, tissue targeting and/or gene transduction.

### 5.2.5 Additional AAV Capsid Insertion Sites

The follow summarizes insertion sites for the peptides or polypeptides described herein immediately after amino acid residues of AAV capsid VP1 proteins as set forth below (see also FIG. 8):
AAV1: 138: 262-272; 450-459; 595-593; and in embodiments, between 453-454 (SEQ ID NO:59).
AAV2: 138; 262-272; 449-458; 584-592; and in embodiments, between 452-453 (SEQ ID NO:60).
AAV3: 138; 262-272; 449-459; 585-593; and in embodiments, between 452-453 (SEQ ID NO:61).
AAV4: 137; 256-262; 443-453; 583-591; and in embodiments, between 446-447 (SEQ ID NO:62).
AAV5: 137; 252-262; 442-445; 574-582; and in embodiments, between 445-446 (SEQ ID NO:63).
AAV6: 138; 262-272; 450-459; 585-593; and in embodiments, between 452-453 (SEQ ID NO:64).
AAV7: 138; 263-273; 451-461; 586-594; and in embodiments, between 453-454 (SEQ ID NO:65).
AAV8: 138; 263-274; 451-461; 587-595; and in embodiments, between 453-454 (SEQ ID NO:66).
AAV9: 138; 262-273; 452-461; 585-593; and in embodiments, between 454-455 (SEQ ID NO:67).
AAV9e: 138: 262-273; 452-461; 585-593; and in embodiments, between 454-455 (SEQ ID NO:68).
AAVrh10: 138; 263-274; 452-461; 587-595; and in embodiments, between 454-455 (SEQ ID NO:69).
AAVrh20: 138; 263-274; 452-461; 587-595; and in embodiments, between 454-455 (SEQ ID NO:70).
AAVrh39: 138; 263-274; 452-461; 587-595; and in embodiments, between 454-455 (SEQ ID NO:73).
AAVrh74: 138; 263-274; 452-461; 587-595; and in embodiments, between 454-455 (SEQ ID NO:72 or SEQ ID NO:80).
AAVhu.37: 138; 263-274; 452-461; 587-595; and in embodiments, between 454-455 (SEQ ID NO:71)

In embodiments, the peptide insertion occurs between amino acid residues 588-589 of the AAV9 capsid, or between corresponding residues of another AAV type capsid as determined by an amino acid sequence alignment (for example, as in FIG. 8). In particular embodiments, the peptide insertion occurs immediately after amino acid residue 1451 to L461, S268 and Q588 of the AAV9 capsid sequence, or immediately after corresponding residues of another AAV capsid sequence (FIG. 8).

In some embodiments, one or more peptide insertions can be used in a single system. In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

### 5.2.6 AAV Vectors

Also provided are AAV vectors (or particles) comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV4, AAVS. AAV6, AAV7, AAV8, AAV9, AAV10, AAV1l, AAV12, AAV13, AAV14, AAV15, AAV16, AAV.rh8. AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13. AAV.HSC14, AAV.HSC15, AAV.HSC16, AAVrh34. AAVhu26, AAVrh31, AAVhu56, AAVhu53. AAVrh64R1, AAVrh46, and AAVrh73, or other rAAV particles, or combinations of two or more thereof. In some embodiments, AAV based vectors provided herein comprise components from one or more of AAV1, AAV2, AAV3, AAV4. AAV5, AAV6, AAV7. AAV8, AAV9, AAV10, AAV1l, AAV12, AAV13, AAV14, AAV15, AAV16, AAV.rh8. AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12. AAV.HSC13, AAV.HSC14, AAV.HSC15, or AAV.HSC16, AAVrh34, AAVhu26, AAVrh31, AAVhu56, AAVhu53, AAVrh64R1, AAVrh46, and AAVrh73, or other rAAV particles, or combinations of two or more thereof serotypes. In some embodiments, rAAV particles comprise a capsid protein at least 80% or more identical. e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10. AAV.rh20, AAV.rh39, AAV.Rh74, AAV.RHM4-1, AAV.hu37, AAV.Anc80, rAAV.Anc80L65, AAV.7m8, AAV.PHP.B. AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, AAVrh34, AAVhu26, AAVrh31, AAVhu56. AAVhu53, AAVrh64R1, AAVrh46, and AAVrh73, or a derivative, modification, or pseudotype thereof. These engineered AAV vectors may comprise a genome comprising a transgene encoding a therapeutic protein or nucleic acid.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entiretv). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments. rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments. rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSCS, HSC6, HSC7, HSC8, HSC9, HSC10, HSC11, HSC12, HSC13, HSC14, HSC15, or HSC16, each of which is incorporated by reference in its entirety.

In some embodiments. rAAV particles have a capsid protein disclosed in Intl. Appl. Publ. No. WO 2003/052051 (see, e.g., SEQ ID NO:2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication). WO 2006/068888 (see, e.g., SEQ ID NOs: 1 and 3-6 of '888 publication). WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication). WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18(2):171-82; McCarty et al, 2001, Gene Therapy. 8(16):1248-1254; U.S. Pat. Nos. 6,596,535; 7,125,717; and 7,456,683, each of which is incorporated herein by reference in its entirety).

### 5.2.7 Nucleic Acids, Plasmid Vectors, Bacterial Host Cells, Packaging Cells

Provided are nucleic acids comprising a nucleotide sequence encoding the rAAV capsid protein incorporating the DARPin insertion as disclosed herein, or encoding an amino acid sequence sharing at least 80% identity therewith and retaining biological activity of the rAAV capsid protein, optionally wherein the nucleotide sequence encoding the rAAV capsid protein is operably linked to a promoter and a polyadenylation sequence.

Also provided are plasmid vectors comprising the nucleic acids disclosed herein, wherein the plasmid vectors are replicable in a bacterial cell. Also disclosed are bacterial host cells comprising the plasmid vectors disclosed herein. Also disclosed are packaging cells which expresses the nucleic acids disclosed herein to produce AAV particles comprising the capsid protein encoded by said nucleotide sequence.

### 5.3. Methods of Making rAAV Particles

Another aspect of the present invention involves making rAAV particles having the capsids disclosed herein. In some embodiments, an rAAV particle is made by providing a nucleotide comprising the nucleic acid sequence encoding any of the capsid proteins described herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the one of the capsid proteins described herein, for example, those with sequences in Table 7 or otherwise described herein (see also FIG. 8), while retaining (or substantially retaining) biological function of the capsid protein.

Provided are methods of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP3-DARPin protein, which comprises a VP3 protein having a DARPin inserted within VR-IV or VR-VIII of the VP3 protein; (b) a polynucleotide encoding afunctional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

In embodiments, (a) comprises: (i) a polynucleotide encoding wild-type or parental (i.e., not having the DARPin insert) VP1, VP2 and VP3 proteins and (ii) a polynucleotide encoding VP3-DARPin protein (i.e., having the parental capsid sequence with the DARPin. In embodiments, (a) comprises: (i) a polynucleotide encoding wild-type VP1 and VP2 proteins, wherein the start codon for VP3 is mutated, and (ii) a polynucleotide encoding VP3-DARPin protein.

In embodiments, (i) the polynucleotide encoding wild-type or parental (i.e, having the same amino acid sequence, including any detargeting amino acid substitutions or insertions but not having a DARPin insert) VP1, VP2 and VP3 proteins or the polynucleotide encoding wild-type or parental VP1 and VP2 proteins and (ii) the polynucleotide encoding functional rep gene are operably linked.

In embodiments, prior to the step of culturing the cell comprising one or more polynucleotides, (i) the polynucleotide encoding wild-type or parental VP1, VP2 and VP3 proteins or the polynucleotide encoding wild-type or parental VP1 and VP2 proteins and (ii) the polynucleotide encoding VP3-DARPin protein (in the parental capsid amino acid sequence) were introduced into the cell on separate plasmids. In embodiments, prior to the step of culturing a cell comprising one or more polynucleotides, (i) the polynucleotide encoding wild-type or parental VP1. VP2 and VP3 proteins or the polynucleotide encoding wild-type or parental VP1 and VP2 proteins and (ii) the polynucleotide encoding VP3-DARPin protein were introduced into the cell at a ratio of about 50:50, about 70:30, about 80:20 or about 90:10.

In embodiments, DARPin is inserted into the VP3 protein (and/or VP1 and/or VP2 protein) at VR-IV or elsewhere in the VP1, VP2, and/or VP3 proteins as discussed above. In embodiments, the DARPin is flanked on either one or both ends by a linker. In embodiments, the linker is a GS linker or PT linker as described above.

In embodiments, the polynucleotide encoding VP3-DARPin protein encodes SEQ ID NO: 132 or SEQ ID NO: 133.

Also provided are methods of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP1-DARPin protein, which comprises a VP1 protein having a DARPin inserted within VR-IV or VR-VIII of the VP1 protein; (b) a polynucleotide encoding a functional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

In embodiments, (a) comprises: (i) a polynucleotide encoding wild-type or parental VP1, VP2 and VP3 proteins and (ii) a polynucleotide encoding VP1-DARPin protein. In embodiments, (a) comprises: (i) a polynucleotide encoding wild-type or parental VP2 and VP3 proteins, wherein the start codon for VP1 is mutated so that the VP1 protein is not translated, and (ii) a polynucleotide encoding VP1-DARPin protein.

In embodiments. (i) the polynucleotide encoding wild-type or parental VP1. VP2 and VP3 proteins or the polynucleotide encoding wild-type or parental VP2 and VP3 proteins and (ii) the polynucleotide encoding functional rep gene are operably linked.

In embodiments, prior to the step of culturing the cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein were introduced into the cell on separate plasmids. In embodiments, prior to the step of culturing a cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein are introduced into the cell at a ratio of about 50:50, about 70:30, about 80:20 or about 90:10.

In embodiments, DARPin is inserted into the VP1 protein at VR-IV or elsewhere in the VP1, VP2, and/or VP3 proteins as discussed above. In embodiments, the DARPin is flanked on either one or both ends by a linker. In embodiments, the linker is a GS linker or PT linker as described above.

In embodiments, the polynucleotide encoding VP1-DARPin protein encodes SEQ ID NO: 114 or SEQ ID NO: 115.

Also provided are methods of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises: culturing a cell comprising one or more polynucleotides, wherein the one or more polynucleotides comprise: (a) one or more polynucleotides encoding VP1, VP2 and VP3 proteins; wherein at least one polynucleotide encodes a VP2-DARPin protein, which comprises a VP2 protein having a DARPin inserted within or at the VR—IV or VR-VIII of the VP2 protein; (b) a polynucleotide encoding afunctional rep gene; (c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and (d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

In embodiments. (a) comprises: (i) a polynucleotide encoding wild-type or parental VP1, VP2 and VP3 proteins and (ii) a polynucleotide encoding VP2-DARPin protein. In embodiments, (a) comprises: (i) a polynucleotide encoding wild-type or parental VP1 and VP3 proteins (e.g., the start codon for the VP2 protein is mutated so that the VP2 protein is not translated from that polynucleotide) and (ii) a polynucleotide encoding VP2-DARPin protein.

In embodiments, (i) the polynucleotide encoding wild-type or parental VP1, VP2 and VP3 proteins or the polynucleotide encoding wild-type or parental VP1 and VP3 proteins and (ii) the polynucleotide encoding functional rep gene are operably linked.

In embodiments, prior to the step of culturing the cell comprising one or more polynucleotides. (i) the polynucleotide encoding wild-type or parental VP1. VP2 and VP3 proteins or the polynucleotide encoding wild-type or parental VP1 and VP3 proteins (e.g., the start codon for the VP2 protein is mutated so that the VP2 protein is not translated from that polynucleotide) and (ii) the polynucleotide encoding VP2-DARPin protein were introduced into the cell on separate plasmids. In embodiments, prior to the step of culturing a cell comprising one or more polynucleotides, (i) the polynucleotide encoding wild-type or parental VP1, VP2 and VP3 proteins or the polynucleotide encoding wild-type or parental VP1 and VP3 proteins and (ii) the polynucleotide encoding VP2-DARPin protein are introduced into the cell at a ratio of about 50:50, about 70:30, about 80:20 or about 90:10.

In embodiments, DARPin is inserted into the VP2 protein at VR-IV or elsewhere in the VP1, VP2, and/or VP3 proteins as discussed above. In embodiments, the DARPin is flanked on either one or both ends by a linker. In embodiments, the linker is a GS linker or PT linker as described above.

In embodiments, the polynucleotide encoding VP2-DARPin protein encodes SEQ ID NO: 119.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Accordingly, provided are nucleic acids encoding the modified capsids described herein, including plasmid vectors, specifically AAV Rep-Cap helper plasmids which comprise the nucleotide sequence encoding the modified capsid described herein (including the modified capsids AAV9.G266A.496NNN/AAA498 (SEQ ID NO:50), AAV9.N272A.496NNN/AAA498 (SEQ ID NO:49), AAV9.496NNN/AAA498.W503R (SEQ ID NO:32), and AAV9.496NNN/AAA498.W503A (SEQ ID NO:51) and these modified capsids further modified with a targeting domain insertion). Provided also are bacterial host cells comprising the AAV Rep-Cap helper plasmid and methods of amplifying and producing the AAV Rep-Cap helper plasmid comprising the nucleotide sequence encoding the modified capsid. Further provided are packaging cells (including insect or mammalian cells) which comprise a nucleotide sequence encoding the modified capsid and which packaging cells produce rAAV vectors having a modified capsid as described herein.

In embodiments, the engineered capsids described herein are capsids that comprise a VP1, VP2, and/or VP3 with distinct targeting and/or detargeting mutations including a retargeting DARPin insertion. The engineered capsids can be made by a two construct system in which the first construct comprises a nucleotide sequence encoding a cap protein comprising an inactive VP3 initiation site and a second construct which comprises a nucleotide sequence encoding a VP3 capsid. In embodiments, the first construct comprises a nucleotide sequence encoding a cap protein comprising an inactive VP2 initiation site and a second construct which comprises a nucleotide sequence encoding a VP2 capsid. In embodiments, the first construct comprises a nucleotide sequence encoding a cap protein comprising an inactive VP1 initiation site and a second construct which comprises a nucleotide sequence encoding a VP1 capsid. Each of the constructs can encode a VP1, VP2 and/or VP3 capsid protein further comprising a targeting and/or detargeting substitution or insertion mutation.

In embodiments, provided are constructs comprising a nucleotide sequence encoding an AAV9 VP1 capsid protein having a 496AAA/NNN498 mutation, an AAV9 VP1 capsid protein having a 496AAA/NNN498 mutation and a peptide insert of VQVGRTS (SEQ ID NO:126) inserted between S454 and G454, an AAVhu.32 capsid protein having a 496AAA/NNN498 mutation or an AAVhu.32 capsid protein having a 496AAA/NNN498 mutation and a peptide insert of VQVGRTS (SEQ ID NO:126) inserted between S454 and G454.

In embodiments, provided are constructs comprising a nucleotide sequence encoding an AAV9 VP2 protein comprising an insertion of a DARPin/linker motif in VR4 or VR8. In embodiments, the DARPin/linker motif has a structure of linker-DARPin, DARPin-linker, or linker-DARPin-linker, In embodiments, the linker is a GS linker (glycine-serine linker) or a PT linker (proline-threonine linker). In embodiments, the GS linker is a (G).S linker, where n=2-10 or is GGS or GGGGS (SEQ ID NO: 127). In embodiments, the DARPin/linker motif is inserted into VR4 (VR-IV). In embodiments, the DARPin/linker motif is inserted within the 452-460 positions of the VP1 protein. In embodiments, amino acids 452-460 of native AAV9 VP1 are deleted in the capsid protein comprising the DARPin. In embodiments, the DARPin/linker motif is inserted into VR8 (VR-VIII). In embodiments, the DARPin/linker motif is inserted within the 585-593 positions of the VP1 protein. In embodiments, amino acids 585-593 of native AAV9 VP1 are deleted in the capsid protein comprising the DARPin. In embodiments, the DARPin/linker motif is inserted between N447 and Q448 of VP2 of AAV9 (VP2 numbering, see SEQ ID NO: 125). In embodiments, the DARPin/linker motif is inserted between N447 and Q448 of VP2 of AAV9 (VP2 numbering, see SEQ ID NO: 125).

TABLE 6
Exemplary Linker Sequences

| SEQ | | |
|---|---|---|
| ID | SEQUENCE | |
| NO | NAME | SEQUENCE |
| 127 | 4GS | GGGGS |
| 135 | 4GSx2 | GGGGGGGGS |
| 136 | 4GSx3 | GGGGSGGGGSGGGGS |
| 137 | 4GSx4 | GGGGSGGGGSGGGGSGGGGS |
| 138 | GS-4GSx4-GS | GSGGGGSGGGGSGGGGGGGGSGS |
| 139 | GS-PT linker-GS | PTPTPTTPTPTPTTPTPTPT |
| 140 | GS-PT linker-GS | GSPTPTPTTPTPTPTTPTPTPTGS |

Standard techniques can be used for recombinant DNA, oligonucleotide synts, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See. e.g., Sambrook et al., Molecular Cloning: A Laboratory Manual (2d ed., Cold Spring Harbor Laboratory Press. Cold Spring Harbor. N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the a capsid protein described herein (see Table 7, e.g.), while retaining the biological function of the engineered capsid. In certain embodiments, the encoded engineered capsid has the sequence of an AAV8.BBB.LD capsid (SEQ ID NO:27), an AAV9.BBB.LD capsid (SEQ ID NO:29), an AAV9.496-NNN/AAA-498 capsid (SEQ ID NO:31), AAV9.496-NNN/AAA-498.503R capsid (SEQ ID NO:32), AAV9.W503R capsid (SEQ ID NO:33), AAV9.Q474A capsid (SEQ ID NO:34), AAV9.N272A.496-NNN/AAA-498 capsid (SEQ ID NO:49) or AAV9.G266A.496-NNN/AAA-498 capsid (SEQ ID NO:50), and AAV9.496NNN/AAA498.W503A (SEQ ID NO:51). Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, AAVrh74, AAVrh34, AAVhu26, AAVrh31, AAVhu56, AAVhu53, AAVrh.46, AAVrh.64.R1, AAV.rh.73 vectors, including with the amino acid substitutions and/or peptide insert as described herein and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to the wild type or unengineered sequence for that AAV type and that retains its biological function.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-527, which is incorporated by reference herein in its entirety

The rAAV vector for delivering the transgene to target tissues, cells, or organs, has a tropism for that particular target tissue, cell, or organ. Tissue-specific promoters may also be used. The construct comprising the transgene, within AAV ITR sequences further can include expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015. Mol Cell 59:149-161).

In a specific embodiment, the recombinant AAVs described herein comprise an artificial genome comprising the following components: (1) AAV2 inverted terminal repeats that flank the expression cassette; (2) control elements, which include a) a promoter and, optionally, enhancer elements to promote expression of the transgene in CNS and/or muscle cells, b) optionally an intron sequence, such as a chicken β-actin intron, and c) a polyadenylation sequence, such as an SV40 polyA or rabbit β-globin poly A signal; and (3) transgene providing (e.g., coding for) a nucleic acid or protein product of interest, including a therapeutic nucleic acid or protein.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COSI, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl2 sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays. e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC*), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 1A-1B below provides a list of transgenes that may be used in any of the rAAV vectors described herein, in particular, in the novel insertion sites described herein, to treat or prevent the disease with which the transgene is associated, also listed in Tables 1A-1B. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 1A

| Disease | Transgene |
|---|---|
| MPS I | alpha-L-iduronidase (IDUA) |
| MPS II (Hunter Syndrome) | iduronate-2-sulfatase (IDS) |
| ceroid lipofuscinosis | (CLN1, CLN2, CLN10, CLN13), a |
| (Batten disease) | soluble lysosomal protein |
| | (CLN5), a protein in the |
| | secretory pathway (CLN11), |
| | two cytoplasmic proteins that |
| | also peripherally associate |
| | with membranes (CLN4, CLN14), |
| | and many transmembrane proteins |
| | with different subcellular |
| | locations (CLN3, CLN6, CLN7, |
| | CLN8, CLN12) |
| MPS IIIa (Sanfilippo type A | heparan sulfate sulfatase |
| Syndrome) | (also called N-sulfoglucosamine |
| | sulfohydrolase (SGSH)) |
| MPS IIIB (Sanfilippo type B | N-acetyl-alpha-D-glucosaminidase |
| Syndrome) | (NAGLU) |
| MPS VI (Maroteaux-Lamy | arylsulfatase B |
| Syndrome) | |
| Morquio syndrome (MPS IV) | Beta galactosidase or |
| | galactosamine-6-sulfatase |
| Gaucher disease (type 1, | Glucocerebrosidase, GBA1 |
| II and III) | |
| Parkinson's Disease | Glucocerebrosidase; GBA1 |
| Parkinson's Disease | dopamine decarboxylase |
| Pompe | acid maltase; GAA |
| Metachromatic leukodystrophy | Aryl sulfatase A |
| MPS VII (Sly syndrome) | beta-glucuronidase |
| MPS VIII | glucosamine-6-sulfate sulfatase |
| MPS IX | hyaluronidase |
| Niemann-Pick disease | sphingomyelinase |
| Niemann-Pick disease without | a npc1 gene encoding a |
| sphingomyelinase deficiency | cholesterol metabolizing enzyme |
| Tay-Sachs disease | Alpha subunit of beta-hexosaminidase |
| Sandhoff disease | both alpha and beta subunit of beta- |
| | hexosaminidase |
| Fabry Disease | alpha-galactosidase |
| Fucosidosis | Fucosidase (FUCA1 gene) |
| Alpha-mannosidosis | alpha-mannosidase |
| Beta-mannosidosis | Beta-mannosidase |
| Wolman disease | cholesterol ester hydrolase |
| Parkinson's disease | Neurturin |
| Parkinson's disease | glial derived growth factor (GDGF) |
| Parkinson's disease | tyrosine hydroxylase |
| Parkinson's disease | glutamic acid decarboxylase. |
| No disease listed | fibroblast growth factor-2 (FGF-2) |
| No disease listed | brain derived growth factor (BDGF) |
| No disease listed | neuraminidase deficiency with |
| (Galactosialidosis | betagalactosidase deficiency |
| (Goldberg syndrome)) | |
| Spinal Muscular Atrophy (SMA) | SMN |
| Friedreich's ataxia | Frataxin |
| Amyotrophic lateral | SOD1 |
| sclerosis (ALS) | |
| Glycogen Storage Disease 1a | Glucose-6-phosphatase |
| XLMTM | MTM1 |
| Crigler Najjar | UGT1A1 |
| CPVT | CASQ2 |
| Rett syndrome | MECP2 |
| Achromatopsia | CNGB3, CNGA3, GNAT2, PDE6C |
| Choroidermia | CDM |
| Danon Disease | LAMP2 |

TABLE 1B

| Disease | Transgene |
|---|---|
| Cystic Fibrosis | CFTR |
| Duchenne Muscular Dystrophy | Mini-Dystrophin or |
| | Micro-dystrophin Gene |
| Limb Girdle Muscular Dystrophy | human-alpha-sarcoglycan |
| Type 2C\|Gamma-sarcoglycanopathy | |
| Advanced Heart Failure | SERCA2a |
| Rheumatoid Arthritis | TNFR:Fc Fusion Gene |
| Leber Congenital Amaurosis | GAA |
| Limb Girdle Muscular Dystrophy | gamma-sarcoglycan |
| Type 2C\|Gamma-sarcoglycanopathy | |
| Retinitis Pigmentosa | hMERTK |
| Age-Related Macular Degeneration | sFLT01 |
| Becker Muscular Dystrophy and | huFollistatin344 |
| Sporadic Inclusion Body Myositis | |
| Parkinson's Disease | GDNF |
| Metachromatic Leukodystrophy | cuARSA |
| (MLD) | |
| Hepatitis C | anti-HCV shRNA |
| Limb Girdle Muscular Dystrophy | hSGCA |
| Type 2D | |
| Human Immunodeficiency Virus | PG9DP |
| Infections; HIV Infections (HIV-1) | |
| Acute Intermittant Porphyria | PBGD |
| Leber's Hereditary Optical | P1ND4v2 |
| Neuropathy | |
| Alpha-1 Antitrypsin Deficiency | alpha1AT |
| Pompe Disease | hGAA |
| X-linked Retinoschisis | RS1 |
| Choroideremia | hCHM |
| Giant Axonal Neuropathy | JeT-GAN |
| Duchenne Muscular Dystrophy | rmicro-Dystrophin |
| X-linked Retinoschisis | hRS1 |
| Squamous Cell Head and Neck | hAQP1 |
| Cancer; Radiation Induced | |
| Xerostomia | |
| Hemophilia B | Factor IX |
| Homozygous FH | hLDLR |
| Dysferlinopathies | rAAVrh74.MHCK7.DYSF.DV |
| Hemophilia B | AAV6 ZFP nuclease |
| MPS I | AAV6 ZFP nuclease |
| Rheumatoid Arthritis | NF-kB.IFN-β |
| Batten/CLN6 | CLN6 |
| Sanfilippo Disease Type A | hSGSH |
| Osteoarthritis | 5IL-1Ra |
| Achromatopsia | CNGA3 |
| Achromatopsia | CNGB3 |
| Ornithine Transcarbamylase (OTC) | OTC |
| Deficiency | |
| Hemophilia A | Factor VIII |
| Mucopolysaccharidosis II | ZFP nuclease |
| Hemophilia A | ZFP nuclease |
| Wet AMD | anti-VEGF |
| X-Linked Retinitis Pigmentosa | PGR |
| Mucopolysaccharidosis Type VI | hARSB |
| Leber Hereditary Optic | ND4 |
| Neuropathy | |
| X-Linked Myotubular Myopathy | MTM1 |
| Crigler-Najjar Syndrome | UGT1A1 |
| Achromatopsia | CNGB3 |
| Retinitis Pigmentosa | hPDE6B |
| X-Linked Retinitis Pigmentosa | RPGR |
| Mucopolysaccharidosis Type 3 B | hNAGLU |
| Duchenne Muscular Dystrophy | GALGT2 |
| Arthritis, Rheumatoid; Arthritis, | TNFR:Fc Fusion Gene |
| Psoriatic; Ankylosing Spondylitis | |
| Idiopathic Parkinson's Disease | Neurturin |
| Alzheimer's Disease | NGF |
| Human Immunodeficiency Virus | tgAAC09 |
| Infections; HIV Infections (HIV-1) | |
| Familial Lipoprotein Lipase | LPL |
| Deficiency | |
| Idiopathic Parkinson's Disease | Neurturin |
| Alpha-1 Antitrypsin Deficiency | hAAT |
| Leber Congenital Amaurosis (LCA) 2 | hRPE65v2 |
| Batten Disease; Late Infantile | CLN2 |
| Neuronal Lipofuscinosis | |
| Parkinson's Disease | GAD |
| Sanfilippo Disease Type | N-sulfoglucosamine sulfohydrolase |
| A/Mucopolysaccharidosis Type IIIA | (SGSH) gene |
| Congestive Heart Failure | SERC2a |
| Becker Muscular Dystrophy and | rAAV1.CMV.huFollistatin344 |
| Sporadic Inclusion Body Myositis | |
| Parkinson's Disease | hAADC-2 |
| Choroideremia | REP1 |
| CEA Specific AAV-DC-CTL | CEA |
| Treatment in Stage IV | |
| Gastric Cancer | |
| Gastric Cancer | MUC1-peptide-DC-CTL |
| Leber's Hereditary Optical | scAAV2-P1ND4v2 |
| Neuropathy | |
| Aromatic Amino Acid Decarboxylase | hAADC |
| Deficiency | |
| Hemophilia B | Factor IX |
| Parkinson's Disease | AADC |
| Leber Hereditary Optic Neuropathy | Genetic: GS010\|Drug: Placebo |
| SMA - Spinal Muscular | SMN |
| Atrophy\|Gene Therapy | |
| Hemophilia A | B-Domain Deleted Factor VIII |
| MPS I | IDUA |
| MPS II | IDS |
| CLN3-Related Neuronal Ceroid- | CLN3 |
| Lipofuscinosis (Batten) | |
| Limb-Girdle Muscular Dystrophy, | hSGCB |
| Type 2E | |
| Alzheimer Disease | APOE2 |
| Retinitis Pigmentosa | hMERKTK |
| Retinitis Pigmentosa | RLBP1 |
| Wet AMD | Anti-VEGF antibody |

For example, a rAAV vector comprising a transgene encoding glial derived growth factor (GDGF) finds use treating/preventing/managing Parkinson's disease. Generally, the rAAV vector is administered systemically. For example, the rAAV vector may be provided by intravenous, intrathecal, intra-nasal, and/or intra-peritoneal administration.

In certain embodiments, the transgene encodes a microdystrophin (for example, as disclosed in WO2021/108755, WO2002/029056, WO2016/115543, WO2015/197232, WO2016/177911, U.S. Pat. No. 7,892,824B2. U.S. Pat. No. 9,624,282B2, and WO2017221145, which are hereby incorporated by reference in their entireties) and is useful for treatment of dystrophinopathies, such as muscular dystrophy. rAAV particles having a serotype of AAV7, AAV8, AAV9, AAVrh.10, AAVrh.46, AAVrh.64.R1, and AAVrh.73, or an engineered forms thereof, may be useful for delivery of transgenes encoding microdystrophins or other dystrophinopathy therapeutic proteins to muscle cells, including skeletal and/or cardiac muscle, while having reduced delivery to liver cells, for treatment of muscular dystrophies, such as, Duchenne Muscular Dystrophy.

In particular aspects, the rAAVs of the present invention find use in delivery to target tissues, or target cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject an rAAV where the peptide insertion is a homing peptide. In the case of Parkinson's, for example, a rAAV vector comprising a peptide insertion that directs the rAAV to neural tissue can be used, in particular, where the peptide insertion facilitates the rAAV in crossing the blood brain barrier to the CNS.

For a disease or disorder associated with neural tissue, an rAAV vector can be used that comprises a peptide insertion from a neural tissue-homing domain, such as any described herein. Diseases/disorders associated with neural tissue include Alzheimer's disease, amyotrophic lateral sclerosis (ALS), amyotrophic lateral sclerosis (ALS), Battens disease, Batten's Juvenile NCL form, Canavan disease, chronic pain, Friedreich's ataxia, glioblastoma multiforme, Huntington's disease, Late Infantile neuronal ceroid lipofuscinosis (LINCL), lysosomal storage disorders, Leber's congenital amaurosis, multiple sclerosis. Parkinson's disease, Pompe disease, Rett syndrome, spinal cord injury, spinal muscular atrophy (SMA), stroke, and traumatic brain injury. The vector further can contain a transgene for therapeutic/prophylactic benefit to a subject suffering from, or at risk of developing, the disease or disorder (see Tables 1A-1B).

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10¹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention (gene therapy vectors) is typically a single treatment.

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered, in combination with the gene therapy vector, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In particular embodiments, such as where the transgene is intended to be expressed in the CNS, the vector is administered via lumbar puncture or via cisterna magna.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotherapy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In certain embodiments, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

### Example 1

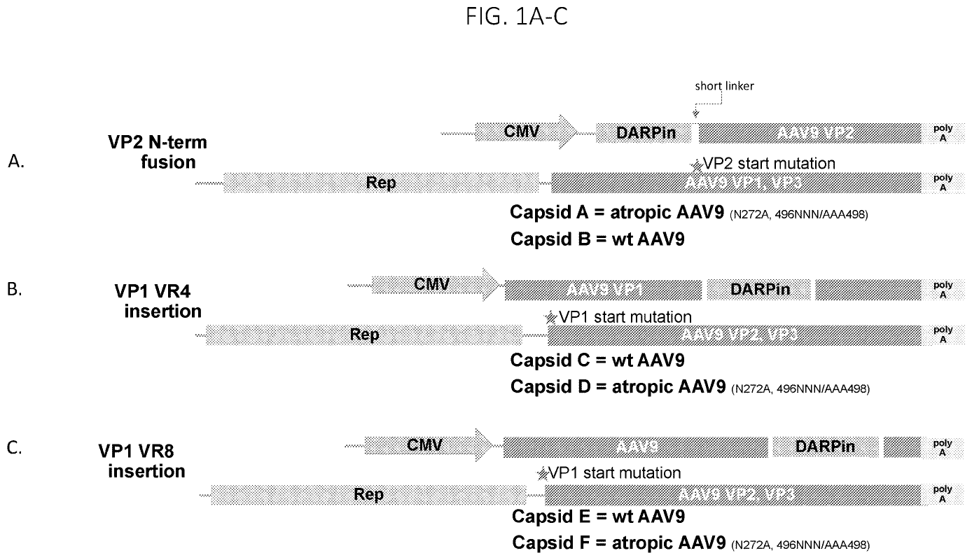

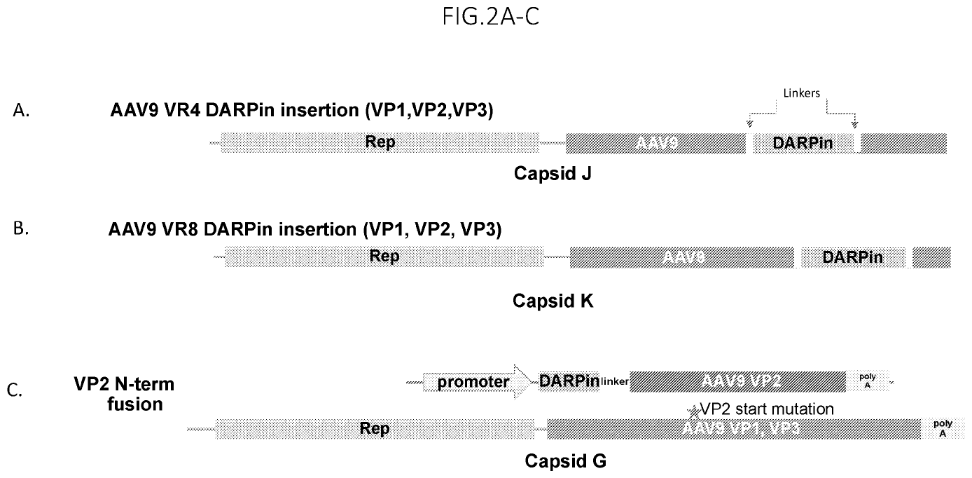

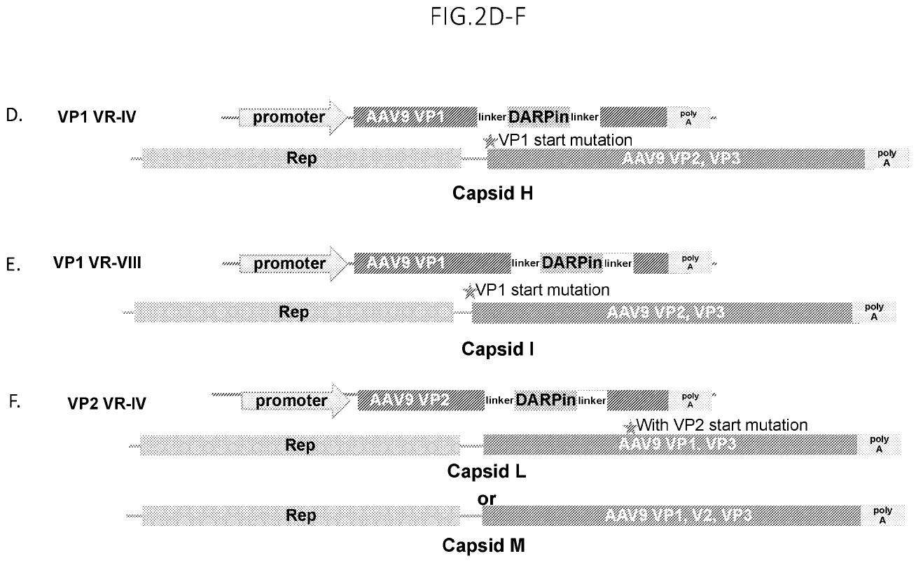

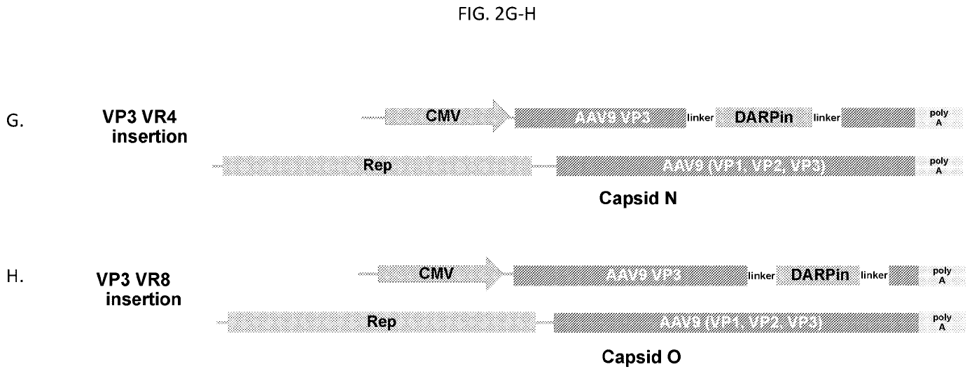

A. The following plasmids were made to understand engineered capsids that contain DARPin insertions, their expression and the engineered capsid's ability to package into AAV vectors. DARPin sequences were inserted at different locations in AAV9 VP1 and VP2 proteins (or a modified “atropic” AAV9) and expressed from a separate plasmid construct by a constitutive promoter. For each capsid, the native VP1 or VP2 protein was knocked-out by mutating its initiation codon (see FIGS. 1A-C and FIGS. 2A-F):

### Capsid A: AtropicAAV9-DARPin-GGS-VP2 Capsid (“VP2-NTerm”)

trans (rep/cap) plasmid “atropic” AAV9 (N272A.NNN) having a mutated VP2 start codon (will express only VP1 and VP3)
secondary plasmid encoding wtAAV9 VP2 having the DARPin-GGS fused to the N-Terminus

### Capsid B: WtAAV9-DARPin-GGS-VP2 Capsid (“Atropic VP2-NTerm”)

trans (rep/cap) plasmid wtAAV9 having a mutated VP2 start codon (will express only VP1 and VP3)
secondary plasmid encoding wtAAV9 VP2 having DARPin-GGS fused to the N-Terminus

### Capsid C: WtAAV9-GGGGS-DARPin-GGGGA Capsid (“VP1.VR4”)

trans (rep/cap) plasmid wtAAV9 having a mutated VP1 start codon (expresses VP2 and VP3)
secondary plasmid encoding wtAAV9 having a GGGGS-DARPin-GGGGA-460 inserted within the 452-460 positions of the VP1 protein (deleted 452-460 of native AAV9 VP1)

### Capsid D: AtropicAAV9-GGGGS-DARPin-GGGGA Capsid (“VP1.VR4”)

trans (rep/cap) plasmid atropicAAV9 having a mutated VP1 start codon (expresses VP2 and VP3)
secondary plasmid encoding atropicAAV9 having a GGGGS-DARPin-GGGGA-460 inserted within the 452-460 positions of the VP1 protein (deleted 452-460 of native AAV9 VP1)

### Capsid E: WtAAV9-GGGGS-DARPin-GGGGA Capsid (“VP1.VR8”)

trans (rep/cap) plasmid wtAAV9 having a mutated VP1 start codon (will express only VP1 and VP3) (expresses VP2 and VP3)
secondary plasmid encoding wtAAV9 having a GGGGS-DARPin-GGGGA-460 inserted within the 585-593 positions of the VP1 protein (deleted 585-593 of native AAV9 VP1)

### Capsid F: AtropicAAV9-GGGGS-DARPin-GGGGA Capsid (“VP1.VR8”)

trans (rep/cap) plasmid atropicAAV9 having a mutated VP1 start codon (will express only VP1 and VP3) (expresses VP2 and VP3)
secondary plasmid encoding atropicAAV9 having a GGGGS-DARPin-GGGGA-460 inserted within the 585-593 positions of the VP1 protein (deleted 585-593 of native AAV9 VP1)

B. In some experiments, an example DARPin was used as the targeting ligand that binds to the glutamate receptor subunit GluA4 (2K19). GluA4 is expressed in Parvalbumin (PV+) intemeurons (GABAergic interneurons) of the brain, with low abundance observed in striatum, higher abundance observed in cortex, and higher abundance observed in thalamic reticular nucleus.

Additional plasmids were made:

### Capsid G: “PT-Linker” VP2-NTerm

Trans and secondary plasmid was made analogously to capsid B, however using PT linkers instead of GS linkers

### Capsid H: “PT-Lnker” VP1-VR4

Trans and secondary plasmid was made analogously to capsid C, however using PT linkers instead of GS linkers

### Capsid I: “PT-Linker” VP1-VR8

Trans and secondary plasmid was made analogously to capsid E, however using PT linkers instead of GS linkers

### Capsid J: AAV9 VR4-DARPin Insertion (FIG. 2A)—Trans Only

### Capsid K: AAV9 VR8 DARPn Insertion (FIG. 2B)—Trans Only

### Capsid L: VP2-VR4

Trans Rep/AAV9 Cap having the VP2 start codon mutated (see capsid B)
Secondary=VP2 only with DARPin inserted at VR-IV (amino acid residue 454) flanked by linkers

C. In other experiments, example DARPin (Athebody®) binding proteins were used as the targeting ligand fused to AAV that binds to a human target receptor (hTR). Additional plasmids to enable the production of these AAV-DARPin fusions were constructed as follows:

### Capsid M: VP2-VR4 (FIG. 2F)

Trans Rep/AAV9 Cap expressing VP1, VP2 and VP3 (“wildtype”)
Secondary=VP2 only with DARPin inserted at VR-IV and flanked by linkers (e.g. inserted after amino acid residue N452 or S454; G453 and S454 may be considered part of a GS linker; any or all of amino acids G453, S454, G455, Q456, N457, or Q458 may be deleted)

### Capsid N: VP3-VR4 (FIG. 2G)

Trans Rep/AAV9 Cap expressing VP1, VP2 and VP3 (“wildtype”)
Secondary=VP3 only with DARPin inserted at VR-IV and flanked by linkers (e.g. inserted after amino acid residue N452 or S454; G453 and S454 may be considered part of a GS linker; any or all of amino acids G453, S454, G455, Q456, N457, or Q458 may be deleted)

### Capsid O: VP3-VR8 (FIG. 2H)

Trans Rep/AAV9 Cap expressing VP1, VP2, VP3
Secondary=VP3 only with DARPin inserted at VR-VIII (inserted within amino acid residues 585-593) flanked by linkers

Each VP2-only or VP3-only polynucleotide (plasmid) is under the control of a strong promoter, e.g. CMV or CAG or other suitable promoter. The expression cassette encoding VP2 or VP3 is operably linked to a 3′-polyadenylation signal (polyA). In certain examples where the linker is a GS linker and the insertion site is at VR-IV then amino acids G453 and S454 may be considered part of the GS linker. In any event, the VR-IV DARPIN-linker fusion is immediately before Q459 of AAV9. In the VR4 insertion example, G455, Q456, N457, or Q458 are deleted upon insertion of the DARPin-linker fusion. Capsids M, N or O are manufactured with a wildtype cap trans gene for AAV9, however alternatively a cap trans gene having the VP2 or VP3 start codon mutated may be utilized in the production.

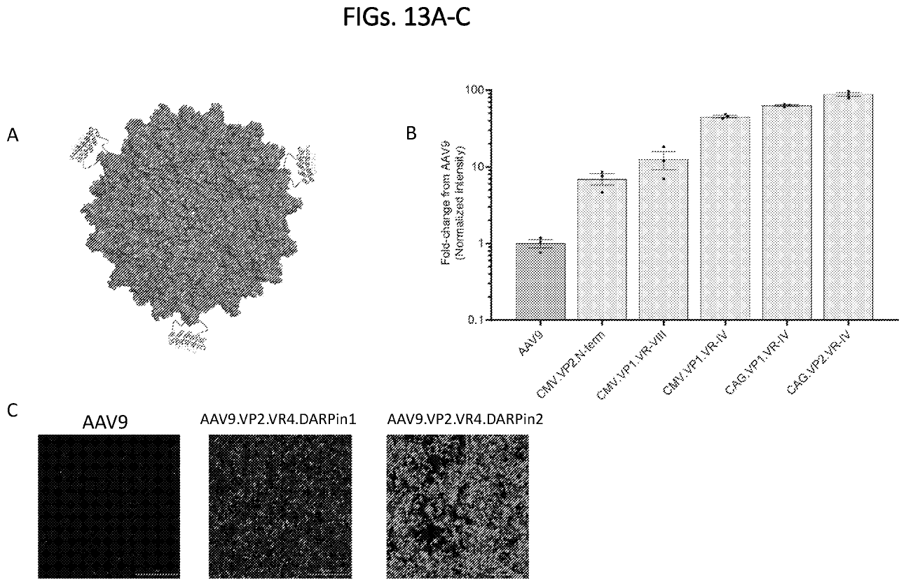

Vector production having capsid-DARPin insertions at or within VR-IV consistently produced vectors with greater transduction capability (see, for example, FIG. 13B).

### Example 2: AAV-DARPin Characterization

### Production and Titer Measurement

Briefly, vector was made and harvested by transfecting the packaging plasmids (trans rep/cap and secondary plasmids as noted above, cis plasmid carrying the genome/transgene, and helper gene plasmid) in 3.05E6 viable cells in 10 mL of media in 250 mL. Following a 3-day culture the vectors were harvested by pelleting then conducting freeze/thaw lysing. Vector was treated with DNAse and proteinase K to collect DNA. ddPCR was conducted using FAM channel and GFP primer set to measure the titer from each batch. rAAV vectors are produced at quantities similar to or better than parent vector.

TABLE 2

| Vector | GC/mL |
|---|---|
| wt AAV9 | 7.58E+11 |
| atropic AAV9 | 6.99E+11 |
| wt AAV9 with DARPin-VP2 | 1.02E+12 |
| atropic AAV9 with DARPin-VP2 | 2.72E+11 |
| wt AAV9 with DARPin VP1.VR4 | 9.25E+11 |
| wt AAV9 with DARPin VP1.VR8 | 4.77E+11 |
| atropic AAV9 with DARPin VP1.VR4 | 2.05E+11 |
| atropic AAV9 with DARPin VP1.VR8 | 4.65E+11 |

### In Vitro Transduction

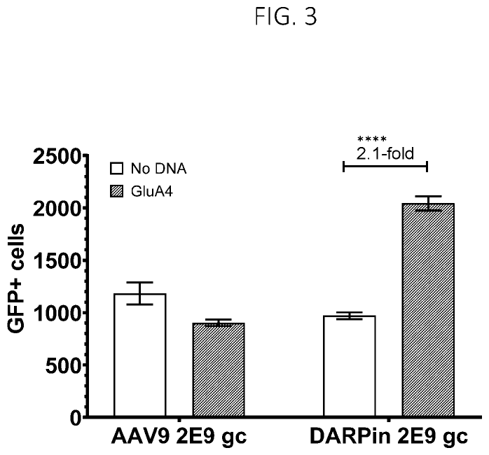

In vitro transduction of HEK293 cells over-expressing rat GluA4 receptor showed that wtAAV9-DARPin (GluA4-targeting DARPin in DARPin capsid B with DARPin fused to the VP2 protein of AAV9) could increase expression of the transgene by 2.1-fold. See Table 3 and FIG. 3.

TABLE 3

| | | | | | GFP+ GluA4- | | | |
|---|---|---|---|---|---|---|---|---|
| | GFP+ Untransfected | | | | transfected cells | | | |
| | Cells (No DNA) | | | | (GluA4) | | | |
| wLAAV9 | 1225 | 872 | 1308 | 1327 | 820 | 959 | 891 | 940 |
| 2E9 gc | | | | | | | | |
| Capsid B | 1024 | 1009 | 970 | 880 | 1906 | 2054 | 1987 | 2222 |
| (GluA4- | | | | | | | | |
| DARPin) | | | | | | | | |
| 2E9 gc | | | | | | | | |

### In Vivo Distribution

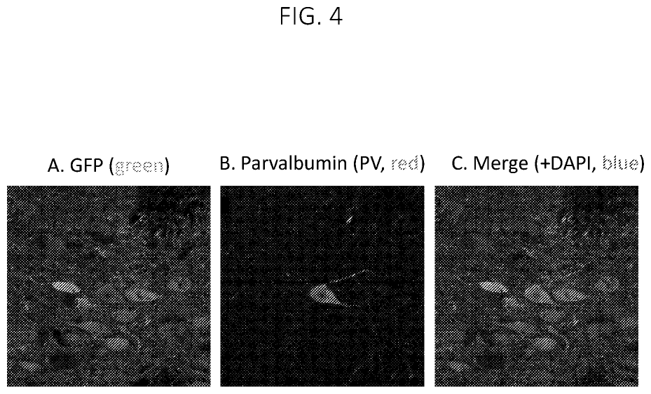

The vectors were also injected into mouse brain by intra-striatal injection and the AAV9 vector displaying a GluA4-targeted DARPin is active in vivo as shown by robust transgene (GFP) expression in mouse brain using fluorescent imaging techniques. PV+ cells could be detected as DAPI positive (stained) cells and overlaid (merged) with GFP images (FIG. 4).

### Cell-Based Assays

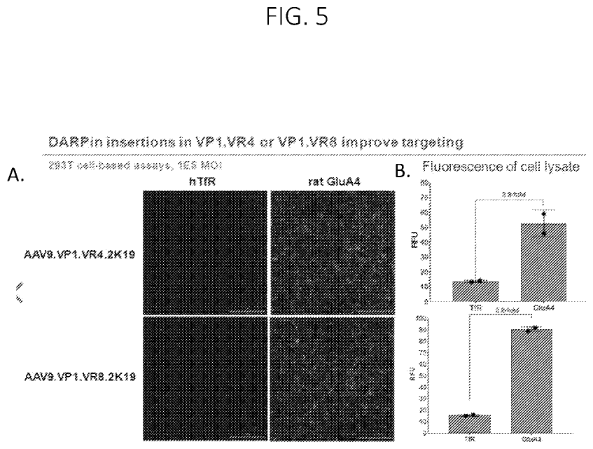

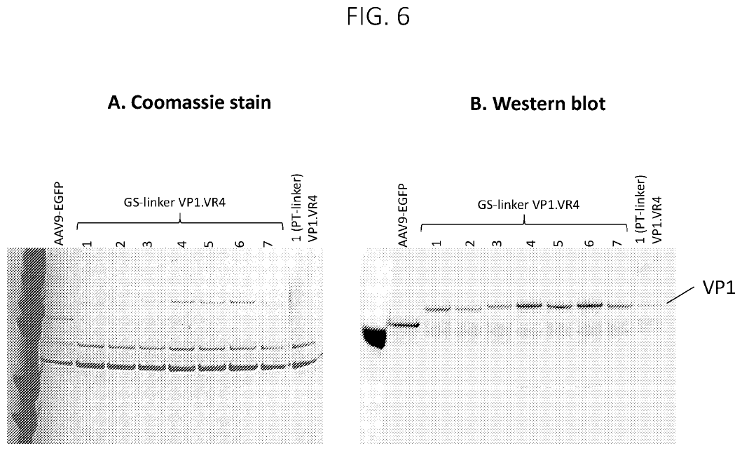

Capsid C (VP1.VR4) and Capsid E (VP1.VR8) vectors were produced by an analogous technique to that described above and tested in HEK293 cell-based assays. In vitro activity was measured by transduction of HEK293-HumTargetReceptor and HEK293-AAVR cells at 1E5 MOI (FIG. 5A) followed by quantification of TdTomato expression (FIG. 5B). Capsid C improved expression of a fluorescent marker transgene by 3.9 fold, and Capsid E improved expression of transgene by 5.8-fold compared to wtAAV9 and target receptor in HEK293 cells (FIG. 5B).

### Titers and VP Ratios

DARPin libraries made by known methods, for example, made with two (N2C) or three (N3C) ankyrin repeats and were constructed by diversifying 15 or 20 amino acid positions, respectively, with initial library sizes of >1E12. Four rounds of selection against a biotinylated extracellular domain of a Human Target Receptor were performed. After selection, top binders were sequenced and evaluated by competition studies, and for their binding kinetics, stability, etc.

Various Capsid C (VP1.VR4) DARPin AAV vectors were made with GFP transgene (only the DARPin inserted in each capsid were different) and tested for titer and VP ratio by Western Blot (FIG. 6). AAV-DARPin preps were produced at 50 mL scale. Vectors were produced by four plasmid transfection of a suspension HEK293-derived cell line packaging a CAG.eGFP or CAG.TdTomato genome cassette. Small-scale production for screening was performed at 50 mL scale and vectors were purified from cell lysates by AAV9-resin batch-binding and elution. Larger-scale production was performed at 1 L scale and vectors were purified by PEGprecipitated ultra-centrifugation. Vector titration was performed by ddPCR following DNaseI/proteinase K digest.

VP ratios were characterized by SDS-PAGE Coomassie stain, western blot with anti-VP1 (Progen 61056) or anti-VP1,2,3 (Progen 61058-488) antibodies, and by PerkinElmer Labchip.

DARPin-AAV titers and VP ratios were adequate for all variants with different DARPins, and comparable to wildtype AAV9 for some variants, as shown in Table 4 and FIG. 6.

TABLE 4

| vector | GC/ml | GC load (ddPCR) |
|---|---|---|
| AAV9.CAG.GFP | 4.43E+13 | 6.67E+10 |
| DARPin 1- Capsid C | 4.50E+13 | 6.67E+10 |
| (VP1.VR4) | | |
| DARPin 2- Capsid C | 5.40E+13 | 6.67E+10 |
| (VP1.VR4) | | |
| DARPin 3- Capsid C | 5.00E+13 | 6.67E+10 |
| (VP1.VR4) | | |
| DARPin 4- Capsid C | 2.80E+13 | 6.67E+10 |
| (VP1.VR4) | | |
| DARPin 5- Capsid C | 6.40E+13 | 6.67E+10 |
| (VP1.VR4) | | |
| DARPin 6- Capsid C | 2.10E+13 | 6.67E+10 |
| (VP1.VR4) | | |
| DARPin 7- Capsid C | 4.00E+13 | 6.67E+10 |
| (VP1.VR4) | | |
| DARPin 8- Capsid C | 3.95E+11 | 6.42E+09 |
| (VP1.VR4) | | |

Capsid C (VP1.VR4) with DARPin 1 insertion (Epitope A) exhibits a higher titer than the same DARPin inserted in Capsid E (VP1.VR8). GS linkers were comparable to PT linkers as shown by Capsid C (GS-VP1.VR4) or PT linker Capsid G (PT-VP1.VR4) parameters. See Table 5 and FIG. 6.

TABLE 5

| | | | | ddPCR | | | |
|---|---|---|---|---|---|---|---|
| | | Insertion | | titer | GC | Expected | Expected |
| Construct | DARPin | Site | Linker | (GC/mL) | loaded | VP1 size | VP2 size |
| 1 | 1 | VP1.VR4 | PT-24 | 3.95E+11 | 6.42E+09 | 101.8 | 66.3 |
| 2 | 1 | VP1.VR8 | PT-24 | 1.68E+11 | 2.73E+09 | 101.8 | 66.3 |
| 3 | 1 | Nterm. VP2 | PT-24 | 4.87E+11 | 7.92E+09 | 81.4 | 85.1 |
| 4 | 1 | VP1.VR4 | GS-24 | 3.68E+11 | 5.98E+09 | 100.4 | 66.3 |
| 5 | 1 | VP1.VR8 | GS-24 | 1.95E+11 | 3.18E+09 | 100.4 | 66.3 |
| 6 | 1 | Nterm. VP2 | GS-24 | 3.68E+11 | 5.98E+09 | 81.4 | 84.4 |
| 7 | — | — | — | 5.42E+11 | 8.81E+09 | 81.4 | 66.3 |
| (AAV9) | | | | | | | |
| 8 | 2K19 | VP1.VR8 | GGGGS | 2.55E+13 | 4.14E+10 | 91.0 | 66.3 |
| 9 | 2K19 | Nterm.VP2 | GGS | 5.60E+13 | 1.82E+10 | 81.4 | 76.2 |

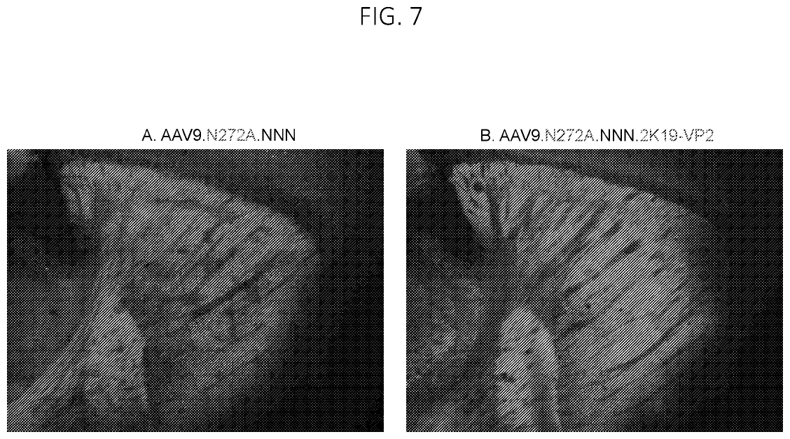

AAV9 Vectors having the capsid amino acid mutations N272A and 496-NNN/AAA-498 substitutions (N272A.NNN), also called atropic vectors, remain highly active when administered directly into the mouse brain (intra-striatum) and the GluA4-DARPin vector having the AAV9-atropic mutations increases overall transduction in the brain (FIG. 7). Cell specificity will be measured. Analogous experiments, however administering vectors by other routes including intravenously, will be conducted.

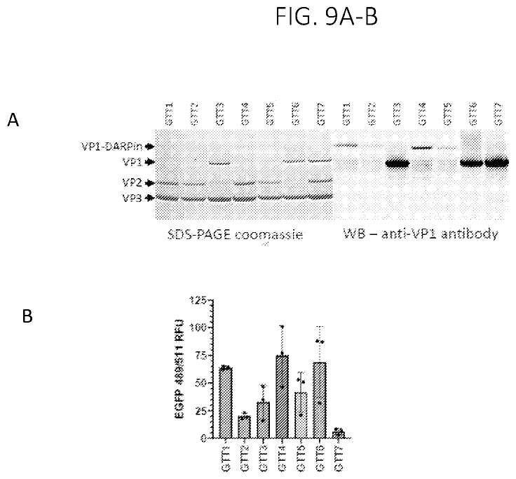

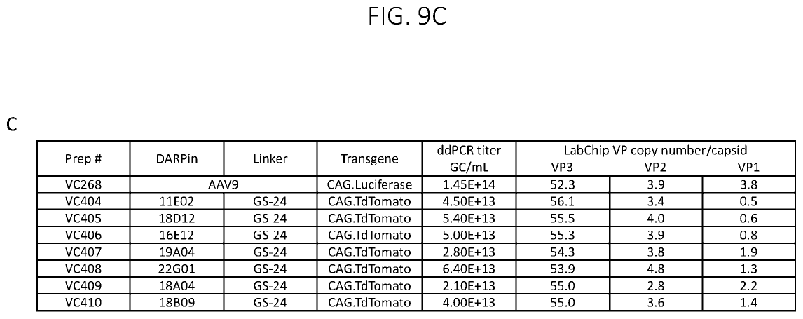

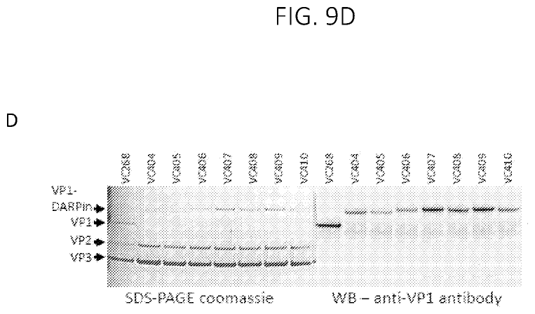

VR-IV appears to be an optimal site for DARPin incorporation and transduction. eGFP fluorescence was measured 48 hrs post-transduction of HEK293-hTargetReceptor (hTR) cells at 1E5 MOI. (FIG. 9A-B) and up to 2.2 VP1-Darpin copies per capsid on average were detected (FIG. 9C). One liter (1 L) scale production with purification and ultracentrifugation also yielded stable capsids with adequate VP ratios as measured by SDS page and Western (FIG. 9D).

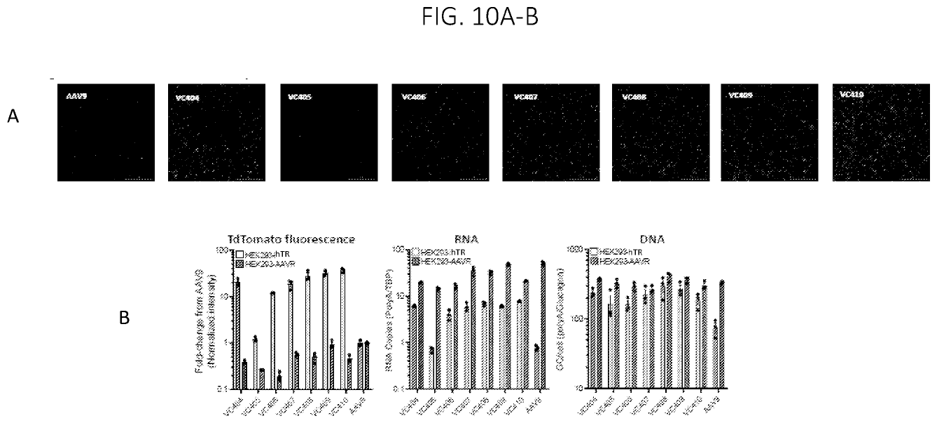

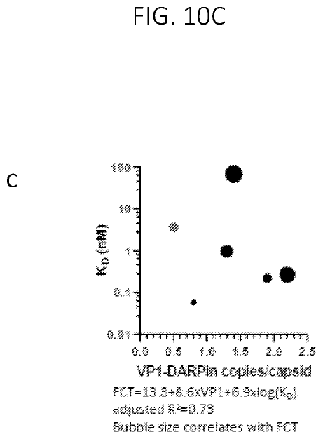

The DARPins inserted in AAV9 mediated up to 38-fold increased transduction of HEK293-hTargetReceptor (hTR) cells versus AAV9. TdTomato fluorescence, RNA copies and GC/cell were measured 48 hours post-transduction of HEK293-hTR and HEK293-AAVR cells at 1E5 MOI with DARPin-AAV vectors. Multiple regression analysis with two independent variables identified a positive correlation between fold-change transduction (FCT) of HEK293-hTR cells, VP1-DARPin copies and DARPin KD (i.e. increased FC-trans, decreased VP-DARPin copies, decreased binding affinity). FIGS. 10A-C.

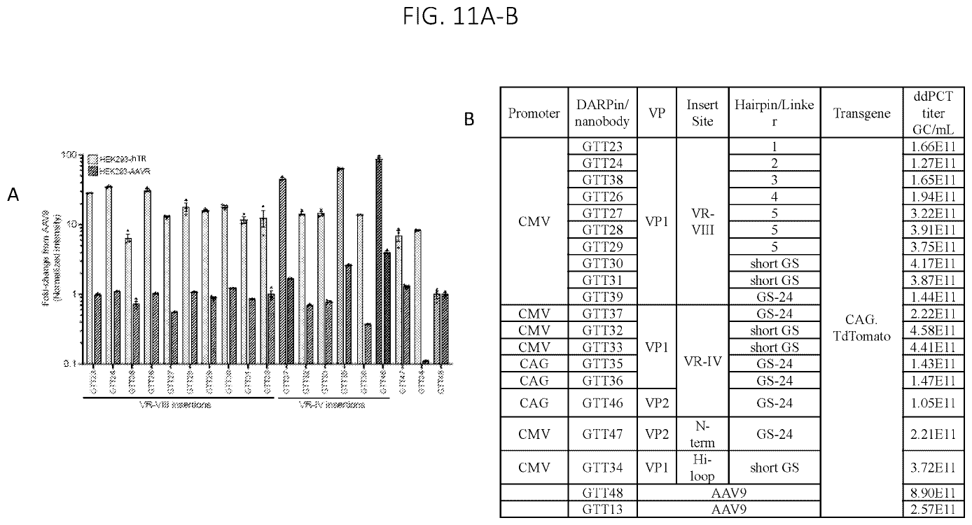

It was observed that the CAG promoter and DARPin insertion in VP2 VR-IV mediated a 90-fold increased transduction versus AAV9 where DARPins targeting a human TR induced greater transduction in the HEK293-hTR cells (FIG. 11A). Various linkers were utilized, as well as promoters, in the construction of DARPin-AAVs. Some stabilizing linkers in VR-VIII were found to increase transduction activity versus AAV9 more than a GS-24 linker (30-fold vs 10-fold). The stronger CAG promoter was found to increase VP-DARPin incorporation and transduction activity more than the CMV promoter (60-fold vs 45-fold).

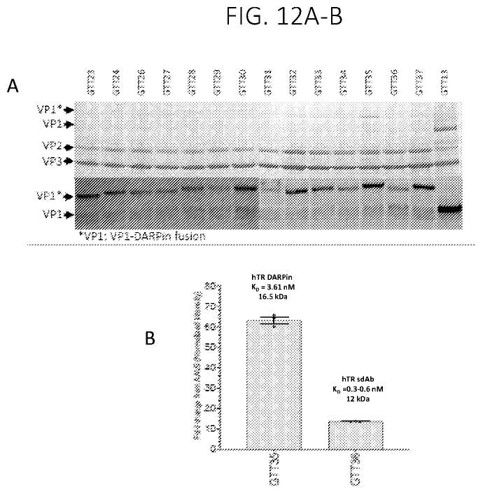

The VR-IV insertion in VP2 with the CAG promoter increased transduction more than in VP1 (90-fold vs 60-fold) (e.g. GTT35, FIGS. 11A-B). However, circularly permuting the DARPin to bring the N- and C-termini next to each other or other insertion sites had minimal or no beneficial effect on transduction. SDS-PAGE and anti-VP1 Western Blots of AAV-DARPin constructs were performed for the 50 mL scale AAV-DARPin productions (FIG. 12A). DARPin incorporation and transduction of HEK293-hTR cells was superior to an anti-TR single-domain antibody (FIG. 12B). The observations consistently show that VR4 insertions and longer linkers lead to improved transduction through DARPin target binding. In some DARPin vectors. AAVR-expressing cell assays also showed that AAV transduction was comparable to wtAAV9 or increased compared to wtAAV9 without the aid of target receptor binding, possibly due to stabilization (or lack of interference) of the AAV-AAVR receptor interaction in these VR4-DARPin vectors.

### Example 3: Combining Multiple Capsid Engineering Approaches to Develop AAV Vectors with Novel Properties

As the field of gene therapy has grown, there has been an increased need for a range of new vectors with novel profiles. From high specificity for single target tissues or organs, to wide tropism but with reduced toxicity, engineered AAVs could allow for gene therapy to be applied in many disease indications. Hundreds of naturally occurring AAVs have been described, and countless more exist as AAV persists in primates, other mammals, and avian species. Almost all vectors currently in clinical use are derived from nature. However, none are specific for any cell or tissue type.

Adeno associated virus (AAV) tissue tropism is a property determined by the protein capsid. AAV capsids are assemblies of 60 VP proteins. VP1, 2, and 3 are produced from a single gene, cap, and differ only on the N-terminus. With such a large and complex structure there are multiple avenues for discovering, identifying, and engineering AAV capsids with desirable traits. For regions of the capsid surface where protein interactions are understood, rational engineering in the form of amino acid substitution can be undertaken to modify vector properties or larger sections of sequence swapped between capsids. More commonly, directed evolution is used as an unbiased approach to enhance capsids with new properties. Additionally, proteins (or other ligands) of known function can be fused or coupled to the capsid surface to add novel properties to the vector. While many new AAV capsids have been described, it remains unclear how modular any of the modifications might be and which combinations could lead to further improvement.

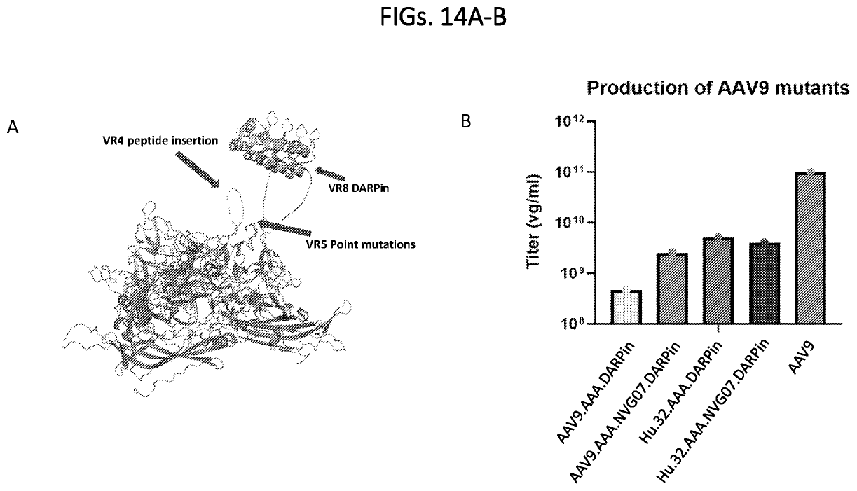

Starting with an AAV9 vector, specific residues were mutated in the three-fold spike region on the surface to introduce liver detargeting (FIG. 14A). Multiple peptide insertion libraries into different surface exposed loops were built into the liver detargeted vector (AAV.AAA) and selected for capsids which continued to show low transduction of liver relative to AAV9, but which recovered the ability to transduce other organs of interest in NHPs. Three rounds of selection were performed in NHPs with barcoded capsid libraries to select top peptide insertions, followed by in vitro, in vivo, and structural analysis of the capsids. Using this approach, NAVIGATE (Novel AAV Vector Intelligent Guided Adaptation Through Evolution) directed evolution platform, multiple novel capsids were identified which retained liver detargeting but showed enhanced muscle tropism. A mutant AAV9.AAA capsid was identified, having the NVG07 peptide inserted at VR4, which retained low liver tropism and also showed an improved muscle as well as CNS tropism profile (Intl. Appl. Publ. No. WO2024/044725; and Mercer, A. et al. “Combining multiple capsid engineering approaches to develop AAV vectors with novel properties” European Society of Gene and Cell Therapy 2023 Congress, Oct. 24-27, 2023, Brussels, BE).

AAV9 capsids were further modified to insert Designed Ankyrin Repeat Proteins (DARPins) selected to bind a human target receptor as a model for protein insertions on the capsid surface (FIG. 13A).

Athebody® DARPins are small “plug & play” binding proteins composed of ankyrin repeat units: an N- and C-terminal cap along with one to three internal repeats. Multiple parameters of the DARPin-AAV fusion construct were optimized and there was a 90-fold increase in transduction of cells over-expressing the DARPin target receptor as compared to an unmodified AAV9 vector, particularly for insertions at VR-IV (FIG. 13B). Also, as second generation anti-TR DARPins having stronger binding affinity to the hTR than first generation DARPin selections exhibited high transduction of hTR-expressing cells (FIG. 13C).

Additionally, AAV9, mutant AAV9 or AAV.hu32 capsids were further modified. A DARPin was incorporated into AAV9.AAA. AAV.hu32.AAA, AAV9.AAA.NVG07 and Hu32.AAA.NVG07 capsids. Note that the AAV9 or hu32 capsid was modified to have mutations (NNN→AAA) at VR5, the DARPin binding protein was inserted at VR8 and the “NVG07” peptide was inserted at VR4 in these examples of highly engineered capsids (FIG. 14B). Packaged vectors with the engineered capsids were detected and displayed two or more DARPin binding proteins per capsid (AAV particle) on average (FIG. 13A), although production of vectors with engineered capsids were detected at lower titers than wildtype AAV9 vector (FIG. 14B).

### Example 4: Manufacturing of Engineered Capsids

Optimal titer of produced vectors having sufficient binding capabilities afforded by a capsid-DARPin fusion can be difficult to achieve. As such, another approach was undertaken to achieve robust manufacturing without sacrificing efficacy. Capsid M (VP2.VR4) and Capsid N (VP3.VR4) vectors were produced by four plasmid transfection technique similar to that described above and tested in HEK293 cell-based assays. By inserting the DARPin in the VP2 or VP3 gene encoded by a separate plasmid, the amount of trans and secondary trans plasmids could be adjusted based on the ratio of DNA needed in a transfection mixture for AAV vector production.

AAV-DARPin preps were produced at 50 mL or 1 L scale. Vectors were produced by four plasmid transfection of a suspension HEK293-derived cell line packaging a CAG.eGFP or CAG.TdTomato genome cassette. Quadruple transfection comprises a mixture of four plasmids with transfection reagent (e.g. PEI) as follows: 1) a rep/cap (trans)-expressing plasmid, needed to form the VPs of the capsid not containing the DARPin insert, 2) a secondary trans plasmid, which plasmid also encodes for a DARPin inserted in a capsid protein, 3) a cis plasmid carrying a genome (such as a therapeutic or fluorescent marker transgene), and 4) sufficient helper genes to allow for formation of an rAAV particle. In a common triple transfection, the ratio of DNA mass or concentration of each of the trans:cis:helper is typically in the range 2:1:1 to 1:1:1 to 1:2:1 to 1:1:2. In a quadruple transfection, the portion of trans DNA typically added to the transfection is further split to accommodate two trans plasmids. Small-scale production for screening was performed at 50 mL scale and vectors were purified from cell lysates by AAV9-resin batch-binding and elution. Larger-scale production was performed at 1 L scale and vectors were purified by PEGprecipitated ultra-centrifugation. Vector titration was performed by ddPCR following DNaseI/proteinase K digest.

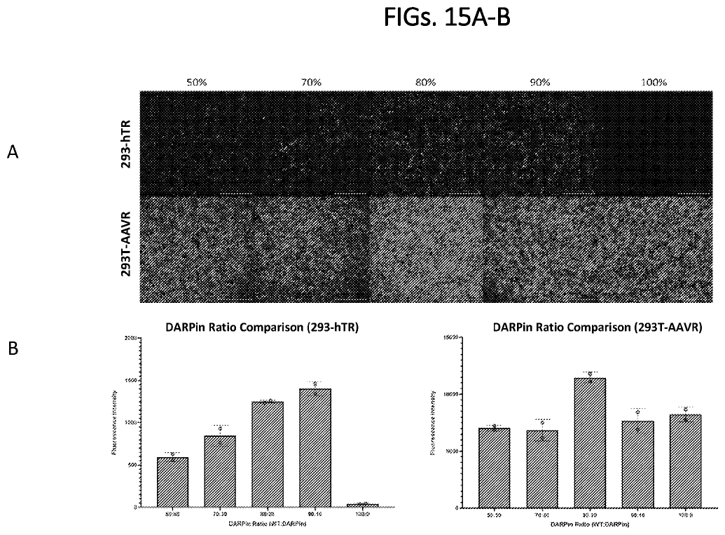

In vitro activity of several first generation DARPin-AAV fusions (DARPins binding to Epitope A of hTR) were measured by transduction of HEK293-HumTargetReceptor (hTR) and HEK293T-AAVR cells at 1E5 MOI (FIG. 15A) followed by quantification of TdTomato expression (measured in relative fluorescent units; RFUs) (FIG. 15B). It was observed that 20 or 10% DARPin (secondary) plasmid (80:20 or 90:10 ratio of wt trans and secondary trans plasmids together) would provide the best transduction efficiency for AAV-DARPin binding to Epitope A.

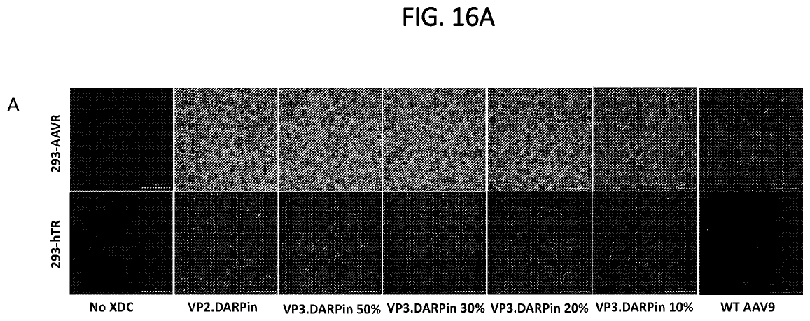

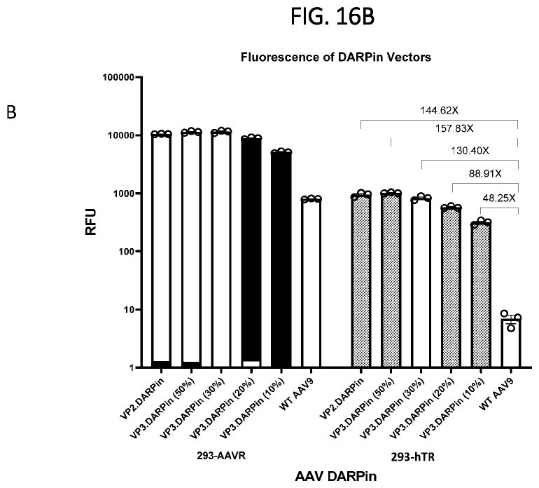

In vitro activity of several second generation DARPin-AAV fusions (DARPins bind to Epitope B) were also measured by transduction of HEK293-HumTargetReceptor (hTR) and HEK293-AAVR cells at 1E5 MOI (FIG. 16A) followed by quantification of TdTomato expression (RFUs) (FIG. 16B). AAV-DARPin (VR4 insertion) binding to Epitope B yielded vectors with high transduction efficiency, particularly at a 50:50 or 70:30 ratio trans:secondary trans plasmids utilizing VP3-VR4 plasmids.

### 6.21. Capsid Sequences

Table 7 provides the amino acid sequences of certain engineered capsid proteins described and/or may be used in studies described herein. Heterologous peptides and amino acid substitutions are indicated in gray shading.

Table 8 provides nucleotide sequences coding for certain capsids that may require more than one plasmid construct during transfection. Amino acid sequences of capsids made from one trans plasmid are also provided.

Table 9 provides the amino acid sequences of capsids.

Table 10 provides the amino acid sequences of liver detargeting or atropic capsid inserts.

TABLE 7
Capsid Amino Acid Sequences

| | Insert or | | |
|---|---|---|---|
| Capsid | Sub- | | |
| Name | stitution | Amino Acid Sequence | |
| PHP.S | QAVRT | 1 | [symbol] |
| (California | SL (SEQ | 61 | [symbol] |
| Institute | ID | 121 | [symbol] |
| of | NO: 16) | 181 | [symbol] |
| Technology- | (588_589) | 241 | [symbol] |
| Chan | | 301 | [symbol] |
| et al | | 361 | [symbol] |
| 2017) | | 421 | [symbol] |
| | | 481 | [symbol] |
| | | 541 | [symbol] |
| | | 601 | [symbol] |
| | | 661 | [symbol] |
| | | 721 | [symbol] |
| PHP.SH | QAVRT | 1 | [symbol] |
| | SH | 61 | [symbol] |
| | (SEQ ID | 121 | [symbol] |
| | NO: 17) | 181 | [symbol] |
| | (588_589) | 241 | [symbol] |
| | | 301 | [symbol] |
| | | 361 | [symbol] |
| | | 421 | [symbol] |
| | | 481 | [symbol] |
| | | 541 | [symbol] |
| | | 601 | [symbol] |
| | | 661 | [symbol] |
| | | 721 | [symbol] |
| PHP.B | TLAVPF | 1 | [symbol] |
| (California | K (SEQ | 61 | [symbol] |
| Institute | ID | 121 | [symbol] |
| of | NO: 20) | 181 | [symbol] |
| Technology | (588_589) | 241 | [symbol] |
| GenBank | | 301 | [symbol] |
| entry: | | 361 | [symbol] |
| ALU85156.1- | | 421 | [symbol] |
| Dever | | 481 | [symbol] |
| man et | | 541 | [symbol] |
| al | | 601 | [symbol] |
| 2016) | | 661 | [symbol] |
| | | 721 | [symbol] |
| PHP.eB | TLAVPF | 1 | [symbol] |
| (California | K (SEQ | 61 | [symbol] |
| Institute | ID | 121 | [symbol] |
| of | NO: 20) | 181 | [symbol] |
| Technology- | and | 241 | [symbol] |
| Chan | (588_589) | 301 | [symbol] |
| et al | | 361 | [symbol] |
| 2017) | | 421 | [symbol] |
| | | 481 | [symbol] |
| | | 541 | [symbol] |
| | | 601 | [symbol] |
| | | 661 | [symbol] |
| | | 721 | [symbol] |

| AAV8.BBB | A269S | [symbol] | 60 |
|---|---|---|---|
| | | [symbol] | 120 |
| | | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV8. | A269S, | [symbol] | 60 |
| BBB. | 498_NNN/ | [symbol] | 120 |
| LD | AAA_500 | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | S263G/ | [symbol] | 60 |
| BBB | S269T/ | [symbol] | 120 |
| | A273T | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | S263G/ | [symbol] | 60 |
| BBB. | S269T/ | [symbol] | 120 |
| LD | A273T, | [symbol] | 180 |
| | 496_NNN/ | [symbol] | 240 |
| | AAA_498 | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAVrh10. | 498_NNN/ | [symbol] | 50 |
| LD | AAA_500 | [symbol] | 100 |
| | | [symbol] | 150 |
| | | [symbol] | 200 |
| | | [symbol] | 250 |
| | | [symbol] | 300 |
| | | [symbol] | 350 |
| | | [symbol] | 400 |
| | | [symbol] | 450 |
| | | [symbol] | 500 |
| | | [symbol] | 550 |
| | | [symbol] | 600 |
| | | [symbol] | 650 |
| | | [symbol] | 700 |
| | | [symbol] | |
| AAV9. | 498_NNN/ | [symbol] | 60 |
| 496NNN/ | AAA_500 | [symbol] | 120 |
| AAA498 | | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | 496NNN/ | [symbol] | 60 |
| 496NNN/ | AAA498, | [symbol] | 120 |
| AAA498. | W503R | [symbol] | 180 |
| W503R | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | W503R | [symbol] | 60 |
| W503R | | [symbol] | 120 |
| | | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | Q474A | [symbol] | 60 |
| Q474A | | [symbol] | 120 |
| | | [symbol] | 180 |
| | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | Bone1, | [symbol] | 60 |
| S454- | DDDDD | [symbol] | 120 |
| D8 | DDD | [symbol] | 180 |
| | (SEQ ID | [symbol] | 240 |
| | NO: 2) | [symbol] | 300 |
| | (454_455) | [symbol] | 360 |
| | | [symbol] | 420 |
| | | [symbol] | 481 |
| | | [symbol] | 541 |
| | | [symbol] | 601 |
| | | [symbol] | 661 |
| | | [symbol] | 721 |
| | | [symbol] | |
| AAV9 | Brain1, | [symbol] | 60 |
| S454- | LSSRLD | [symbol] | 120 |
| Brain1 | A (SEQ | [symbol] | 180 |
| | ID NO: | [symbol] | 240 |
| | 3) | [symbol] | 300 |
| | (454_455) | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | Brain2/ | [symbol] | 60 |
| S454- | Brain 1C, | [symbol] | 120 |
| Brain2 | CLSSRL | [symbol] | 180 |
| | DAC | [symbol] | 240 |
| | (SEQ ID | [symbol] | 300 |
| | NO: 4) | [symbol] | 360 |
| | (454_455) | [symbol] | 420 |
| | | [symbol] | 482 |
| | | [symbol] | 542 |
| | | [symbol] | 602 |
| | | [symbol] | 662 |
| | | [symbol] | 722 |
| | | [symbol] | |
| AAV9 | Kidney 1, | [symbol] | 60 |
| S454- | LPVAS | [symbol] | 120 |
| Kidney | (SEQ ID | [symbol] | 180 |
| 1 | NO: 6) | [symbol] | 240 |
| | (454_455) | [symbol] | 300 |
| | | [symbol] | 360 |
| | | [symbol] | 420 |
| | | [symbol] | 478 |
| | | [symbol] | 538 |
| | | [symbol] | 598 |
| | | [symbol] | 658 |
| | | [symbol] | 718 |
| | | [symbol] | |
| AAV9 | Kidney 2/ | [symbol] | 60 |
| S454- | Kidney 1 | [symbol] | 120 |
| Kidney | C, | [symbol] | 180 |
| 2 | CLPVAS | [symbol] | 240 |
| | C (SEQ | [symbol] | 300 |
| | ID NO: | [symbol] | 360 |
| | 5) | [symbol] | 420 |
| | (454_455) | [symbol] | 480 |
| | | [symbol] | 540 |
| | | [symbol] | 600 |
| | | [symbol] | 660 |
| | | [symbol] | 720 |
| | | [symbol] | |
| AAV9 | Muscle1, | [symbol] | 60 |
| S454- | ASSLNI | [symbol] | 120 |
| Muscle | A (SEQ | [symbol] | 180 |
| 1 | ID NO: | [symbol] | 240 |
| | 7) | [symbol] | 300 |
| | (454_455) | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | Tfr1, | [symbol] | 60 |
| S454- | HAIYPR | [symbol] | 120 |
| Tfr1 | H (SEQ | [symbol] | 180 |
| | ID NO: | [symbol] | 240 |
| | 10) | [symbol] | 300 |
| | (454_455) | [symbol] | 360 |
| | | [symbol] | 420 |
| | | [symbol] | 480 |
| | | [symbol] | 540 |
| | | [symbol] | 660 |
| | | [symbol] | 660 |
| | | [symbol] | 720 |
| | | [symbol] | |
| AAV9 | Tfr3, | [symbol] | 60 |
| S454- | RTIGPS | [symbol] | 120 |
| Tfr3 | V (SEQ | [symbol] | 180 |
| | ID NO: | [symbol] | 240 |
| | 12) | [symbol] | 300 |
| | (454_455) | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9 | Tfr4, | [symbol] | 60 |
| S454- | CRTIGP | [symbol] | 120 |
| Tfr4 | SVC | [symbol] | 180 |
| (AAV9 | (SEQ ID | [symbol] | 240 |
| S454- | NO: 13) | [symbol] | 300 |
| TfR3C) | (454_455) | [symbol] | 360 |
| | | [symbol] | 420 |
| | | [symbol] | 482 |
| | | [symbol] | 542 |
| | | [symbol] | 602 |
| | | [symbol] | 662 |
| | | [symbol] | 722 |
| | | [symbol] | |
| AAV9. | SITLVK | [symbol] | 60 |
| 588Ad | STQTV | [symbol] | 120 |
| (9 588 | (SEQ ID | [symbol] | 180 |
| Ad) | NO: 14), | [symbol] | 240 |
| | DLC- | [symbol] | 300 |
| | AS1 | [symbol] | 360 |
| | (588_589) | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | TILSRS | [symbol] | 60 |
| 588 | TQTG | [symbol] | 120 |
| Herp | (SEQ ID | [symbol] | 180 |
| (9 588 | NO: 15), | [symbol] | 240 |
| Hep) | DLC- | [symbol] | 300 |
| | AS2, | [symbol] | 360 |
| | 588_589 | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |

| | | | |
|---|---|---|---|
| AAVP | SITLVK | 1 | [symbol] |
| HPeB. | STQTV | 61 | [symbol] |
| VP2Ad | (SEQ ID | 121 | [symbol] |
| | NO: 14), | | [symbol] |
| | DLC- | 191 | [symbol] |
| | AS1 | 251 | [symbol] |
| | (138_139) | 311 | [symbol] |
| | | 371 | [symbol] |
| | | 431 | [symbol] |
| | | 491 | [symbol] |
| | | 551 | [symbol] |
| | | 611 | [symbol] |
| | | 671 | [symbol] |
| | | 731 | [symbol] |
| AAVP | TILSRS | 1 | [symbol] |
| HPeB. | TQTG | 61 | [symbol] |
| VP2Herp | (SEQ ID | 121 | [symbol] |
| | NO: 15), | | [symbol] |
| | DLC- | 192 | [symbol] |
| | AS2, | 252 | [symbol] |
| | (138_139) | 312 | [symbol] |
| | | 372 | [symbol] |
| | | 432 | [symbol] |
| | | 492 | [symbol] |
| | | 552 | [symbol] |
| | | 612 | [symbol] |
| | | 672 | [symbol] |
| | | 732 | [symbol] |

| Rh.64R1 | R697W | [symbol] | |
|---|---|---|---|
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| AAV9. | N272A | [symbol] | 60 |
| N272A. | 498_NNN/ | [symbol] | 120 |
| 496NNN/ | AAA_500 | [symbol] | 180 |
| AAA498 | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | G266A | [symbol] | 60 |
| G266A | 498_NNN/ | [symbol] | 120 |
| 496NNN/ | AAA_500 | [symbol] | 180 |
| AAA498 | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |
| AAV9. | | [symbol] | 60 |
| 496NNN/ | | [symbol] | 120 |
| AAA498. | | [symbol] | 180 |
| W503A | | [symbol] | 240 |
| | | [symbol] | 300 |
| | | [symbol] | 360 |
| | | [symbol] | 420 |
| | | [symbol] | 480 |
| | | [symbol] | 540 |
| | | [symbol] | 600 |
| | | [symbol] | 660 |
| | | [symbol] | 720 |
| | | [symbol] | |

| | | |
|---|---|---|
| rh34 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.31 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| rh.31 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.12 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.13 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.21 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.26 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.53 | — | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| hu.56 | - | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| Rh.46 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |

| SEQ | Brief | |
|---|---|---|
| ID NO | Description | Sequence |
| 113 | Capsid | *GSPTPTPTTPTPTPTTPTPTPTGSAPGKKRPVEQSPQEPDSSAGIGKSGAQPAKKRLNFGQTGDTESV |
| | G | PDPQPIGEPPAAPSGVGSLTMASGGGAPVADNNEGADGVGSSSGNWHCDSQWLGDRVITTSTRTWALPT |
| | PT- | YNNHLYKQISNSTSGGSSNDNAYFGYSTPWGYFDFNRFHCHFSPRDWQRLINNNWGFRPKRLNFKLFNI |
| | linker | CVKEVTDNNGVKTIANNLTSTVQVFTDSDYQLPYVIGSAHEGCLPPFPADVEMIPQYGYLTLNDGSQAV |
| | VP2 | GRSSFYCLEYFPSQMLRTGNNFQFSYEFENVPFHSSYAHSQSLDRLMNPLIDQYLYYLSKTINGSGQNQ |
| | [*DARP | CTIKFSVAGPSNMAVQGRNYTPGPSYRQQRVSTTVTQNNNSEFAWPGASSWAINGRNSLMNPGPAMASH |
| | in | KEGEDRFFPLSGSLIFGKQGTGRDNVDADKVMITNEEEIKTTNPVATESYGQVATNHQSAQACAQTGWV |
| | inserted | CNQGILPGMVWQDRDVYLQGPIWAKIPHTDGNFHPSPLMGGFGMKHPPPQILIKNTPVPADPPTAFNKD |
| | 5′ of the | KLNSFITQYSTGQVSVEIEWELQKENSKRWNPEIQYTSNYYKSNNVEFAVNTEGVYSEPRPIGTRYLTR |
| | linker] | NL |
| 114 | Capsid | MAADGYLPDWLEDNLSEGIREWWALKPGAPQPKANQQHQDNARGLVLPGYKYLGPGNGLDKGEPVNAAD |
| | H | AAALEHDKAYDQQLKAGDNPYLKYNHADAEFCERLKEDTSFGGNLGRAVFQAKKRLLEPLGLVEEAAKT |
| | PT- | APGKKRPVEQSPQEPDSSAGIGKSGAQPAKARLNFGQTGDTESVPDPQPIGEPPAAPSGVGSLTMASGG |
| | linker | GAPVADNNEGADGVGSSSGNWHCDSQWLGDRVITTSTRTWALPTYNNHLYKQISNSTSGGSSNDNAYFG |
| | VP1.VR4 | YSTPWGYFDFNRFHCHFSPRDWQRLINNNWGFRPKRLNFKLFNIQVKEVTINNGVKTIANNLTSTVQVE |
| | [*DARP | TDSDYQLPYVLGSAHEGCLPPFPADVFMIPQYGYLTLNDGSQAVGRSSFYCLEYIPSQMLRTGNNFQFS |
| | in | YEFENVPFHSSYAHSQSLDRLMNPLIDQYLYYLSKTINGSPTPTPTTPTPTPTTPTPTPIGS*GSPTPT |
| | inserted | PTTPTPTPTTPTPTPTGSQTLKFSVAGPSNMAVQGRNYIPGPSYRQQRVSTTVTQNNNSEFAWPGASSW |
| | after | ALNGRNSLMNPGPAMASHKEGEDRFFPLSGSLIFGKQGTGRDNVDADKVMITNEEEIKTINPVATESYG |
| | serine | QVATNHQSAQAQAQTGWVQNQGILPCMVWQDRDVYLQGPIWAKIPHTDGNFHPSPLMGGFGMKHPPPQI |
| | 476] | LIKNTPVPADPPTAFNKDKINSFITQYSTGQVSVEIEWELQKENSKRWNPEIQYTSNYYKSNNVEFAVN |
| | | TEGVYSEPRPIGTRYLTRNL |
| 115 | Capsid I | MAADGYLPDWLEDNLSEGIREWWALKPGAPQPKANQQHQDNARGLVLPGYKYLGPGNGLDKGEPVNAAD |
| | PT- | AAALEHDKAYDQQLKAGDNPYLKYNHADAEFCERLKEDTSFGGNLGRAVFQAKKRLLEPLGLVEEAAKT |
| | linker | APGKKRPVEQSPQEPDSSAGIGKSGAQPAKKRLNFGQTGDTESVPDPQPIGEPPAAPSGVGSLTMASGG |
| | VP1.VR8 | GAPVADNNEGADGVGSSSGNWHCDSQWLGDRVITTSTRTWALPTYNNHLYKQISNSTSGGSSNDNAYFG |
| | [*DARP | YSTPWGYFDFNRFHCHFSPRDWQRLINNNWGFRPKRLNFKLFNIQVKEVTINNGVKTIANNLTSTVQVF |
| | in | TDSDYQLPYVLGSAHEGCLPPFPADVFMIPQYGYLTLNDGSQAVGRSSFYCLEYFPSQMLRTGNNFQFS |
| | inserted | YEFENVPFHSSYAHSQSLDRLMNPLIDQYLYYLSKTINGSGQNQQTLKFSVAGPSNMAVQGRNYIPGPS |
| | after | YRQQRVSTTVTQNNNSEFAWPGASSWALNGRNSLMNPGPAMASHKEGEDRFFPLSGSLIFGKCGTGRDN |
| | serine | VDADKVMITNEEEIKTTNPVATESYGQVATNHQSAGSPTPTPTTPTPTPTTPTPTPTGS* |
| | 611] | GSPTPTPTTPTPTPTTPTPTPTGSTGWVQNQGILPGMVWQDRDVYLQGPIWAKIPHTDGNFHPSPLMGG |
| | | FGMKHPPPQILIKNTPVPADPPTAFNKDKLNSFITQYSIGQVSVEIEWELQKENSKRWNPEIQYTSNYY |
| | | KSNNVEFAVNTEGVYSEPRPIGTRYLTRNL |
| 117 | AAV9 | MAPGKKRPVEQSPQEPDSSAGIGKSGAQPAKKRLNFGQTGDTESVPDPQPIGEPPAAPSGVGSLTMASG |
| | VP2 | GGAPVADNNEGADGVGSSSGNWHCDSQWLGDRVITTSTRTWALPTYNNHLYKQISNSTSGGSSNDNAYF |
| | only | GYSTPWGYFDFNRFHCHFSPRDWQRLINNNWGFRPKRLNFKLENIQVKEVTDNNGVKTIANNLTSTVQV |
| | (VR4 | FTDSDYQLPYVLGSAHEGCLPPFPADVEMIPCYGYLTLNDGSQAVGRSSFYCLEYFPSQMLRTGNNFQF |
| | insertion) | SYEFENVPFHSSYAHSQSLDRLMNPLIDQYLYYLSKTIN*QTLKFSVAGPSNMAVQGRNYIPGPSYRQQ |
| | [*- | RVSTTVTQNNNSEFAWPGASSWALNGRNSLMNPGPAMASHKEGEDRFFPLSGSLIFGKQGTGRDNVDAD |
| | linker- | KVMITNEEEIKTTNPVATESYGQVATNHQSACAQAQTGWVQNQGILPGMVWQDRDVYLQGPIWAKIPHT |
| | DARPin- | DGNFHPSPLMGGFGMKHPPPQILIKNTPVPADPPTAFNKDKLNSFITQYSTGQVSVEIEWELCKENSKR |
| | linker | WNPEIQYTSNYYKSNNVEFAVNTEGVYSEPRPIGTRYLTRNL |
| | inserted] | |
| | (VP2.VR4) | |
| 118 | AAV9 | MAPGKKRPVEQSPQEPDSSAGIGKSGAQPAKKRLNFGQTGDTESVPDPQPIGEPPAAPSGVGSLTMASG |
| | VP2 | GGAPVADNNEGADGVGSSSGNWHCDSQWLGDRVITTSTRTWALPTYNNHLYKQISNSTSGGSSNDNAYF |
| | only | GYSTPWGYFDFNRFHCHFSPRDWQRLINNNWGFRPKRLNFKLFNIQVKEVTDNNGVKTIANNLTSTVQV |
| | (VR4 | FTDSDYQLPYVLGSAHEGCLPPFPADVEMIPQYGYLTLNDGSQAVGRSSFYCLEYFPSQMLRTGNNFQF |
| | insertion | SYEFENVPFHSSYAHSQSLDRLMNPLIDQYLYYLSKTINGSGGGGSGGGGSGGGGSGGGGSGS* |
| | between | GSGGGGSGGGGSGGGGSGGGGSGSQTLKFSVAGPSNMAVQGRNYIPGPSYRQQRVSTTVTQNNNSEFAN |
| | linkers) | PGASSWALNGRNSLMNPGPAMASHKEGEDREEPLSGSLIFGKQGTGRDNVDADKVMITNEEEIKTTNPV |
| | [*- | ATESYGQVATNHQSAQAQAQTGWVQNQGILPGMVWQDRDVYLQGPIWAKIPHTDGNFEPSPLMGGFGMK |
| | DARPin- | HPPPQILIKNTPVPADPPTAFNKDKLNSFITCYSTGQVSVEIEWELQKENSKRWNPEIQYTSNYYKSNN |
| | inserted | VEFAVNTEGVYSEPRPIGTRYLTRNL |
| | between | |
| | GS24 | |
| | linkers] | |
| 119 | Capsid | MAADGYLPDWLEDNLSEGIREWWALKPGAPQPKANQQHQDNARGLVLPGYKYLGPGNGLDKGEPVNAAD |
| | L | AAALEHDKAYDQQLKAGDNPYLKYNHADAEFCERLKEDTSFGGNLGRAVFQAKKRLLEPLGLVEEAAKT |
| | VP2.VR4 | APGKKRPVEQSPQEPDSSAGIGKSGAQPAKKRLNFGQTGDTESVPDPQPIGEPPAAPSGVGSLTMASGG |
| | [*linker- | GAPVADNNEGADGVGSSSGNWHCDSQWLGDRVITTSTRIWALPTYNNHLYKQISNSTSGGSSNDNAYFG |
| | DARPin- | YSTPWGYFDFNRFHCHFSPRDWQRLINNNWGFRPKRLNFKLFNIQVKEVTDNNGVKTIANNLTSTVQVE |
| | linker | TDSDYQLPYVLGSAHEGCLPPFPADVFMIPQYGYLTLNDGSQAVGRSSFYCLEYEPSQMLRTGNNFQFS |
| | inserted | YEFENVPFHSSYAHSQSLDRLMNPLIDQYLYYLSKTINY |
| | after | CTLKFSVAGPSNMAVQGRNYIPGPSYRQQRVSTTVTQNNNSEFAWPGASSWALNGRNSLMNPGPAMASH |
| | N452] | KEGEDRFFPLSGSLIFGKQGTGRDNVDADKVMITNEEEIKTTNPVATESYGQVATNHQSAQACAQTGWV |
| | | CNQGILPGMVWQDRDVYLQGPIWAKIPHTDGNFHPSPLMGGFGMKHPPPQILIKNTPVPADPPTAENKD |
| | | KLNSFITQYSTGQVSVEIEWELQKENSKRWNPEIQYTSNYYKSNNVEFAVNTEGVYSEPRPIGTRYLTR |
| | | NL |
| 121 | AAV9. | MAADGYLPDWLEDNLSEGIREWWALKPGAPQPKANQQHQDNARGLVLPGYKYLGPGNGLDKGEPVNAAD |
| | AAA. | AAALEHDKAYDQQLKAGDNPYLKYNHADAEFGERLKEDTSFGGNLGRAVFQAKKRLLEPLGLVEEAAKT |
| | VP2-KO | APGKKRPVEQSPQEPDSSAGIGKSGAQPAKKRLNFGQTGDTESVPDPQPIGEPPAAPSGVGSLTMASGG |
| | (VP2 | GAPVADNNEGADGVGSSSGNWHCDSQWLGDRVITTSTRTWALPTYNNHLYKQTSNSTSGGSSNDNAYFG |
| | Initiation | YSTPWGYFDFNRFHCHFSPRDWQRLINNNWGFRPKRLNFKLFNIQVKEVTDNNGVKTIANNLTSTVQVE |
| | Site | TDSDYQLPYVLGSAHEGCLPPFPADVFMIPQYGYLTLNDGSQAVGRSSFYCLEYFPSQMLRTGNNFQFS |
| | Knock- | YEFENVPFHSSYAHSQSLDRLMNPLIDQYLYYLSKTINGSGQNQQTLKFSVAGPSNMAVQGRNYIPGPS |
| | out) | YRQQRVSTTVTQAAASEFAWPGASSWALNGRNSLMNPGPAMASHKEGEDRFFPLSGSLIFGKQGTGRDN |
| | | VDADKVMITNEEEIKTTNPVATESYGQVATNHQSAQAQAQTGWVQNQGILPGMVWQDRDVYLQGPIWAK |
| | | IPHTDGNFHPSPLMGGFGMKHPPPQILIKNTPVPADPPTAFNKDKLNSFITQYSTGQVSVEIEWELQKE |
| | | NSKRWNPEIQYTSNYYKSNNVEFAVNTEGVYSEPRPIGTRYLTRNL |
| 122 | AAV9. | [symbol] |
| | AAA. | [symbol] |
| | NVG07. | [symbol] |
| | VP2-KO | [symbol] |
| | (VP2 | [symbol] |
| | Initiation | [symbol] |
| | Site | [symbol] |
| | Knock- | [symbol] |
| | out) | [symbol] |
| | | [symbol] |
| | | [symbol] |
| 123 | AAVHu32. | [symbol] |
| | AAA. | [symbol] |
| | VP2-KO | [symbol] |
| | (VP2 | [symbol] |
| | Initiation | [symbol] |
| | Site | [symbol] |
| | Knock- | [symbol] |
| | out) | [symbol] |
| | | [symbol] |
| | | [symbol] |
| | | [symbol] |
| 124 | AAVHu32. | [symbol] |
| | AAA. | [symbol] |
| | NVG07. | [symbol] |
| | VP2-KO | [symbol] |
| | (VP2 | [symbol] |
| | Initiation | [symbol] |
| | Site | [symbol] |
| | Knock- | [symbol] |
| | out) | [symbol] |
| | | [symbol] |
| | | [symbol] |
| 125 | AAV9- | [symbol] |
| | VP2.VR8 | [symbol] |
| | [*- | [symbol] |
| | linker- | [symbol] |
| | DARPin- | [symbol] |
| | linker | [symbol] |
| | inserted] | [symbol] |
| | (VP2.VR8) | [symbol] |
| | | [symbol] |
| 128 | Capsid | [symbol] |
| | M | [symbol] |
| | VP2 | [symbol] |
| | VR4 | [symbol] |
| | [*DARPin- | [symbol] |
| | linker | [symbol] |
| | inserted | [symbol] |
| | before | [symbol] |
| | Q459] | [symbol] |
| 129 | Capsid | [symbol] |
| | M | [symbol] |
| | VP2 | [symbol] |
| | VR4 | [symbol] |
| | with | [symbol] |
| | long GS | [symbol] |
| | linkers | [symbol] |
| | [*DARPin- | [symbol] |
| | inserted | [symbol] |
| | before | [symbol] |
| | Q459] | |
| 130 | Capsid | [symbol] |
| | M | [symbol] |
| | VP2 | [symbol] |
| | VR4 | [symbol] |
| | with | [symbol] |
| | long GS | [symbol] |
| | linkers | [symbol] |
| | [*DARPin- | [symbol] |
| | inserted | [symbol] |
| | before | [symbol] |
| | Q459] | |
| 131 | Capsid | [symbol] |
| | M | [symbol] |
| | VP2 | [symbol] |
| | VR4 | [symbol] |
| | with | [symbol] |
| | long GS | [symbol] |
| | linkers | [symbol] |
| | [Xₙ] = | [symbol] |
| | DARPin | |
| | sequence | |
| | of n | |
| | amino | |
| | acid | |
| | length | |
| | inserted | |
| | 5′ of | |
| | Q459] | |
| 132 | Capsid | [symbol] |
| | N | [symbol] |
| | VP3 | [symbol] |
| | VR4 | [symbol] |
| | With | [symbol] |
| | long | [symbol] |
| | linkers | [symbol] |
| | underlin | [symbol] |
| | ed | [symbol] |
| | [Xₙ] = | |
| | DARPin | |
| | sequence | |
| | of n | |
| | amino | |
| | acid | |
| | length | |
| 133 | Capsid | [symbol] |
| | N | [symbol] |
| | VP3 | [symbol] |
| | VR4 | [symbol] |
| | With | [symbol] |
| | long | [symbol] |
| | linkers | [symbol] |
| | underlined | [symbol] |
| | [X126] = | [symbol] |
| | DARPin | |
| | sequence | |
| | of 126 | |
| | amino | |
| | acids in | |
| | length | |
| 134 | Wildtype | [symbol] |
| | AAV9 | [symbol] |
| | (VP1) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| 141 | Capsid | [symbol] |
| | O | [symbol] |
| | VP3 | [symbol] |
| | VR8 | [symbol] |
| | [*DARPin- | [symbol] |
| | linker | [symbol] |
| | insertion] | [symbol] |
| | | [symbol] |
| | | [symbol] |
| 142 | Capsid | [symbol] |
| | O | [symbol] |
| | VP3 | [symbol] |
| | VR8 | [symbol] |
| | with | [symbol] |
| | long | [symbol] |
| | linkers | [symbol] |
| | underlined | [symbol] |
| | [Xₙ] = | [symbol] |
| | DARPin | [symbol] |
| | sequence | |
| | of n | |
| | amino | |
| | acid | |
| | length | |

[symbol]

indicates data missing or illegible when filed

TABLE 8

| SEQ | | |
|---|---|---|
| ID | Brief | |
| NO | Description | Sequence |
| 101 | Capsid A | atggctgccgatggttatcttccagattggctcgaggacaaccttagtga |
| | sequence | aggaattcgcgagtggtgggctttgaaacctggagcccctcaacccaagg |
| | (trans): | caaatcaacaacatcaagacaacgctcgaggtcttgtgcttccgggttac |
| | Atropic | aaataccttggacccggcaacggactcgacaagggggagccggtcaacgc |
| | AAV9 | agcagacgcggcggccctcgagcacgacaaggcctacgaccagcagctca |
| | (VP1 only) | aggccggagacaacccgtacctcaagtacaaccacgccgacgccgagttc |
| | [AAV9.no | caggagcggctcaaagaagatacgtcttttgggggcaacctcgggcgagc |
| | VP2start.N | agtcttccaggccaaaaagaggcttcttgaacctcttggtctggttgagg |
| | 272A.496N | aagcggctaagaccgctcctggaaagaagaggcctgtagagcagtctcct |
| | NNLAAA4 | caggaaccggactcctccgcgggtattggcaaatcgggtgcacagcccgc |
| | 98] | taaaaagagactcaatttcggtcagactggcgacacagagtcagtcccag |
| | | accctcaaccaatcggagaacctcccgcagccccctcaggtgtgggatct |
| | | cttacaatggcttcaggtggtggcgcaccagtggcagacaataacgaagg |
| | | tgccgatggagtgggtagttcctcgggaaattggcattgcgattcccaat |
| | | ggctgggggacagagtcatcaccaccagcacccgaacctgggccctgccc |
| | | acctacaacaatcacctctacaagcaaatctccaacagcacatctggagg |
| | | atcttcaaatgacgccgcctacttcggctacagcaccccctgggggtatt |
| | | ttgacttcaacagattccactgccacttctcaccacgtgactggcagcga |
| | | ctcatcaacaacaactggggattccggcctaagcgactcaacttcaagct |
| | | cttcaacattcaggtcaaagaggttacggacaacaatggagtcaagacca |
| | | tcgccaataaccttaccagcacggtccaggtcttcacggactcagactat |
| | | cagctcccgtacgtgctcgggtcggctcacgagggctgcctcccgccgtt |
| | | cccagcggacgttttcatgattcctcagtacgggtatctgacgcttaatg |
| | | atggaagccaggccgtgggtcgttcgtccttttactgcctggaatatttc |
| | | ccgtcgcaaatgctaagaacgggtaacaacttccagttcagctacgagtt |
| | | tgagaacgtacctttccatagcagctacgctcacagccaaagcctggacc |
| | | gactaatgaatccactcatcgaccaatacttgtactatctctcaaagact |
| | | attaacggttctggacagaatcaacaaacgctaaaattcagtgtggccgg |
| | | acccagcaacatggctgtccagggaagaaactacatacctggacccagct |
| | | accgacaacaacgtgtctcaaccactgtgactcaagcagcggcaagcgaa |
| | | tttgcttggcctggagcttcttcttgggctctcaatggacgtaatagctt |
| | | gatgaatcctggacctgctatggccagccacaaagaaggagaggaccgtt |
| | | tctttcctttgtctggatctttaatttttggcaaacaaggaactggaaga |
| | | gacaacgtggatgcggacaaagtcatgataaccaacgaagaagaaattaa |
| | | aactactaacccggtagcaacggagtcctatggacaagtggccacaaacc |
| | | accagagtgcccaagcacaggcgcagaccggctgggttcaaaaccaagga |
| | | atacttccgggtatggtttggcaggacagagatgtgtacctgcaaggacc |
| | | catttgggccaaaattcctcacacggacggcaactttcacccttctccgc |
| | | tgatgggagggtttggaatgaagcacccgcctcctcagatcctcatcaaa |
| | | aacacacctgtacctgcggatcctccaacggccttcaacaaggacaagct |
| | | gaactctttcatcacccagtattctactggccaagtcagcgtggagatcg |
| | | agtgggagctgcagaaggaaaacagcaagcgctggaacccggagatccag |
| | | tacacttccaactattacaagtctaataatgttgaatttgctgttaatac |
| | | tgaaggtgtatatagtgaaccccgccccattggcaccagatacctgactc |
| | | gtaatctgtaa |
| 102 | Capsid A | atgccggggttttacgagattgtgattaaggtccccagcgaccttgacga |
| | sequence | gcatctgcccggcatttctgacagctttgtgaactgggtggccgagaagg |
| | (trans): | aatgggagttgccgccagattctgacatggatctgaatctgattgagcag |
| | Rep2/Cap | gcacccctgaccgtggccgagaagctgcagcgcgactttctgacggaatg |
| | Atropic | gcgccgtgtgagtaaggccccggaggctcttttctttgtgcaatttgaga |
| | AAV9 | agggagagagctacttccacatgcacgtgctcgtggaaaccaccggggtg |
| | (VP1) | aaatccatggttttgggacgtttcctgagtcagattcgcgaaaaactgat |
| | [AAV9.no | tcagagaatttaccgcgggatcgagccgactttgccaaactggttcgcgg |
| | VP2start.N | tcacaaagaccagaaatggcgccggaggcgggaacaaggtggtggatgag |
| | 272A.496N | tgctacatccccaattacttgctccccaaaacccagcctgagctccagtg |
| | NNLAAA4 | ggcgtggactaatatggaacagtatttaagcgcctgtttgaatctcacgg |
| | 98] | agcgtaaacggttggtggcgcagcatctgacgcacgtgtcgcagacgcag |
| | | gagcagaacaaagagaatcagaatcccaattctgatgcgccggtgatcag |
| | | atcaaaaacttcagccaggtacatggagctggtcgggtggctcgtggaca |
| | | aggggattacctcggagaagcagtggatccaggaggaccaggcctcatac |
| | | atctccttcaatgcggcctccaactcgcggtcccaaatcaaggctgcctt |
| | | ggacaatgcgggaaagattatgagcctgactaaaaccgcccccgactacc |
| | | tggtgggccagcagcccgtggaggacatttccagcaatcggatttataaa |
| | | attttggaactaaacgggtacgatccccaatatgcggcttccgtctttct |
| | | gggatgggccacgaaaaagttcggcaagaggaacaccatctggctgtttg |
| | | ggcctgcaactaccgggaagaccaacatcgcggaggccatagcccacact |
| | | gtgcccttctacgggtgcgtaaactggaccaatgagaactttcccttcaa |
| | | cgactgtgtcgacaagatggtgatctggtgggaggaggggaagatgaccg |
| | | ccaaggtcgtggagtcggccaaagccattctcggaggaagcaaggtgcgc |
| | | gtggaccagaaatgcaagtcctcggcccagatagacccgactcccgtgat |
| | | cgtcacctccaacaccaacatgtgcgccgtgattgacgggaactcaacga |
| | | ccttcgaacaccagcagccgttgcaagaccggatgttcaaatttgaactc |
| | | acccgccgtctggatcatgactttgggaaggtcaccaagcaggaagtcaa |
| | | agactttttccggtgggcaaaggatcacgtggttgaggtggagcatgaat |
| | | tctacgtcaaaaagggtggagccaagaaaagacccgcccccagtgacgca |
| | | gatataagtgagcccaaacgggtgcgcgagtcagttgcgcagccatcgac |
| | | gtcagacgcggaagcttcgatcaactacgcagacaggtaccaaaacaaat |
| | | gttctcgtcacgtgggcatgaatctgatgctgtttccctgcagacaatgc |
| | | gagagaatgaatcagaattcaaatatctgcttcactcacggacagaaaga |
| | | ctgtttagagtgctttcccgtgtcagaatctcaacccgtttctgtcgtca |
| | | aaaaggcgtatcagaaactgtgctacattcatcatatcatgggaaaggtg |
| | | ccagacgcttgcactgcctgcgatctggtcaatgtggatttggatgactg |
| | | catctttgaacaataaatgacttaaaccaggtatggctgccgatggttat |
| | | cttccagattggctcgaggacaaccttagtgaaggaattcgcgagtggtg |
| | | ggctttgaaacctggagcccctcaacccaaggcaaatcaacaacatcaag |
| | | acaacgctcgaggtcttgtgcttccgggttacaaataccttggacccggc |
| | | aacggactcgacaagggggagccggtcaacgcagcagacgcggcggccct |
| | | cgagcacgacaaggcctacgaccagcagctcaaggccggagacaacccgt |
| | | acctcaagtacaaccacgccgacgccgagttccaggagcggctcaaagaa |
| | | gatacgtcttttgggggcaacctcgggcgagcagtcttccaggccaaaaa |
| | | gaggcttcttgaacctcttggtctggttgaggaagcggctaagaccgctc |
| | | ctggaaagaagaggcctgtagagcagtctcctcaggaaccggactcctcc |
| | | gcgggtattggcaaatcgggtgcacagcccgctaaaaagagactcaattt |
| | | cggtcagactggcgacacagagtcagtcccagaccctcaaccaatcggag |
| | | aacctcccgcagccccctcaggtgtgggatctcttacaatggcttcaggt |
| | | ggtggcgcaccagtggcagacaataacgaaggtgccgatggagtgggtag |
| | | ttcctcgggaaattggcattgcgattcccaatggctgggggacagagtca |
| | | tcaccaccagcacccgaacctgggccctgcccacctacaacaatcacctc |
| | | tacaagcaaatctccaacagcacatctggaggatcttcaaatgacgccgc |
| | | ctacttcggctacagcaccccctgggggtattttgacttcaacagattcc |
| | | actgccacttctcaccacgtgactggcagcgactcatcaacaacaactgg |
| | | ggattccggcctaagcgactcaacttcaagctcttcaacattcaggtcaa |
| | | agaggttacggacaacaatggagtcaagaccatcgccaataaccttacca |
| | | gcacggtccaggtcttcacggactcagactatcagctcccgtacgtgctc |
| | | gggtcggctcacgagggctgcctcccgccgttcccagcggacgttttcat |
| | | gattcctcagtacgggtatctgacgcttaatgatggaagccaggccgtgg |
| | | gtcgttcgtccttttactgcctggaatatttcccgtcgcaaatgctaaga |
| | | acgggtaacaacttccagttcagctacgagtttgagaacgtacctttcca |
| | | tagcagctacgctcacagccaaagcctggaccgactaatgaatccactca |
| | | tcgaccaatacttgtactatctctcaaagactattaacggttctggacag |
| | | aatcaacaaacgctaaaattcagtgtggccggacccagcaacatggctgt |
| | | ccagggaagaaactacatacctggacccagctaccgacaacaacgtgtct |
| | | caaccactgtgactcaagcagcggcaagcgaatttgcttggcctggagct |
| | | tcttcttgggctctcaatggacgtaatagcttgatgaatcctggacctgc |
| | | tatggccagccacaaagaaggagaggaccgtttctttcctttgtctggat |
| | | ctttaatttttggcaaacaaggaactggaagagacaacgtggatgcggac |
| | | aaagtcatgataaccaacgaagaagaaattaaaactactaacccggtagc |
| | | aacggagtcctatggacaagtggccacaaaccaccagagtgcccaagcac |
| | | aggcgcagaccggctgggttcaaaaccaaggaatacttccgggtatggtt |
| | | tggcaggacagagatgtgtacctgcaaggacccatttgggccaaaattcc |
| | | tcacacggacggcaactttcacccttctccgctgatgggagggtttggaa |
| | | tgaagcacccgcctcctcagatcctcatcaaaaacacacctgtacctgcg |
| | | gatcctccaacggccttcaacaaggacaagctgaactctttcatcaccca |
| | | gtattctactggccaagtcagcgtggagatcgagtgggagctgcagaagg |
| | | aaaacagcaagcgctggaacccggagatccagtacacttccaactattac |
| | | aagtctaataatgttgaatttgctgttaatactgaaggtgtatatagtga |
| | | accccgccccattggcaccagatacctgactcgtaatctgtaa |
| 103 | Capsid A | ggaggctctgctcctggaaagaagaggcctgtagagcagtctcctcagga |
| | (secondary): | accggactcctccgcgggtattggcaaatcgggtgcacagcccgctaaaa |
| | Atropic | agagactcaatttcggtcagactggcgacacagagtcagtcccagaccct |
| | AAV9 VP2 | caaccaatcggagaacctcccgcagccccctcaggtgtgggatctcttac |
| | having - | aatggcttcaggtggtggcgcaccagtggcagacaataacgaaggtgccg |
| | GGS at N- | atggagtgggtagttcctcgggaaattggcattgcgattcccaatggctg |
| | Term | ggggacagagtcatcaccaccagcacccgaacctgggccctgcccaccta |
| | | caacaatcacctctacaagcaaatctccaacagcacatctggaggatctt |
| | | caaatgacgccgcctacttcggctacagcaccccctgggggtattttgac |
| | | ttcaacagattccactgccacttctcaccacgtgactggcagcgactcat |
| | | caacaacaactggggattccggcctaagcgactcaacttcaagctcttca |
| | | acattcaggtcaaagaggttacggacaacaatggagtcaagaccatcgcc |
| | | aataaccttaccagcacggtccaggtcttcacggactcagactatcagct |
| | | cccgtacgtgctcgggtcggctcacgagggctgcctcccgccgttcccag |
| | | cggacgttttcatgattcctcagtacgggtatctgacgcttaatgatgga |
| | | agccaggccgtgggtcgttcgtccttttactgcctggaatatttcccgtc |
| | | gcaaatgctaagaacgggtaacaacttccagttcagctacgagtttgaga |
| | | acgtacctttccatagcagctacgctcacagccaaagcctggaccgacta |
| | | atgaatccactcatcgaccaatacttgtactatctctcaaagactattaa |
| | | cggttctggacagaatcaacaaacgctaaaattcagtgtggccggaccca |
| | | gcaacatggctgtccagggaagaaactacatacctggacccagctaccga |
| | | caacaacgtgtctcaaccactgtgactcaagcagcggcaagcgaatttgc |
| | | ttggcctggagcttcttcttgggctctcaatggacgtaatagcttgatga |
| | | atcctggacctgctatggccagccacaaagaaggagaggaccgtttcttt |
| | | cctttgtctggatctttaatttttggcaaacaaggaactggaagagacaa |
| | | cgtggatgcggacaaagtcatgataaccaacgaagaagaaattaaaacta |
| | | ctaacccggtagcaacggagtcctatggacaagtggccacaaaccaccag |
| | | agtgcccaagcacaggcgcagaccggctgggttcaaaaccaaggaatact |
| | | tccgggtatggtttggcaggacagagatgtgtacctgcaaggacccattt |
| | | gggccaaaattcctcacacggacggcaactttcacccttctccgctgatg |
| | | ggagggtttggaatgaagcacccgcctcctcagatcctcatcaaaaacac |
| | | acctgtacctgcggatcctccaacggccttcaacaaggacaagctgaact |
| | | ctttcatcacccagtattctactggccaagtcagcgtggagatcgagtgg |
| | | gagctgcagaaggaaaacagcaagcgctggaacccggagatccagtacac |
| | | ttccaactattacaagtctaataatgttgaatttgctgttaatactgaag |
| | | gtgtatatagtgaaccccgccccattggcaccagatacctgactcgtaat |
| | | ctgtaa |
| 104 | Capsid A | atggacctgggcaagaagctgcttgaggccgctagagccggccagaacga |
| | (secondary) | cgaggtgcggatcctgatggccaacggcgccgacgtgaacgccatcgaca |
| | Atropic | tggccggaagaacacctctgcacctcgccgcttggtccggccacctggaa |
| | AAV9 VP2 | atcgtggaagtgctgctgaagtacgacgccgatgtgaacgccaccgacca |
| | having | cttcggcctgacccctctgcacctggccgccagcgacggccacctggaca |
| | DARPin | tcgccgaggtgctgcagaaggccgccggaggctctgctcctggaaagaag |
| | (GluA4)- | aggcctgtagagcagtctcctcaggaaccggactcctccgcgggtattgg |
| | GGS at N- | caaatcgggtgcacagcccgctaaaaagagactcaatttcggtcagactg |
| | Term | gcgacacagagtcagtcccagaccctcaaccaatcggagaacctcccgca |
| | | gccccctcaggtgtgggatctcttacaatggcttcaggtggtggcgcacc |
| | | agtggcagacaataacgaaggtgccgatggagtgggtagttcctcgggaa |
| | | attggcattgcgattcccaatggctgggggacagagtcatcaccaccagc |
| | | acccgaacctgggccctgcccacctacaacaatcacctctacaagcaaat |
| | | ctccaacagcacatctggaggatcttcaaatgacgccgcctacttcggct |
| | | acagcaccccctgggggtattttgacttcaacagattccactgccacttc |
| | | tcaccacgtgactggcagcgactcatcaacaacaactggggattccggcc |
| | | taagcgactcaacttcaagctcttcaacattcaggtcaaagaggttacgg |
| | | acaacaatggagtcaagaccatcgccaataaccttaccagcacggtccag |
| | | gtcttcacggactcagactatcagctcccgtacgtgctcgggtcggctca |
| | | cgagggctgcctcccgccgttcccagcggacgttttcatgattcctcagt |
| | | acgggtatctgacgcttaatgatggaagccaggccgtgggtcgttcgtcc |
| | | ttttactgcctggaatatttcccgtcgcaaatgctaagaacgggtaacaa |
| | | cttccagttcagctacgagtttgagaacgtacctttccatagcagctacg |
| | | ctcacagccaaagcctggaccgactaatgaatccactcatcgaccaatac |
| | | ttgtactatctctcaaagactattaacggttctggacagaatcaacaaac |
| | | gctaaaattcagtgtggccggacccagcaacatggctgtccagggaagaa |
| | | actacatacctggacccagctaccgacaacaacgtgtctcaaccactgtg |
| | | actcaagcagcggcaagcgaatttgcttggcctggagcttcttcttgggc |
| | | tctcaatggacgtaatagcttgatgaatcctggacctgctatggccagcc |
| | | acaaagaaggagaggaccgtttctttcctttgtctggatctttaattttt |
| | | ggcaaacaaggaactggaagagacaacgtggatgcggacaaagtcatgat |
| | | aaccaacgaagaagaaattaaaactactaacccggtagcaacggagtcct |
| | | atggacaagtggccacaaaccaccagagtgcccaagcacaggcgcagacc |
| | | ggctgggttcaaaaccaaggaatacttccgggtatggtttggcaggacag |
| | | agatgtgtacctgcaaggacccatttgggccaaaattcctcacacggacg |
| | | gcaactttcacccttctccgctgatgggagggtttggaatgaagcacccg |
| | | cctcctcagatcctcatcaaaaacacacctgtacctgcggatcctccaac |
| | | ggccttcaacaaggacaagctgaactctttcatcacccagtattctactg |
| | | gccaagtcagcgtggagatcgagtgggagctgcagaaggaaaacagcaag |
| | | cgctggaacccggagatccagtacacttccaactattacaagtctaataa |
| | | tgttgaatttgctgttaatactgaaggtgtatatagtgaaccccgcccca |
| | | ttggcaccagatacctgactcgtaatctgtaa |
| 105 | Capsid B | atggctgccgatggttatcttccagattggctcgaggacaaccttagtga |
| | trans seq: | aggaattcgcgagtggtgggctttgaaacctggagcccctcaacccaagg |
| | wt AAV9 | caaatcaacaacatcaagacaacgctcgaggtcttgtgcttccgggttac |
| | (VP1) | aaataccttggacccggcaacggactcgacaagggggagccggtcaacgc |
| | [no VP2 | agcagacgcggcggccctcgagcacgacaaggcctacgaccagcagctca |
| | start] | aggccggagacaacccgtacctcaagtacaaccacgccgacgccgagttc |
| | | caggagcggctcaaagaagatacgtcttttgggggcaacctcgggcgagc |
| | | agtcttccaggccaaaaagaggcttcttgaacctcttggtctggttgagg |
| | | aagcggctaagaccgctcctggaaagaagaggcctgtagagcagtctcct |
| | | caggaaccggactcctccgcgggtattggcaaatcgggtgcacagcccgc |
| | | taaaaagagactcaatttcggtcagactggcgacacagagtcagtcccag |
| | | accctcaaccaatcggagaacctcccgcagccccctcaggtgtgggatct |
| | | cttacaatggcttcaggtggtggcgcaccagtggcagacaataacgaagg |
| | | tgccgatggagtgggtagttcctcgggaaattggcattgcgattcccaat |
| | | ggctgggggacagagtcatcaccaccagcacccgaacctgggccctgccc |
| | | acctacaacaatcacctctacaagcaaatctccaacagcacatctggagg |
| | | atcttcaaatgacaacgcctacttcggctacagcaccccctgggggtatt |
| | | ttgacttcaacagattccactgccacttctcaccacgtgactggcagcga |
| | | ctcatcaacaacaactggggattccggcctaagcgactcaacttcaagct |
| | | cttcaacattcaggtcaaagaggttacggacaacaatggagtcaagacca |
| | | tcgccaataaccttaccagcacggtccaggtcttcacggactcagactat |
| | | cagctcccgtacgtgctcgggtcggctcacgagggctgcctcccgccgtt |
| | | cccagcggacgttttcatgattcctcagtacgggtatctgacgcttaatg |
| | | atggaagccaggccgtgggtcgttcgtccttttactgcctggaatatttc |
| | | ccgtcgcaaatgctaagaacgggtaacaacttccagttcagctacgagtt |
| | | tgagaacgtacctttccatagcagctacgctcacagccaaagcctggacc |
| | | gactaatgaatccactcatcgaccaatacttgtactatctctcaaagact |
| | | attaacggttctggacagaatcaacaaacgctaaaattcagtgtggccgg |
| | | acccagcaacatggctgtccagggaagaaactacatacctggacccagct |
| | | accgacaacaacgtgtctcaaccactgtgactcaaaacaacaacagcgaa |
| | | tttgcttggcctggagcttcttcttgggctctcaatggacgtaatagctt |
| | | gatgaatcctggacctgctatggccagccacaaagaaggagaggaccgtt |
| | | tctttcctttgtctggatctttaatttttggcaaacaaggaactggaaga |
| | | gacaacgtggatgcggacaaagtcatgataaccaacgaagaagaaattaa |
| | | aactactaacccggtagcaacggagtcctatggacaagtggccacaaacc |
| | | accagagtgcccaagcacaggcgcagaccggctgggttcaaaaccaagga |
| | | atacttccgggtatggtttggcaggacagagatgtgtacctgcaaggacc |
| | | catttgggccaaaattcctcacacggacggcaactttcacccttctccgc |
| | | tgatgggagggtttggaatgaagcacccgcctcctcagatcctcatcaaa |
| | | aacacacctgtacctgcggatcctccaacggccttcaacaaggacaagct |
| | | gaactctttcatcacccagtattctactggccaagtcagcgtggagatcg |
| | | agtgggagctgcagaaggaaaacagcaagcgctggaacccggagatccag |
| | | tacacttccaactattacaagtctaataatgttgaatttgctgttaatac |
| | | tgaaggtgtatatagtgaaccccgccccattggcaccagatacctgactc |
| | | gtaatctgtaa |
| 106 | Capsid B | atgccggggttttacgagattgtgattaaggtccccagcgaccttgacga |
| | trans seq: | gcatctgcccggcatttctgacagctttgtgaactgggtggccgagaagg |
| | Rep2/Cap | aatgggagttgccgccagattctgacatggatctgaatctgattgagcag |
| | AAV9 | gcacccctgaccgtggccgagaagctgcagcgcgactttctgacggaatg |
| | (VP1) | gcgccgtgtgagtaaggccccggaggctcttttctttgtgcaatttgaga |
| | [no VP2 | agggagagagctacttccacatgcacgtgctcgtggaaaccaccggggtg |
| | start] | aaatccatggttttgggacgtttcctgagtcagattcgcgaaaaactgat |
| | | tcagagaatttaccgcgggatcgagccgactttgccaaactggttcgcgg |
| | | tcacaaagaccagaaatggcgccggaggcgggaacaaggtggtggatgag |
| | | tgctacatccccaattacttgctccccaaaacccagcctgagctccagtg |
| | | ggcgtggactaatatggaacagtatttaagcgcctgtttgaatctcacgg |
| | | agcgtaaacggttggtggcgcagcatctgacgcacgtgtcgcagacgcag |
| | | gagcagaacaaagagaatcagaatcccaattctgatgcgccggtgatcag |
| | | atcaaaaacttcagccaggtacatggagctggtcgggtggctcgtggaca |
| | | aggggattacctcggagaagcagtggatccaggaggaccaggcctcatac |
| | | atctccttcaatgcggcctccaactcgcggtcccaaatcaaggctgcctt |
| | | ggacaatgcgggaaagattatgagcctgactaaaaccgcccccgactacc |
| | | tggtgggccagcagcccgtggaggacatttccagcaatcggatttataaa |
| | | attttggaactaaacgggtacgatccccaatatgcggcttccgtctttct |
| | | gggatgggccacgaaaaagttcggcaagaggaacaccatctggctgtttg |
| | | ggcctgcaactaccgggaagaccaacatcgcggaggccatagcccacact |
| | | gtgcccttctacgggtgcgtaaactggaccaatgagaactttcccttcaa |
| | | cgactgtgtcgacaagatggtgatctggtgggaggaggggaagatgaccg |
| | | ccaaggtcgtggagtcggccaaagccattctcggaggaagcaaggtgcgc |
| | | gtggaccagaaatgcaagtcctcggcccagatagacccgactcccgtgat |
| | | cgtcacctccaacaccaacatgtgcgccgtgattgacgggaactcaacga |
| | | ccttcgaacaccagcagccgttgcaagaccggatgttcaaatttgaactc |
| | | acccgccgtctggatcatgactttgggaaggtcaccaagcaggaagtcaa |
| | | agactttttccggtgggcaaaggatcacgtggttgaggtggagcatgaat |
| | | tctacgtcaaaaagggtggagccaagaaaagacccgcccccagtgacgca |
| | | gatataagtgagcccaaacgggtgcgcgagtcagttgcgcagccatcgac |
| | | gtcagacgcggaagcttcgatcaactacgcagacaggtaccaaaacaaat |
| | | gttctcgtcacgtgggcatgaatctgatgctgtttccctgcagacaatgc |
| | | gagagaatgaatcagaattcaaatatctgcttcactcacggacagaaaga |
| | | ctgtttagagtgctttcccgtgtcagaatctcaacccgtttctgtcgtca |
| | | aaaaggcgtatcagaaactgtgctacattcatcatatcatgggaaaggtg |
| | | ccagacgcttgcactgcctgcgatctggtcaatgtggatttggatgactg |
| | | catctttgaacaataaatgacttaaaccaggtatggctgccgatggttat |
| | | cttccagattggctcgaggacaaccttagtgaaggaattcgcgagtggtg |
| | | ggctttgaaacctggagcccctcaacccaaggcaaatcaacaacatcaag |
| | | acaacgctcgaggtcttgtgcttccgggttacaaataccttggacccggc |
| | | aacggactcgacaagggggagccggtcaacgcagcagacgcggcggccct |
| | | cgagcacgacaaggcctacgaccagcagctcaaggccggagacaacccgt |
| | | acctcaagtacaaccacgccgacgccgagttccaggagcggctcaaagaa |
| | | gatacgtcttttgggggcaacctcgggcgagcagtcttccaggccaaaaa |
| | | gaggcttcttgaacctcttggtctggttgaggaagcggctaagaccgctc |
| | | ctggaaagaagaggcctgtagagcagtctcctcaggaaccggactcctcc |
| | | gcgggtattggcaaatcgggtgcacagcccgctaaaaagagactcaattt |
| | | cggtcagactggcgacacagagtcagtcccagaccctcaaccaatcggag |
| | | aacctcccgcagccccctcaggtgtgggatctcttacaatggcttcaggt |
| | | ggtggcgcaccagtggcagacaataacgaaggtgccgatggagtgggtag |
| | | ttcctcgggaaattggcattgcgattcccaatggctgggggacagagtca |
| | | tcaccaccagcacccgaacctgggccctgcccacctacaacaatcacctc |
| | | tacaagcaaatctccaacagcacatctggaggatcttcaaatgacaacgc |
| | | ctacttcggctacagcaccccctgggggtattttgacttcaacagattcc |
| | | actgccacttctcaccacgtgactggcagcgactcatcaacaacaactgg |
| | | ggattccggcctaagcgactcaacttcaagctcttcaacattcaggtcaa |
| | | agaggttacggacaacaatggagtcaagaccatcgccaataaccttacca |
| | | gcacggtccaggtcttcacggactcagactatcagctcccgtacgtgctc |
| | | gggtcggctcacgagggctgcctcccgccgttcccagcggacgttttcat |
| | | gattcctcagtacgggtatctgacgcttaatgatggaagccaggccgtgg |
| | | gtcgttcgtccttttactgcctggaatatttcccgtcgcaaatgctaaga |
| | | acgggtaacaacttccagttcagctacgagtttgagaacgtacctttcca |
| | | tagcagctacgctcacagccaaagcctggaccgactaatgaatccactca |
| | | tcgaccaatacttgtactatctctcaaagactattaacggttctggacag |
| | | aatcaacaaacgctaaaattcagtgtggccggacccagcaacatggctgt |
| | | ccagggaagaaactacatacctggacccagctaccgacaacaacgtgtct |
| | | caaccactgtgactcaaaacaacaacagcgaatttgcttggcctggagct |
| | | tcttcttgggctctcaatggacgtaatagcttgatgaatcctggacctgc |
| | | tatggccagccacaaagaaggagaggaccgtttctttcctttgtctggat |
| | | ctttaatttttggcaaacaaggaactggaagagacaacgtggatgcggac |
| | | aaagtcatgataaccaacgaagaagaaattaaaactactaacccggtagc |
| | | aacggagtcctatggacaagtggccacaaaccaccagagtgcccaagcac |
| | | aggcgcagaccggctgggttcaaaaccaaggaatacttccgggtatggtt |
| | | tggcaggacagagatgtgtacctgcaaggacccatttgggccaaaattcc |
| | | tcacacggacggcaactttcacccttctccgctgatgggagggtttggaa |
| | | tgaagcacccgcctcctcagatcctcatcaaaaacacacctgtacctgcg |
| | | gatcctccaacggccttcaacaaggacaagctgaactctttcatcaccca |
| | | gtattctactggccaagtcagcgtggagatcgagtgggagctgcagaagg |
| | | aaaacagcaagcgctggaacccggagatccagtacacttccaactattac |
| | | aagtctaataatgttgaatttgctgttaatactgaaggtgtatatagtga |
| | | accccgccccattggcaccagatacctgactcgtaatctgtaa |
| 107 | Capsid B | ggaggctctgctcctggaaagaagaggcctgtagagcagtctcctcagga |
| | (secondary): | accggactcctccgcgggtattggcaaatcgggtgcacagcccgctaaaa |
| | wtAAV9- | agagactcaatttcggtcagactggcgacacagagtcagtcccagaccct |
| | DARPin- | caaccaatcggagaacctcccgcagccccctcaggtgtgggatctcttac |
| | GGS N- | aatggcttcaggtggtggcgcaccagtggcagacaataacgaaggtgccg |
| | term | atggagtgggtagttcctcgggaaattggcattgcgattcccaatggctg |
| | | ggggacagagtcatcaccaccagcacccgaacctgggccctgcccaccta |
| | | caacaatcacctctacaagcaaatctccaacagcacatctggaggatctt |
| | | caaatgacaacgcctacttcggctacagcaccccctgggggtattttgac |
| | | ttcaacagattccactgccacttctcaccacgtgactggcagcgactcat |
| | | caacaacaactggggattccggcctaagcgactcaacttcaagctcttca |
| | | acattcaggtcaaagaggttacggacaacaatggagtcaagaccatcgcc |
| | | aataaccttaccagcacggtccaggtcttcacggactcagactatcagct |
| | | cccgtacgtgctcgggtcggctcacgagggctgcctcccgccgttcccag |
| | | cggacgttttcatgattcctcagtacgggtatctgacgcttaatgatgga |
| | | agccaggccgtgggtcgttcgtccttttactgcctggaatatttcccgtc |
| | | gcaaatgctaagaacgggtaacaacttccagttcagctacgagtttgaga |
| | | acgtacctttccatagcagctacgctcacagccaaagcctggaccgacta |
| | | atgaatccactcatcgaccaatacttgtactatctctcaaagactattaa |
| | | cggttctggacagaatcaacaaacgctaaaattcagtgtggccggaccca |
| | | gcaacatggctgtccagggaagaaactacatacctggacccagctaccga |
| | | caacaacgtgtctcaaccactgtgactcaaaacaacaacagcgaatttgc |
| | | ttggcctggagcttcttcttgggctctcaatggacgtaatagcttgatga |
| | | atcctggacctgctatggccagccacaaagaaggagaggaccgtttcttt |
| | | cctttgtctggatctttaatttttggcaaacaaggaactggaagagacaa |
| | | cgtggatgcggacaaagtcatgataaccaacgaagaagaaattaaaacta |
| | | ctaacccggtagcaacggagtcctatggacaagtggccacaaaccaccag |
| | | agtgcccaagcacaggcgcagaccggctgggttcaaaaccaaggaatact |
| | | tccgggtatggtttggcaggacagagatgtgtacctgcaaggacccattt |
| | | gggccaaaattcctcacacggacggcaactttcacccttctccgctgatg |
| | | ggagggtttggaatgaagcacccgcctcctcagatcctcatcaaaaacac |
| | | acctgtacctgcggatcctccaacggccttcaacaaggacaagctgaact |
| | | ctttcatcacccagtattctactggccaagtcagcgtggagatcgagtgg |
| | | gagctgcagaaggaaaacagcaagcgctggaacccggagatccagtacac |
| | | ttccaactattacaagtctaataatgttgaatttgctgttaatactgaag |
| | | gtgtatatagtgaaccccgccccattggcaccagatacctgactcgtaat |
| | | ctgtaa |
| 108 | Capsid B | atggacctgggcaagaagctgcttgaggccgctagagccggccagaacga |
| | (secondary): | cgaggtgcggatcctgatggccaacggcgccgacgtgaacgccatcgaca |
| | with | tggccggaagaacacctctgcacctcgccgcttggtccggccacctggaa |
| | wtAAV9 | atcgtggaagtgctgctgaagtacgacgccgatgtgaacgccaccgacca |
| | DARPin | cttcggcctgacccctctgcacctggccgccagcgacggccacctggaca |
| | (GluA4) | tcgccgaggtgctgcagaaggccgccggaggctctgctcctggaaagaag |
| | | aggcctgtagagcagtctcctcaggaaccggactcctccgcgggtattgg |
| | | caaatcgggtgcacagcccgctaaaaagagactcaatttcggtcagactg |
| | | gcgacacagagtcagtcccagaccctcaaccaatcggagaacctcccgca |
| | | gccccctcaggtgtgggatctcttacaatggcttcaggtggtggcgcacc |
| | | agtggcagacaataacgaaggtgccgatggagtgggtagttcctcgggaa |
| | | attggcattgcgattcccaatggctgggggacagagtcatcaccaccagc |
| | | acccgaacctgggccctgcccacctacaacaatcacctctacaagcaaat |
| | | ctccaacagcacatctggaggatcttcaaatgacaacgcctacttcggct |
| | | acagcaccccctgggggtattttgacttcaacagattccactgccacttc |
| | | tcaccacgtgactggcagcgactcatcaacaacaactggggattccggcc |
| | | taagcgactcaacttcaagctcttcaacattcaggtcaaagaggttacgg |
| | | acaacaatggagtcaagaccatcgccaataaccttaccagcacggtccag |
| | | gtcttcacggactcagactatcagctcccgtacgtgctcgggtcggctca |
| | | cgagggctgcctcccgccgttcccagcggacgttttcatgattcctcagt |
| | | acgggtatctgacgcttaatgatggaagccaggccgtgggtcgttcgtcc |
| | | ttttactgcctggaatatttcccgtcgcaaatgctaagaacgggtaacaa |
| | | cttccagttcagctacgagtttgagaacgtacctttccatagcagctacg |
| | | ctcacagccaaagcctggaccgactaatgaatccactcatcgaccaatac |
| | | ttgtactatctctcaaagactattaacggttctggacagaatcaacaaac |
| | | gctaaaattcagtgtggccggacccagcaacatggctgtccagggaagaa |
| | | actacatacctggacccagctaccgacaacaacgtgtctcaaccactgtg |
| | | actcaaaacaacaacagcgaatttgcttggcctggagcttcttcttgggc |
| | | tctcaatggacgtaatagcttgatgaatcctggacctgctatggccagcc |
| | | acaaagaaggagaggaccgtttctttcctttgtctggatctttaattttt |
| | | ggcaaacaaggaactggaagagacaacgtggatgcggacaaagtcatgat |
| | | aaccaacgaagaagaaattaaaactactaacccggtagcaacggagtcct |
| | | atggacaagtggccacaaaccaccagagtgcccaagcacaggcgcagacc |
| | | ggctgggttcaaaaccaaggaatacttccgggtatggtttggcaggacag |
| | | agatgtgtacctgcaaggacccatttgggccaaaattcctcacacggacg |
| | | gcaactttcacccttctccgctgatgggagggtttggaatgaagcacccg |
| | | cctcctcagatcctcatcaaaaacacacctgtacctgcggatcctccaac |
| | | ggccttcaacaaggacaagctgaactctttcatcacccagtattctactg |
| | | gccaagtcagcgtggagatcgagtgggagctgcagaaggaaaacagcaag |
| | | cgctggaacccggagatccagtacacttccaactattacaagtctaataa |
| | | tgttgaatttgctgttaatactgaaggtgtatatagtgaaccccgcccca |
| | | ttggcaccagatacctgactcgtaatctgtaa |
| 109 | Capsid C | gctgccgatggttatcttccagattggctcgaggacaaccttagtgaagg |
| | (trans) | aattcgcgagtggtgggctttgaaacctggagcccctcaacccaaggcaa |
| | wtAAV9 | atcaacaacatcaagacaacgctcgaggtcttgtgcttccgggttacaaa |
| | mutated | taccttggacccggcaacggactcgacaagggggagccggtcaacgcagc |
| | VP1 start | agacgcggcggccctcgagcacgacaaggcctacgaccagcagctcaagg |
| | codon | ccggagacaacccgtacctcaagtacaaccacgccgacgccgagttccag |
| | | gagcggctcaaagaagatacgtcttttgggggcaacctcgggcgagcagt |
| | | cttccaggccaaaaagaggcttcttgaacctcttggtctggttgaggaag |
| | | cggctaagacggctcctggaaagaagaggcctgtagagcagtctcctcag |
| | | gaaccggactcctccgcgggtattggcaaatcgggtgcacagcccgctaa |
| | | aaagagactcaatttcggtcagactggcgacacagagtcagtcccagacc |
| | | ctcaaccaatcggagaacctcccgcagccccctcaggtgtgggatctctt |
| | | acaatggcttcaggtggtggcgcaccagtggcagacaataacgaaggtgc |
| | | cgatggagtgggtagttcctcgggaaattggcattgcgattcccaatggc |
| | | tgggggacagagtcatcaccaccagcacccgaacctgggccctgcccacc |
| | | tacaacaatcacctctacaagcaaatctccaacagcacatctggaggatc |
| | | ttcaaatgacaacgcctacttcggctacagcaccccctgggggtattttg |
| | | acttcaacagattccactgccacttctcaccacgtgactggcagcgactc |
| | | atcaacaacaactggggattccggcctaagcgactcaacttcaagctctt |
| | | caacattcaggtcaaagaggttacggacaacaatggagtcaagaccatcg |
| | | ccaataaccttaccagcacggtccaggtcttcacggactcagactatcag |
| | | ctcccgtacgtgctcgggtcggctcacgagggctgcctcccgccgttccc |
| | | agcggacgttttcatgattcctcagtacgggtatctgacgcttaatgatg |
| | | gaagccaggccgtgggtcgttcgtccttttactgcctggaatatttcccg |
| | | tcgcaaatgctaagaacgggtaacaacttccagttcagctacgagtttga |
| | | gaacgtacctttccatagcagctacgctcacagccaaagcctggaccgac |
| | | taatgaatccactcatcgaccaatacttgtactatctctcaaagactatt |
| | | aacggttctggacagaatcaacaaacgctaaaattcagtgtggccggacc |
| | | cagcaacatggctgtccagggaagaaactacatacctggacccagctacc |
| | | gacaacaacgtgtctcaaccactgtgactcaaaacaacaacagcgaattt |
| | | gcttggcctggagcttcttcttgggctctcaatggacgtaatagcttgat |
| | | gaatcctggacctgctatggccagccacaaagaaggagaggaccgtttct |
| | | ttcctttgtctggatctttaatttttggcaaacaaggaactggaagagac |
| | | aacgtggatgcggacaaagtcatgataaccaacgaagaagaaattaaaac |
| | | tactaacccggtagcaacggagtcctatggacaagtggccacaaaccacc |
| | | agagtgcccaagcacaggcgcagaccggctgggttcaaaaccaaggaata |
| | | cttccgggtatggtttggcaggacagagatgtgtacctgcaaggacccat |
| | | ttgggccaaaattcctcacacggacggcaactttcacccttctccgctga |
| | | tgggagggtttggaatgaagcacccgcctcctcagatcctcatcaaaaac |
| | | acacctgtacctgcggatcctccaacggccttcaacaaggacaagctgaa |
| | | ctctttcatcacccagtattctactggccaagtcagcgtggagatcgagt |
| | | gggagctgcagaaggaaaacagcaagcgctggaacccggagatccagtac |
| | | acttccaactattacaagtctaataatgttgaatttgctgttaatactga |
| | | aggtgtatatagtgaaccccgccccattggcaccagatacctgactcgta |
| | | atctgtaa |
| 110 | Capsid C | atgccggggttttacgagattgtgattaaggtccccagcgaccttgacga |
| | (trans) | gcatctgcccggcatttctgacagctttgtgaactggggtgccgagaagg |
| | Rep2/Cap | aatgggagttgccgccagattctgacatggatctgaatctgattgagcag |
| | wtAAV9 | gcacccctgaccgtggccgagaagctgcagcgcgactttctgacggaatg |
| | mutated | gcgccgtgtgagtaaggccccggaggctcttttctttgtgcaatttgaga |
| | VP1 start | agggagagagctacttccacatgcacgtgctcgtggaaaccaccggggtg |
| | codon | aaatccatggttttgggacgtttcctgagtcagattcgcgaaaaactgat |
| | | tcagagaatttaccgcgggatcgagccgactttgccaaactggttcgcgg |
| | | tcacaaagaccagaaatggcgccggaggcgggaacaaggtggtggatgag |
| | | tgctacatccccaattacttgctccccaaaacccagcctgagctccagtg |
| | | ggcgtggactaatatggaacagtatttaagcgcctgtttgaatctcacgg |
| | | agcgtaaacggttggtggcgcagcatctgacgcacgtgtcgcagacgcag |
| | | gagcagaacaaagagaatcagaatcccaattctgatgcgccggtgatcag |
| | | atcaaaaacttcagccaggtacatggagctggtcgggtggctcgtggaca |
| | | aggggattacctcggagaagcagtggatccaggaggaccaggcctcatac |
| | | atctccttcaatgcggcctccaactcgcggtcccaaatcaaggctgcctt |
| | | ggacaatgcgggaaagattatgagcctgactaaaaccgcccccgactacc |
| | | tggtgggccagcagcccgtggaggacatttccagcaatcggatttataaa |
| | | attttggaactaaacgggtacgatccccaatatgcggcttccgtctttct |
| | | gggatgggccacgaaaaagttcggcaagaggaacaccatctggctgtttg |
| | | ggcctgcaactaccgggaagaccaacatcgcggaggccatagcccacact |
| | | gtgcccttctacgggtgcgtaaactggaccaatgagaactttcccttcaa |
| | | cgactgtgtcgacaagatggtgatctggtgggaggaggggaagatgaccg |
| | | ccaaggtcgtggagtcggccaaagccattctcggaggaagcaaggtgcgc |
| | | gtggaccagaaatgcaagtcctcggcccagatagacccgactcccgtgat |
| | | cgtcacctccaacaccaacatgtgcgccgtgattgacgggaactcaacga |
| | | ccttcgaacaccagcagccgttgcaagaccggatgttcaaatttgaactc |
| | | acccgccgtctggatcatgactttgggaaggtcaccaagcaggaagtcaa |
| | | agactttttccggtgggcaaaggatcacgtggttgaggtggagcatgaat |
| | | tctacgtcaaaaagggtggagccaagaaaagacccgcccccagtgacgca |
| | | gatataagtgagcccaaacgggtgcgcgagtcagttgcgcagccatcgac |
| | | gtcagacgcggaagcttcgatcaactacgcagacaggtaccaaaacaaat |
| | | gttctcgtcacgtgggcatgaatctgatgctgtttccctgcagacaatgc |
| | | gagagaatgaatcagaattcaaatatctgcttcactcacggacagaaaga |
| | | ctgtttagagtgctttcccgtgtcagaatctcaacccgtttctgtcgtca |
| | | aaaaggcgtatcagaaactgtgctacattcatcatatcatgggaaaggtg |
| | | ccagacgcttgcactgcctgcgatctggtcaatgtggatttggatgactg |
| | | catctttgaacaataaatgacttaaaccaggttaggctgccgatggttat |
| | | cttccagattggctcgaggacaaccttagtgaaggaattcgcgagtggtg |
| | | ggctttgaaacctggagcccctcaacccaaggcaaatcaacaacatcaag |
| | | acaacgctcgaggtcttgtgcttccgggttacaaataccttggacccggc |
| | | aacggactcgacaagggggagccggtcaacgcagcagacgcggcggccct |
| | | cgagcacgacaaggcctacgaccagcagctcaaggccggagacaacccgt |
| | | acctcaagtacaaccacgccgacgccgagttccaggagcggctcaaagaa |
| | | gatacgtcttttgggggcaacctcgggcgagcagtcttccaggccaaaaa |
| | | gaggcttcttgaacctcttggtctggttgaggaagcggctaagacggctc |
| | | ctggaaagaagaggcctgtagagcagtctcctcaggaaccggactcctcc |
| | | gcgggtattggcaaatcgggtgcacagcccgctaaaaagagactcaattt |
| | | cggtcagactggcgacacagagtcagtcccagaccctcaaccaatcggag |
| | | aacctcccgcagccccctcaggtgtgggatctcttacaatggcttcaggt |
| | | ggtggcgcaccagtggcagacaataacgaaggtgccgatggagtgggtag |
| | | ttcctcgggaaattggcattgcgattcccaatggctgggggacagagtca |
| | | tcaccaccagcacccgaacctgggccctgcccacctacaacaatcacctc |
| | | tacaagcaaatctccaacagcacatctggaggatcttcaaatgacaacgc |
| | | ctacttcggctacagcaccccctgggggtattttgacttcaacagattcc |
| | | actgccacttctcaccacgtgactggcagcgactcatcaacaacaactgg |
| | | ggattccggcctaagcgactcaacttcaagctcttcaacattcaggtcaa |
| | | agaggttacggacaacaatggagtcaagaccatcgccaataaccttacca |
| | | gcacggtccaggtcttcacggactcagactatcagctcccgtacgtgctc |
| | | gggtcggctcacgagggctgcctcccgccgttcccagcggacgttttcat |
| | | gattcctcagtacgggtatctgacgcttaatgatggaagccaggccgtgg |
| | | gtcgttcgtccttttactgcctggaatatttcccgtcgcaaatgctaaga |
| | | acgggtaacaacttccagttcagctacgagtttgagaacgtacctttcca |
| | | tagcagctacgctcacagccaaagcctggaccgactaatgaatccactca |
| | | tcgaccaatacttgtactatctctcaaagactattaacggttctggacag |
| | | aatcaacaaacgctaaaattcagtgtggccggacccagcaacatggctgt |
| | | ccagggaagaaactacatacctggacccagctaccgacaacaacgtgtct |
| | | caaccactgtgactcaaaacaacaacagcgaatttgcttggcctggagct |
| | | tcttcttgggctctcaatggacgtaatagcttgatgaatcctggacctgc |
| | | tatggccagccacaaagaaggagaggaccgtttctttcctttgtctggat |
| | | ctttaatttttggcaaacaaggaactggaagagacaacgtggatgcggac |
| | | aaagtcatgataaccaacgaagaagaaattaaaactactaacccggtagc |
| | | aacggagtcctatggacaagtggccacaaaccaccagagtgcccaagcac |
| | | aggcgcagaccggctgggttcaaaaccaaggaatacttccgggtatggtt |
| | | tggcaggacagagatgtgtacctgcaaggacccatttgggccaaaattcc |
| | | tcacacggacggcaactttcacccttctccgctgatgggagggtttggaa |
| | | tgaagcacccgcctcctcagatcctcatcaaaaacacacctgtacctgcg |
| | | gatcctccaacggccttcaacaaggacaagctgaactctttcatcaccca |
| | | gtattctactggccaagtcagcgtggagatcgagtgggagctgcagaagg |
| | | aaaacagcaagcgctggaacccggagatccagtacacttccaactattac |
| | | aagtctaataatgttgaatttgctgttaatactgaaggtgtatatagtga |
| | | accccgccccattggcaccagatacctgactcgtaatctgtaa |
| 111 | Capsid E or | gctgccgatggttatcttccagattggctcgaggacaaccttagtgaagg |
| | Capsid M | aattcgcgagtggtgggctttgaaacctggagcccctcaacccaaggcaa |
| | or Capsid N | atcaacaacatcaagacaacgctcgaggtcttgtgcttccgggttacaaa |
| | (trans) | taccttggacccggcaacggactcgacaagggggagccggtcaacgcagc |
| | Cap9 | agacgcggcggccctcgagcacgacaaggcctacgaccagcagctcaagg |
| | | ccggagacaacccgtacctcaagtacaaccacgccgacgccgagttccag |
| | | gagcggctcaaagaagatacgtcttttgggggcaacctcgggcgagcagt |
| | | cttccaggccaaaaagaggcttcttgaacctcttggtctggttgaggaag |
| | | cggctaagacggctcctggaaagaagaggcctgtagagcagtctcctcag |
| | | gaaccggactcctccgcgggtattggcaaatcgggtgcacagcccgctaa |
| | | aaagagactcaatttcggtcagactggcgacacagagtcagtcccagacc |
| | | ctcaaccaatcggagaacctcccgcagccccctcaggtgtgggatctctt |
| | | acaatggcttcaggtggtggcgcaccagtggcagacaataacgaaggtgc |
| | | cgatggagtgggtagttcctcgggaaattggcattgcgattcccaatggc |
| | | tgggggacagagtcatcaccaccagcacccgaacctgggccctgcccacc |
| | | tacaacaatcacctctacaagcaaatctccaacagcacatctggaggatc |
| | | ttcaaatgacaacgcctacttcggctacagcaccccctgggggtattttg |
| | | acttcaacagattccactgccacttctcaccacgtgactggcagcgactc |
| | | atcaacaacaactggggattccggcctaagcgactcaacttcaagctctt |
| | | caacattcaggtcaaagaggttacggacaacaatggagtcaagaccatcg |
| | | ccaataaccttaccagcacggtccaggtcttcacggactcagactatcag |
| | | ctcccgtacgtgctcgggtcggctcacgagggctgcctcccgccgttccc |
| | | agcggacgttttcatgattcctcagtacgggtatctgacgcttaatgatg |
| | | gaagccaggccgtgggtcgttcgtccttttactgcctggaatatttcccg |
| | | tcgcaaatgctaagaacgggtaacaacttccagttcagctacgagtttga |
| | | gaacgtacctttccatagcagctacgctcacagccaaagcctggaccgac |
| | | taatgaatccactcatcgaccaatacttgtactatctctcaaagactatt |
| | | aacggttctggacagaatcaacaaacgctaaaattcagtgtggccggacc |
| | | cagcaacatggctgtccagggaagaaactacatacctggacccagctacc |
| | | gacaacaacgtgtctcaaccactgtgactcaaaacaacaacagcgaattt |
| | | gcttggcctggagcttcttcttgggctctcaatggacgtaatagcttgat |
| | | gaatcctggacctgctatggccagccacaaagaaggagaggaccgtttct |
| | | ttcctttgtctggatctttaatttttggcaaacaaggaactggaagagac |
| | | aacgtggatgcggacaaagtcatgataaccaacgaagaagaaattaaaac |
| | | tactaacccggtagcaacggagtcctatggacaagtggccacaaaccacc |
| | | agagtgcccaagcacaggcgcagaccggctgggttcaaaaccaaggaata |
| | | cttccgggtatggtttggcaggacagagatgtgtacctgcaaggacccat |
| | | ttgggccaaaattcctcacacggacggcaactttcacccttctccgctga |
| | | tgggagggtttggaatgaagcacccgcctcctcagatcctcatcaaaaac |
| | | acacctgtacctgcggatcctccaacggccttcaacaaggacaagctgaa |
| | | ctctttcatcacccagtattctactggccaagtcagcgtggagatcgagt |
| | | gggagctgcagaaggaaaacagcaagcgctggaacccggagatccagtac |
| | | acttccaactattacaagtctaataatgttgaatttgctgttaatactga |
| | | aggtgtatatagtgaaccccgccccattggcaccagatacctgactcgta |
| | | atctgtaa |
| 112 | Capsid E or | atgccggggttttacgagattgtgattaaggtccccagcgaccttgacga |
| | Capsid M | gcatctgcccggcatttctgacagctttgtgaactgggtggccgagaagg |
| | or Capsid N | aatgggagttgccgccagattctgacatggatctgaatctgattgagcag |
| | (trans) | gcacccctgaccgtggccgagaagctgcagcgcgactttctgacggaatg |
| | Rep2/Cap9 | gcgccgtgtgagtaaggccccggaggctcttttctttgtgcaatttgaga |
| | | agggagagagctacttccacatgcacgtgctcgtggaaaccaccggggtg |
| | | aaatccatggttttgggacgtttcctgagtcagattcgcgaaaaactgat |
| | | tcagagaatttaccgcgggatcgagccgactttgccaaactggttcgcgg |
| | | tcacaaagaccagaaatggcgccggaggcgggaacaaggtggtggatgag |
| | | tgctacatccccaattacttgctccccaaaacccagcctgagctccagtg |
| | | ggcgtggactaatatggaacagtatttaagcgcctgtttgaatctcacgg |
| | | agcgtaaacggttggtggcgcagcatctgacgcacgtgtcgcagacgcag |
| | | gagcagaacaaagagaatcagaatcccaattctgatgcgccggtgatcag |
| | | atcaaaaacttcagccaggtacatggagctggtcgggtggctcgtggaca |
| | | aggggattacctcggagaagcagtggatccaggaggaccaggcctcatac |
| | | atctccttcaatgcggcctccaactcgcggtcccaaatcaaggctgcctt |
| | | ggacaatgcgggaaagattatgagcctgactaaaaccgcccccgactacc |
| | | tggtgggccagcagcccgtggaggacatttccagcaatcggatttataaa |
| | | attttggaactaaacgggtacgatccccaatatgcggcttccgtctttct |
| | | gggatgggccacgaaaaagttcggcaagaggaacaccatctggctgtttg |
| | | ggcctgcaactaccgggaagaccaacatcgcggaggccatagcccacact |
| | | gtgcccttctacgggtgcgtaaactggaccaatgagaactttcccttcaa |
| | | cgactgtgtcgacaagatggtgatctggtgggaggaggggaagatgaccg |
| | | ccaaggtcgtggagtcggccaaagccattctcggaggaagcaaggtgcgc |
| | | gtggaccagaaatgcaagtcctcggcccagatagacccgactcccgtgat |
| | | cgtcacctccaacaccaacatgtgcgccgtgattgacgggaactcaacga |
| | | ccttcgaacaccagcagccgttgcaagaccggatgttcaaatttgaactc |
| | | acccgccgtctggatcatgactttgggaaggtcaccaagcaggaagtcaa |
| | | agactttttccggtgggcaaaggatcacgtggttgaggtggagcatgaat |
| | | tctacgtcaaaaagggtggagccaagaaaagacccgcccccagtgacgca |
| | | gatataagtgagcccaaacgggtgcgcgagtcagttgcgcagccatcgac |
| | | gtcagacgcggaagcttcgatcaactacgcagacaggtaccaaaacaaat |
| | | gttctcgtcacgtgggcatgaatctgatgctgtttccctgcagacaatgc |
| | | gagagaatgaatcagaattcaaatatctgcttcactcacggacagaaaga |
| | | ctgtttagagtgctttcccgtgtcagaatctcaacccgtttctgtcgtca |
| | | aaaaggcgtatcagaaactgtgctacattcatcatatcatgggaaaggtg |
| | | ccagacgcttgcactgcctgcgatctggtcaatgtggatttggatgactg |
| | | catctttgaacaataaatgacttaaaccaggttaggctgccgatggttat |
| | | cttccagattggctcgaggacaaccttagtgaaggaattcgcgagtggtg |
| | | ggctttgaaacctggagcccctcaacccaaggcaaatcaacaacatcaag |
| | | acaacgctcgaggtcttgtgcttccgggttacaaataccttggacccggc |
| | | aacggactcgacaagggggagccggtcaacgcagcagacgcggcggccct |
| | | cgagcacgacaaggcctacgaccagcagctcaaggccggagacaacccgt |
| | | acctcaagtacaaccacgccgacgccgagttccaggagcggctcaaagaa |
| | | gatacgtcttttgggggcaacctcgggcgagcagtcttccaggccaaaaa |
| | | gaggcttcttgaacctcttggtctggttgaggaagcggctaagacggctc |
| | | ctggaaagaagaggcctgtagagcagtctcctcaggaaccggactcctcc |
| | | gcgggtattggcaaatcgggtgcacagcccgctaaaaagagactcaattt |
| | | cggtcagactggcgacacagagtcagtcccagaccctcaaccaatcggag |
| | | aacctcccgcagccccctcaggtgtgggatctcttacaatggcttcaggt |
| | | ggtggcgcaccagtggcagacaataacgaaggtgccgatggagtgggtag |
| | | ttcctcgggaaattggcattgcgattcccaatggctgggggacagagtca |
| | | tcaccaccagcacccgaacctgggccctgcccacctacaacaatcacctc |
| | | tacaagcaaatctccaacagcacatctggaggatcttcaaatgacaacgc |
| | | ctacttcggctacagcaccccctgggggtattttgacttcaacagattcc |
| | | actgccacttctcaccacgtgactggcagcgactcatcaacaacaactgg |
| | | ggattccggcctaagcgactcaacttcaagctcttcaacattcaggtcaa |
| | | agaggttacggacaacaatggagtcaagaccatcgccaataaccttacca |
| | | gcacggtccaggtcttcacggactcagactatcagctcccgtacgtgctc |
| | | gggtcggctcacgagggctgcctcccgccgttcccagcggacgttttcat |
| | | gattcctcagtacgggtatctgacgcttaatgatggaagccaggccgtgg |
| | | gtcgttcgtccttttactgcctggaatatttcccgtcgcaaatgctaaga |
| | | acgggtaacaacttccagttcagctacgagtttgagaacgtacctttcca |
| | | tagcagctacgctcacagccaaagcctggaccgactaatgaatccactca |
| | | tcgaccaatacttgtactatctctcaaagactattaacggttctggacag |
| | | aatcaacaaacgctaaaattcagtgtggccggacccagcaacatggctgt |
| | | ccagggaagaaactacatacctggacccagctaccgacaacaacgtgtct |
| | | caaccactgtgactcaaaacaacaacagcgaatttgcttggcctggagct |
| | | tcttcttgggctctcaatggacgtaatagcttgatgaatcctggacctgc |
| | | tatggccagccacaaagaaggagaggaccgtttctttcctttgtctggat |
| | | ctttaatttttggcaaacaaggaactggaagagacaacgtggatgcggac |
| | | aaagtcatgataaccaacgaagaagaaattaaaactactaacccggtagc |
| | | aacggagtcctatggacaagtggccacaaaccaccagagtgcccaagcac |
| | | aggcgcagaccggctgggttcaaaaccaaggaatacttccgggtatggtt |
| | | tggcaggacagagatgtgtacctgcaaggacccatttgggccaaaattcc |
| | | tcacacggacggcaactttcacccttctccgctgatgggagggtttggaa |
| | | tgaagcacccgcctcctcagatcctcatcaaaaacacacctgtacctgcg |
| | | gatcctccaacggccttcaacaaggacaagctgaactctttcatcaccca |
| | | gtattctactggccaagtcagcgtggagatcgagtgggagctgcagaagg |
| | | aaaacagcaagcgctggaacccggagatccagtacacttccaactattac |
| | | aagtctaataatgttgaatttgctgttaatactgaaggtgtatatagtga |
| | | accccgccccattggcaccagatacctgactcgtaatctgtaa |
| 116 | Rep 2 | atgccggggttttacgagattgtgattaaggtccccagcgaccttgacga |
| | sequence | gcatctgcccggcatttctgacagctttgtgaactgggtggccgagaagg |
| | | aatgggagttgccgccagattctgacatggatctgaatctgattgagcag |
| | | gcacccctgaccgtggccgagaagctgcagcgcgactttctgacggaatg |
| | | gcgccgtgtgagtaaggccccggaggctcttttctttgtgcaatttgaga |
| | | agggagagagctacttccacatgcacgtgctcgtggaaaccaccggggtg |
| | | aaatccatggttttgggacgtttcctgagtcagattcgcgaaaaactgat |
| | | tcagagaatttaccgcgggatcgagccgactttgccaaactggttcgcgg |
| | | tcacaaagaccagaaatggcgccggaggcgggaacaaggtggtggatgag |
| | | tgctacatccccaattacttgctccccaaaacccagcctgagctccagtg |
| | | ggcgtggactaatatggaacagtatttaagcgcctgtttgaatctcacgg |
| | | agcgtaaacggttggtggcgcagcatctgacgcacgtgtcgcagacgcag |
| | | gagcagaacaaagagaatcagaatcccaattctgatgcgccggtgatcag |
| | | atcaaaaacttcagccaggtacatggagctggtcgggtggctcgtggaca |
| | | aggggattacctcggagaagcagtggatccaggaggaccaggcctcatac |
| | | atctccttcaatgcggcctccaactcgcggtcccaaatcaaggctgcctt |
| | | ggacaatgcgggaaagattatgagcctgactaaaaccgcccccgactacc |
| | | tggtgggccagcagcccgtggaggacatttccagcaatcggatttataaa |
| | | attttggaactaaacgggtacgatccccaatatgcggcttccgtctttct |
| | | gggatgggccacgaaaaagttcggcaagaggaacaccatctggctgtttg |
| | | ggcctgcaactaccgggaagaccaacatcgcggaggccatagcccacact |
| | | gtgcccttctacgggtgcgtaaactggaccaatgagaactttcccttcaa |
| | | cgactgtgtcgacaagatggtgatctggtgggaggaggggaagatgaccg |
| | | ccaaggtcgtggagtcggccaaagccattctcggaggaagcaaggtgcgc |
| | | gtggaccagaaatgcaagtcctcggcccagatagacccgactcccgtgat |
| | | cgtcacctccaacaccaacatgtgcgccgtgattgacgggaactcaacga |
| | | ccttcgaacaccagcagccgttgcaagaccggatgttcaaatttgaactc |
| | | acccgccgtctggatcatgactttgggaaggtcaccaagcaggaagtcaa |
| | | agactttttccggtgggcaaaggatcacgtggttgaggtggagcatgaat |
| | | tctacgtcaaaaagggtggagccaagaaaagacccgcccccagtgacgca |
| | | gatataagtgagcccaaacgggtgcgcgagtcagttgcgcagccatcgac |
| | | gtcagacgcggaagcttcgatcaactacgcagacaggtaccaaaacaaat |
| | | gttctcgtcacgtgggcatgaatctgatgctgtttccctgcagacaatgc |
| | | gagagaatgaatcagaattcaaatatctgcttcactcacggacagaaaga |
| | | ctgtttagagtgctttcccgtgtcagaatctcaacccgtttctgtcgtca |
| | | aaaaggcgtatcagaaactgtgctacattcatcatatcatgggaaaggtg |
| | | ccagacgcttgcactgcctgcgatctggtcaatgtggatttggatgactg |
| | | catctttgaacaa |
| 120 | Capsid L | ctagtcgacattgattattgactagttattaatagtaatcaattacgggg |
| | VP2.VR4 | tcattagttcatagcccatatatggagttccgcgttacataacttacggt |
| | Secondary | aaatggcccgcctggctgaccgcccaacgacccccgcccattgacgtcaa |
| | plasmid | taatgacgtatgttcccatagtaacgccaatagggactttccattgacgt |
| | cassette | caatgggtggagtatttacggtaaactgcccacttggcagtacatcaagt |
| | with CAG | gtatcatatgccaagtacgccccctattgacgtcaatgacggtaaatggc |
| | promoter | ccgcctggcattatgcccagtacatgaccttatgggactttcctacttgg |
| | (including | cagtacatctacgtattagtcatcgctattaccatggtcgaggtgagccc |
| | linkers | cacgttctgcttcactctccccatctcccccccctccccacccccaattt |
| | flanking | tgtatttatttattttttaattattttgtgcagcgatgggggcggggggg |
| | *DARPin | gggggggggcgcgcgccaggcggggcggggcggggcgaggggcggggcgg |
| | insertion | ggcgaggcggagaggtgcggcggcagccaatcagagcggcgcgctccgaa |
| | site) | agtttccttttatggcgaggcggcggcggcggcggccctataaaaagcga |
| | | agcgcgcggcgggcgggagtcgctgcgcgctgccttcgccccgtgccccg |
| | | ctccgccgccgcctcgcgccgcccgccccggctctgactgaccgcgttac |
| | | tcccacaggtgagcgggcgggacggcccttctcctccgggctgtaattag |
| | | cgcttggtttaatgacggcttgtttcttttctgtggctgcgtgaaagcct |
| | | tgaggggctccgggagggccctttgtgcggggggagcggctcggggggtg |
| | | cgtgcgtgtgtgtgtgcgtggggagcgccgcgtgcggctccgcgctgccc |
| | | ggcggctgtgagcgctgcgggcgcggcgcggggctttgtgcgctccgcag |
| | | tgtgcgcgaggggagcgcggccgggggcggtgccccgcggtgcggggggg |
| | | gctgcgaggggaacaaaggctgcgtgcggggtgtgtgcgtgggggggtga |
| | | gcagggggtgtgggcgcgtcggtcgggctgcaaccccccctgcacccccc |
| | | tccccgagttgctgagcacggcccggcttcgggtgcggggctccgtacgg |
| | | ggcgtggcgcggggctcgccgtgccgggcggggggtggcggcaggtgggg |
| | | gtgccgggcggggcggggccgcctcgggccggggagggctcgggggaggg |
| | | gcgcggcggcccccggagcgccggcggctgtcgaggcgcggcgagccgca |
| | | gccattgccttttatggtaatcgtgcgagagggcgcagggacttcctttg |
| | | tcccaaatctgtgcggagccgaaatctgggaggcgccgccgcaccccctc |
| | | tagcgggcgcggggcgaagcggtgcggcgccggcaggaaggaaatgggcg |
| | | gggagggccttcgtgcgtcgccgcgccgccgtccccttctccctctccag |
| | | cctcggggctgtccgcggggggacggctgccttcgggggggacggggcag |
| | | ggcggggttcggcttctggcgtgtgaccggcggctctagagcctctgcta |
| | | accatgttcatgccttcttctttttcctacagctcctgggcaacgtgctg |
| | | gttattgtgctgtctcatcattttggcaaagccaccatggctcctggaaa |
| | | gaagaggcctgtagagcagtctcctcaggaaccggactcctccgcgggta |
| | | ttggcaaatcgggtgcacagcccgctaaaaagagactcaatttcggtcag |
| | | actggcgacacagagtcagtcccagaccctcaaccaatcggagaacctcc |
| | | cgcagccccctcaggtgtgggatctcttacaatggcttcaggtggtggcg |
| | | caccagtggcagacaataacgaaggtgccgatggagtgggtagttcctcg |
| | | ggaaattggcattgcgattcccaatggctgggggacagagtcatcaccac |
| | | cagcacccgaacctgggccctgcccacctacaacaatcacctctacaagc |
| | | aaatctccaacagcacatctggaggatcttcaaatgacaacgcctacttc |
| | | ggctacagcaccccctgggggtattttgacttcaacagattccactgcca |
| | | cttctcaccacgtgactggcagcgactcatcaacaacaactggggattcc |
| | | ggcctaagcgactcaacttcaagctcttcaacattcaggtcaaagaggtt |
| | | acggacaacaatggagtcaagaccatcgccaataaccttaccagcacggt |
| | | ccaggtcttcacggactcagactatcagctcccgtacgtgctcgggtcgg |
| | | ctcacgagggctgcctcccgccgttcccagcggacgttttcatgattcct |
| | | cagtacgggtatctgacgcttaatgatggaagccaggccgtgggtcgttc |
| | | gtccttttactgcctggaatatttcccgtcgcaaatgctaagaacgggta |
| | | acaacttccagttcagctacgagtttgagaacgtacctttccatagcagc |
| | | tacgctcacagccaaagcctggaccgactaatgaatccactcatcgacca |
| | | atacttgtactatctctcaaagactattaacggctctggcggcggcggca |
| | | gcggcggaggcggctccggcggaggcggcagcggcggaggaggttctgga |
| | | agc* |
| | | ggctctggcggcggcggcagcggcggaggcggctccggcggaggcggcag |
| | | cggcggaggaggttctggaagccaaacgctaaaattcagtgtggccggac |
| | | ccagcaacatggctgtccagggaagaaactacatacctggacccagctac |
| | | cgacaacaacgtgtctcaaccactgtgactcaaaacaacaacagcgaatt |
| | | tgcttggcctggagcttcttcttgggctctcaatggacgtaatagcttga |
| | | tgaatcctggacctgctatggccagccacaaagaaggagaggaccgtttc |
| | | tttcctttgtctggatctttaatttttggcaaacaaggaactggaagaga |
| | | caacgtggatgcggacaaagtcatgataaccaacgaagaagaaattaaaa |
| | | ctactaacccggtagcaacggagtcctatggacaagtggccacaaaccac |
| | | cagagtgcccaagcacaggcgcagaccggctgggttcaaaaccaaggaat |
| | | acttccgggtatggtttggcaggacagagatgtgtacctgcaaggaccca |
| | | tttgggccaaaattcctcacacggacggcaactttcacccttctccgctg |
| | | atgggagggtttggaatgaagcacccgcctcctcagatcctcatcaaaaa |
| | | cacacctgtacctgcggatcctccaacggccttcaacaaggacaagctga |
| | | actctttcatcacccagtattctactggccaagtcagcgtggagatcgag |
| | | tgggagctgcagaaggaaaacagcaagcgctggaacccggagatccagta |
| | | cacttccaactattacaagtctaataatgttgaatttgctgttaatactg |
| | | aaggtgtatatagtgaaccccgccccattggcaccagatacctgactcgt |
| | | aatctgtaa |

TABLE 9

| SEQ | | |
|---|---|---|
| ID | Brief | |
| NO | Description | Sequence |
| 59 | AAV1 | MAADGYLPDWLEDNLSEGIREWWDLKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVEQAKKRVL |
| | | EPLGLVEEGAKTAPGKKRPVEQSPQEPDSSSGIGKTGQQPAKKRLNFGQTGDSESVPDPQPLG |
| | | EPPATPAAVGPTTMASGGGAPMADNNEGADGVGNASGNWECDSTWLGDVITTSTRTWALPTYN |
| | | NHLYKQISSASTGASNDNHYFGYSTPWGYFDFNRFHCHFSPRDWQRLINNNWGFRPKRLNFKL |
| | | FNIQVKEVTTNDGVTTIANNLTSTVQVFSDSEYQLPYVLGSAHQGCLPPFPADVEMIPQYGYL |
| | | TINNGSQAVGRSSFYCLEYFPSQMLRTGNNFTFSYTFEEVPFHSSYAHSQSLDRLMNPLIDQY |
| | | LYYLNRTQNQSGSAQNKDLLFSRGSPAGMSVQPKNWLPGPCYRQQRVSKTKIDNNNSNFTWTG |
| | | ASKYNLNGRESIINPGTAMASHKDDEDKFFPMSGVMIFGKESAGASNTALDNVMITDEEEIKA |
| | | TNPVATERFGTVAVNFQSSS#TDPATGDVHAMGALPGMVWQDRDVYLQGPIWAKIPHTDGHFH |
| | | PSPLMGGFGLKNPPPQILIKNTPVPANPPAEFSATKEASFITQYSTGQVSVEIEWELQKENSK |
| | | RWNPEVQYTSNYAKSANVDFTVDNNGLYTEPRPIGTRYLTRPL |
| 60 | AAV2 | MAADGYLPDWLEDTLSEGIRQWWKLKPGPPPPKPAERHKDDSRGLVLPGYKYLGPENGLDKGE |
| | | PVNEADAAALEHDKAYDRQLDSGDNPYLKYNHADAEFQERLKEDTSFGGNLGRAVEQAKKRVL |
| | | EPLGLVEEPVKTAPGKKRPVEHSPVEPDSSSGTGKAGQQPARKRLNFGQTGDADSVPDPQPLG |
| | | QPPAAPSGLGTNTMATGSGAPMADNNEGADGVGNSSGNWECDSTWMGDRVITTSTRTWALPTY |
| | | NNHLYKQISSQSGASNDNHYFGYSTPWGYFDENRFHCHFSPRDWQRLINNNWGFRPKRLNFKL |
| | | ENIQVKEVTQNDGITTIANNLTSTVQVETDSEYQLPYVLGSAHQGCLPPFPADVEMVPQYGYL |
| | | TLNNGSQAVGRSSFYCLEYFPSQMLRTGNNFTFSYTFEDVPFHSSYAHSQSLDRLMNPLIDQY |
| | | LYYLSRTNTPSGTTTQSRLQFSQAGASDIRDQSRNWLPGPCYRQCRVSKTSADNNNSEYSWTG |
| | | ATKYHINGRDSLVNPGPAMASHKDDEEKFFPQSGVLIFGKQGSEKTNVDIEKVMITDEEEIRT |
| | | TNPVATEQYGSVSTNLQRGN#RQAATADVNTQGVLPGMVWQDRDVYLQGPIWAKIPHTDGHFH |
| | | PSPLMGGFGLKHPPPQILIKNTPVPANPSTTFSAAKFASFITQYSTGQVSVEIEWELQKENSK |
| | | RWNPEIQYTSNYNKSVNVDFTVDINGVYSEPRPIGTRYLTRNL |
| 61 | AAV3 | MAADGYLPDWLEDNLSEGIREWWALKPGVPQPKANQQHQDNRRGLVLPGYKYLGPGNGLDKGE |
| | | PVNEADAAALEHDKAYDQQLKAGDNPYLKYNHADAEFQERLQEDTSFGGNLGRAVEQAKKRIL |
| | | EPLGLVEEAAKTAPGKKGAVDQSPQEPDSSSGVGKSGKQPARKRLNFGQTGDSESVPDPQPLG |
| | | EPPAAPTSLGSNTMASGGGAPMADNNEGADGVGNSSGNWECDSQWLGDRVITTSTRTWALPTY |
| | | NNHLYKQISSQSGASNDNHYFGYSTPWGYFDENRFHCHESPRDWQRLINNNWGFRPKKLSFKL |
| | | FNIQVRGVTQNDGTTTIANNLTSTVQVFTDSEYQLPYVLGSAHQGCLPPFPADVEMVPQYGYL |
| | | TINNGSQAVGRSSFYCLEYFPSQMLRTGNNFQFSYTFEDVPFHSSYAHSQSLDRLMNPLIDQY |
| | | LYYLNRTQGTTSGTTNQSRLLFSQAGPQSMSLQARNWLPGPCYRCQRLSKTANDNNNSNFPWT |
| | | AASKYHLNGRDSLVNPGPAMASHKDDEEKFFPKHGNLIFGKEGTTASNAELDNVMITDEEEIR |
| | | TTNPVATEQYGTVANNLQSSN#TAPTTGTVNHQGALPGMVWQDRDVYLQGPIWAKIPHTDGHF |
| | | HPSPLMGGFGLKHPPPQIMIKNTPVPANPPTTFSPAKFASFITQYSTGQVSVEIEWELQKENS |
| | | KRWNPEIQYTSNYNKSVNVDFTVDTNGVYSEPRPIGTRYLTRNL |
| 62 | AAV4 | MTDGYLPDWLEDNLSEGVREWWALQPGAPKPKANQQHQDNARGLVLPGYKYLGPGNGLDKGEP |
| | | VNAADAAALEHDKAYDQQLKAGDNPYLKYNHADAEFQQRLQGDTSFGGNLGRAVFQAKKRVLE |
| | | PLGLVEQAGETAPGKKRPLIESPQQPDSSTGIGKKGKQPAKKKLVFEDETGAGDGPPEGSTSG |
| | | AMSDDSEMRAAAGGAAVEGGQGADGVGNASGDWHCDSTWSEGHVTTTSTRTWVLPTYNNHLYK |
| | | RIGSLQSNTYNGFSTPWGYFDFNRFHCHFSPRDWQRTINNNWGMRPKAMRVKTFNTQVKEVTT |
| | | SNGETTVANNLTSTVQIFADSSYELPYVMDAGQEGSLPPFPNDVFMVPQYGYCGLVTGNTSQQ |
| | | QTDRNAFYCLEYFPSQMLRTENNFEITYSFEKVPFHSMYAHSQSLDRLMNPLIDQYLWGLQST |
| | | TTGTTLNAGTATTNFTKLRPTNFSNFKKNWLPGPSIKQQGFSKTANQNYKIPATGSDSLIKYE |
| | | THSTLDGRWSALTPGPPMATAGPADSKESNSQLIFAGPKQNGNTATVPGTLIFTSEEELAATN |
| | | ATDTDMWGNLPGGDQSNS#NLPTVDRLTALGAVPGMVWQNRDIYYQGPIWAKIPHTDGHFHPS |
| | | PLIGGFGLKHPPPCIFIKNTPVPANPATTESSTPVNSFITQYSTGQVSVQIDWEIQKERSKRW |
| | | NPEVQFTSNYQQNSLLWAPDAAGKYTEPRAIGTRYLTHHL |
| 63 | AAV5 | MSFVDHPPDWLEEVGEGLREFLGLEAGPPKPKPNQQHQDQARGLVLPGYNYLGPGNGLDRGEP |
| | | VNRADEVAREHDISYNEQLEAGDNPYLKYNHADAEFQEKLADDTSFGGNLGKAVFQAKKRVLE |
| | | PFGLVEEGAKTAPTGKRIDDHFPKRKKARTEEDSKPSTSSDAEAGPSGSQQLQIPAQPASSLG |
| | | ADTMSAGGGGPLGDNNQGADGVGNASGDWHCDSTWMGDRVVTKSTRTWVLPSYNNHQYREIKS |
| | | GSVDGSNANAYFGYSTPWGYFDFNRFHSHWSPRDWQRLINNYWGERPRSLRVKIFNIQVKEVT |
| | | VQDSTTTIANNLTSTVQVFTDDDYQLPYVVGNGTEGCLPAFPPQVFTLPQYGYATLNRDNTEN |
| | | PTERSSFFCLEYFPSKMLRTGNNFEFTYNFEEVPFHSSFAPSQNLFKLANPLVDQYLYRFVST |
| | | NNTGGVQFNKNLAGRYANTYKNWFPGPMGRTQGWNLGSGVNRASVSAFATTNRMELEGASYQV |
| | | PPQPNGMTNNLQGSNTYALENTMIFNSQPANPGTTATYLEGNMLITSESETQPVNRVAYNVGG |
| | | QVATNNQSST#TAPATGTYNTQETVPGSVWMERDVYLQGPTWAKTPETGAHFHPSPAMGGFGL |
| | | KHPPPMMLIKNTPVPGNITSFSDVPVSSFITQYSTGQVTVEMEWELKKENSKRWNPEIQYTNN |
| | | YNDPQFVDFAPDSTGEYRTTRPIGTRYLTRPL |
| 64 | AAV6 | MAADGYLPDWLEDNLSEGIREWWDLKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVEQAKKRVL |
| | | EPFGLVEEGAKTAPGKKRPVEQSPQEPDSSSGIGKTGQQPAKKRLNFGQTGDSESVPDPQPLG |
| | | EPPATPAAVGPTTMASGGGAPMADNNEGADGVGNASGNWECDSTWLGDRVITTSTRTWALPTY |
| | | NNHLYKQISSASTGASNDNHYFGYSTPWGYFDFNRFHCHFSPRDWQRLINNNWGFRPKRLNFK |
| | | LFNIQVKEVTTNDGVTTIANNLTSTVQVFSDSEQLPYVLGSAHQGCLPPFPADVEMIPQYGYL |
| | | TINNGSQAVGRSSFYCLEYFPSQMLRTGNNFTFSYTFEDVPFHSSYAHSQSLDRLMNPLIDQY |
| | | LYYLNRTQNQSGSAQNKDLLESRGSPAGMSVQPKNWLPGPCYRQQRVSKTKIDNNNSNFTWTG |
| | | ASKYNINGRESIINPGTAMASHKDDKDKFFPMSGVMIFGKESAGASNTALDNVMITDEEEIKA |
| | | TNPVATERFGTVAVNLQSSS#TDPATGDVHVMGALPGMVWQDRDVYLQGPIWAKIPHTDGHFH |
| | | PSPLMGGFGLKHPPPQILIKNTPVPANPPAEFSATKFASFITQYSTGQVSVEIEWELQKENSK |
| | | RWNPEVQYTSNYAKSANVDFTVDNNGLYTEPRPIGTRYLTRPL |
| 65 | AAV7 | MAADGYLPDWLEDNLSEGIREWWDLKPGAPKPKANQQKQDNGRGLVLPGYKYLGPENGLDKGE |
| | | PVNAADWAALEHDKAYDQQLKAGDNPYLRYNHADAEFQERLQEDTSFGGNLGRAVEQAKKRVL |
| | | EPLGLVEEGAKTAPAKKRPVEPSPQRSPDSSTGIGKKGQQPARKRLNFGQTGDSESVPDPQPL |
| | | GEPPAAPSSVGSGTVAAGGGAPMADNNEGADGVGNASGNWHCDSTWLGDRVITTSTRTWALPT |
| | | YNNHLYKQISSETAGSTNDNTYFGYSTPWGYFDFNRFHCEFSPRLWQRLINNNWGFRPKKLRF |
| | | KLFNIQVKEVTTNDGVTTIANNLTSTIQVFSDSEYQLPYVLGSAHQGCLPPFPADVEMIPQYG |
| | | YLTLNNGSQSVGRSSFYCLEYFPSQMLRTGNNFEFSYSFEDVPFHSSYAHSQSLDRLMNPLID |
| | | QYLYYLARTQSNPGGTAGNRELQFYQGGPSTMAEQAKNWLPGPCFRQQRVSKTLDQNNNSNFA |
| | | NTGATKYHLNGRNSLVNPGVAMATHKDDEDRFFPSSGVLIFGKTGATNKTTLENVLMTNEEEI |
| | | RPTNPVATEEYGTVSSNIQAAN#TAAQTQVVNNQGALPGMVWQNRDVYTQGPTWAKTPHTDGN |
| | | FHPSPLMGGFGLKHPPPQILIKNTPVPADPPTTFNQSKINSFITQYSTGQVSVEIEWELQKEN |
| | | SKRWNPEIQYTSNFEKQTGVDFAVDSQGVYSEPRPIGTRYLTRNL |
| 66 | AAV8 | MAADGYLPDWLEDNLSEGIREWWALKPGAPKPKANQQKQDDGRGLVLPGYKYLGPENGLDKGE |
| | | PVNAADAAALEHDKAYDQQLQAGDNPYLRYNHADAEFQERLQEDTSFGGNLGRAVEQAKKRVL |
| | | EPLGLVEEGAKTAPGKKRPVEPSPQRSPDSSTGIGKKGQQPARKRLNFGQTGDSESVPDPQPL |
| | | GEPPAAPSGVGPNTMAAGGGAPMADNNEGADGVGSSSGNWHCDSTWLGDRVITTSTRTWALPT |
| | | YNNHLYKQISNGTSGGATNDNTYFGYSTPWGYFDFNRFHCHFSPRDWQRLINNNWGFRPKRLS |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFQFTYTFEDVPFHSSYAHSQSLDRLMNPLI |
| | | DQYLYYLSRTQTTGGTANTQTLGFSQGGPNTMANQAKNWLPGPCYRQQRVSTTTGQNNNSNFA |
| | | WTAGTKYHLNGRNSLANPGIAMATHKDDEERFFPSNGILIFGKQNAARDNADYSDVMLTSEEE |
| | | IKTTNPVATEEYGIVADNLQQQN#TAPQIGTVNSQGALPGMVWQNRDVYLQGPIWAKIPHTDG |
| | | NFHPSPLMGGFGLKHPPPQILIKNTPVPADPPTTFNQSKINSFITQYSTGQVSVEIEWELQKE |
| | | NSKRWNPEIQYTSNYYKSTSVDFAVNTEGVYSEPRPIGTRYLTRNL |
| 67 | AAV9 | MAADGYLPDWLEDNLSEGIREWWALKPGAPQPKANQQHQDNARGLVLPGYKYLGPGNGLDKGE |
| | | PVNAADAAALEHDKAYDQQLKAGDNPYLKYNHADAEFQERLKEDTSFGGNLGRAVFQAKKRLL |
| | | EPLGLVEEAAKTAPGKKRPVEQSPQEPDSSAGIGKSGAQPAKKRLNFGQTGDTESVPDPQPIG |
| | | EPPAAPSGVGSLTMASGGGAPVADNNEGADGVGSSSGNWECDSQWLGDRVITTSTRTWALPTY |
| | | NNHLYKQTSNSTSGGSSNDNAYFGYSTPWGYFDFNRFHCEFSPRIWQRTINNNWGFRPKRINF |
| | | KLFNIQVKEVTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSLDRLMNPLID |
| | | QYLYYLSKTINGSGQNQQTLKESVAGPSNMAVQGRNYIPGPSYRQQRVSTTVTQNNNSEFAWP |
| | | GASSWALNGRNSLMNPGPAMASHKEGEDRFFPLSGSLIFGKQGTGRDNVDADKVMITNEEEIK |
| | | TTNPVATESYGQVATNHQSAQ#AQAQTGWVQNQGIL?GMVWQDREVYLQGPIWAKIPHTDGNE |
| | | HPSPLMGGFGMKHPPPQILIKNTPVPADPPTAFNKDKLNSFITQYSTGQVSVEIEWELQKENS |
| | | KRWNPEIQYTSNYYKSNNVEFAVNTEGVYSEPRPIGTRYLTRNL |
| 68 | AAV9e | MAADGYLPDWLEDNLSEGIREWWALKPGAPQPKANQQHQDNARGLVLPGYKYLGPGNGLDKGE |
| | | PVNAADAWALEHDKAYDQQLKAGDNPYLKYNHADAEFQERLKEDTSFGGNLGRAVFQAKKRLL |
| | | EPLGLVEEAAKTAPGKKRPVEQSPQEPDSSAGIGKSGAQPAKKRLNFGQTGDTESVPDPQPIG |
| | | EPPAAPSGVGSLTMASGGGAPVADNNEGADGVGSSSGNWECDSQWLGDRVITTSTRTWALPTY |
| | | NNHLYKQISNSTSGGSSNDNAYFGYSTPWGYFDFNRFHCEFSPREWQRLINNNWGFRPKRLNF |
| | | KLFNIQVKEVTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSLDRLMNPLID |
| | | QYLYYLSKTINGSGQNQQTLKESVAGPSNMAVQGRNYIPGPSYRQQRVSTTVTQNNNSEFAWP |
| | | GASSWALNGRNSLMNPGPAMASHKEGEDRFFPLSGSLIFGKQGTGRDNVDADKVMITNEEEIK |
| | | TTNPVATESYGQVATNHQSDG#AQAQTGWVQNQGTTPGMVWQDREVYLQGPTWAKTPHTDGNF |
| | | HPSPLMGGFGMKHPPPQILIKNTPVPADPPTAFNKDKLNSFITQYSTGQVSVEIEWELQKENS |
| | | KRWNPEIQYTSNYYKSNNVEFAVNTEGVYSEPRPIGTRYLTRNL |
| 69 | AAV.rh10 | MAADGYLPDWLEDNLSEGIREWWDLKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVFQAKKRVL |
| | | EPLGLVEEGAKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRLN |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFEFSYQFEDVPFHSSYAHSQSLDRLMNPLI |
| | | DQYLYYLSRTQSTGGTAGTQQLLFSQAGPNNMSAQAKNWLPGPCYRQQRVSTTLSQNNNSNFA |
| | | WTGATKYHLNGRDSLVNPGVAMATHKDDEERFFPSSGVLMFGKQGAGKDNVDYSSVMLTSEEE |
| | | TKTTNPVATEQYGVVADNLQQQN#AAPIVGAVNSQGALPGMVWQNRDVYLQGPIWAKIPHTDG |
| | | NFHPSPLMGGFGLKHPPPQILIKNTPVPADPPTTFSQAKLASFITQYSTGQVSVEIEWELQKE |
| | | NSKRWNPEIQYTSNYYKSTNVDFAVNTDGTYSEPRPIGTRYLTRNL |
| 70 | AAV.rh20 | MAADGYLPDWLEDNLSEGIREWWDLKPGAPKPKANQQKQDDGRGLVLPGYKYLGPENGLDKGE |
| | | PVNAADAWALEHDKAYDQQLKAGDNPYLRYNHADAEFQERLQEDTSFGGNLGRAVEQAKKRVL |
| | | EPLGLVEEGAKTAPGKKRPVEPSPQRSPDSSTGIGKTGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRLN |
| | | FKLFNIQVKEVTQNEGTKTIANNLTSTIQVFTDSEYQLPYVLGSAHQGCLPPFPADVFMIPQY |
| | | GYLTLNNGSQAVGRSSFYCLEYFPSQMLRTGNNFEFSYQFEDVPFHSSYAHSQSLDRLMNPLI |
| | | DQYLYYLSRTQSTGGTAGTQQLLFSQAGPNNMSAQAKNWLPGPCYRQQRVSTTLSQNNNSNFA |
| | | WTGATKYHLNGRDSLVNPGVAMATHKDDEERFFPSSGVLMFGKQGAGKDNVDYSSVMLTSEEE |
| | | TKTTNPVATEQYGVVADNIQQQN#AAPTVGAVNSQGALPGMVWQNRDVYLQGPTWAKTPHTDG |
| | | NFHPSPLMGGFGLKHPPPQILIKNTPVPADPPTTFSQAKLASFITQYSTGQVSVEIEWELQKE |
| | | NSKRWNPEIQYTSNYYKSTNVDFAVNTEGTYSEPRPIGTRYLTRNL |
| 71 | AAV.hu37 | MAADGYLPDWLEDNLSEGIREWWDLKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVFQAKKRVL |
| | | EPLGLVEEAAKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRESVPDPQPIGEPP |
| | | AGPSGIGSGTMAAGGGAPMADNNEGADGVGSSSGNWHCDSTWLGERVITTSTRTWALPTYNNH |
| | | LYKQISNGTSGGSTNDNTYFGYSTPWGYFDENRFHCHFSPRDWQRLINNNWGFRPKRLSFKLE |
| | | NIQVKEVTQNEGTKTIANNLISTIQVFTDSEYQLPYVLGSAHQGCLPPFPADVEMIPQYGYLT |
| | | LNNGSQAVGRSSFYCLEYFPSQMLRTGNNFEFSYTFEDVPFHSSYAHSQSLDRLMNPLIDQYL |
| | | YYLSRTQSTGGTQGTQQLLFSQAGPANMSAQAKNWLPGPCYRQQRVSTTLSQNNNSNFAWTGA |
| | | TKYHLNGRDSLVNPGVAMATHKDDEERFFPSSGVLMEGKQGAGRINVDYSSVMLTSEEEIKTT |
| | | NPVATEQYGVVADNLQQTN#TGPIVGNVNSQGALPGMVWQNRDVYLQGPIWAKIPHTDGNFHP |
| | | SPLMGGFGLKHPPPQILIKNIPVPADPPTTFSQAKLASFITQYSTGQVSVEIEWELQKENSKR |
| | | WNPEIQYTSNYYKSTNVDFAVNTEGTYSEPRPIGTRYLTRNL |
| 72 | AAV.ru74 | MAADGYLPDWLEDNLSEGIREWWDLKPGAPKPKANQQKQDNGRGLVLPGYKYLGPENGLDKGE |
| | version 1 | PVNAADAAALEHDKAYDQQLQAGDNPYLRYNHADAEFQERLQEDTSFGGNLCRAVEQAKKRVL |
| | | EPLGLVESPVKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRIN |
| | | FKLFNIQVKEVTQNEGTKTIANNLTSTIQVFTDSEYQLPYVLGS |
| | | AHQGCLPPFPADVEMIPQYGYLTLNNGSQAVGRSSFYCLEYFPSCMLRTGNNFEFSYNFEDVP |
| | | FHSSYAHSQSLDRLMNPLIDQYLYYLSRTQSTGGTAGTQQLLFSCAGPNNMSAQAKNWLPGPC |
| | | YRQQRVSTTLSQNNNSNFAWIGATKYHLNGRDSLVNPGVAMATHKDDEERFFPSSGVLMEGKQ |
| | | GAGKDNVDYSSVMITSEEETKTTNPVATEQYGVVADNIQQQN#AAPTVGAVNSQGALPGMVWQ |
| | | NRDVYLQGPIWAKIPHTDGNFHPSPLMGGFGLKHPPPQILIKNTPVPADPPTTFTKAKLASFI |
| | | TQYSTGQVSVEIEWELQKENSKRWNPEIQYTSNYYKSTNVDFAVNTEGTYSEPRPIGTRYLTR |
| | | NI |
| 73 | AAV.rh39 | MAADGYLPDWLEDNLSEGIREWWALKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVFQAKKRVL |
| | | EPLGLVEEAAKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRLS |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFEFSYTFEDVPFHSSYAHSQSLDRLMNPLI |
| | | DQYLYYLSRTQSTGGTQGTQQLLFSQAGPANMSAQAKNWLPGPCYRQQRVSTTLSQNNNSNFA |
| | | WTGATKYHLNGRDSLVNPGVAMATHKDDEERFFPSSGVLMFGKQGAGKDNVDYSSVMLTSEEE |
| | | IKTTNPVATEQYGVVADNLQQTN#TGPIVGNVNSQGALPGMVWQNRDVYLQGPIWAKIPHTDG |
| | | NFHPSPLMGGFGLKHPPPQILIKNTPVPADPPTTFSQAKLASFITQYSTGQVSVEIEWELQKE |
| | | NSKRWNPEIQYTSNYYKSTNVDFAVNTEGTYSEPRPIGTRYLTRNL |
| 74 | AAV.hu21 | MAADGYLPDWLEDTLSEGIRQWWKLKPGPPPPKPAERHKDDSRGLVLPGYKYLGPENGLDKGE |
| | | PVNEADAAALEHDKAYDRQLDSGDNPYLKYNHADAEFQERLKEDTSFGGNLGRAVFQAKKRIL |
| | | EPLGLVEEPVKTAPGKKRPVEHSPAEPDSSSGTGKAGQQPARKRLNFGQTGDADSVPDPRPLG |
| | | QPPAAPSGLGTNTMASGSGAPMADNNEGADGVGNSSGNWECDSTWMGDRVITTSTRTWALPTY |
| | | NNHLYKQISSQSGASNDNHYFGYSTPWGYFDENRFHCHESPRDWCRLINNNWGFRPKRLSFKL |
| | | FNIQVKEVTQNDGTTTIANNLTSTVQVFTDSEYQLPYVLGSAHQGCLPPFPADVEMVPQYGYL |
| | | TLNNGSQAVGRSSFYCLEYFPSQMLRTGNNFTFSYTFEDVPFHSSYAHSQSLDRLMNPLIDQY |
| | | LYYLSRTNTPSGTTTMSRLQFSQAGASDIRDQSRNWLPGPCYRQCRVSKTAADNNNSDYSWTG |
| | | ATKYHLNGRDSLVNPGPAMASHKDDEEKYFPQSGVLIFGKQDSGKTNVDIEKVMITDEEEIRT |
| | | TNPVATEQYGSVSTNLQSGN#TQAATSDVNTQGVLPGMVWQDRDVYLQGPIWAKIPHTDGHFH |
| | | PSPLMGGFGLKHPPPQILIKNTPVPANPSTTESAAKEASFITQYSTGQVSVEIEWELQKENSK |
| | | RWNPEIQYTSNYNKSVNVDFTVDINGVYSEPRPIGTRYLTRNL |
| 75 | AAV.hu12 | MAADGYLPDWLEDTLSEGIRQWWKLKPGPPPPKPAERHQDDSRGLVLPGYKYLGPENGLDKGE |
| | | PVNEADAAALEHDKAYDRQLDSGDNPYLKYNHADAEFQERLKEDTSFGGNLGRAVFQAKKRVL |
| | | EPLGLVEEPVKTAPGKKRPVEHSPVEPDSSSGTGKAGHQPARKRLNFGQTGDADSVPDPQPLG |
| | | QPPAAPTSLGSTTMATGSGAPMADNNEGADGVGNSSGNWECDSQWLGDRVITTSTRTWALPTY |
| | | NNHLYKQISSQSGASNDNHYFGYSTPWGYFDFNRFHCHFSPRDWQRLINNNWGFRPKRLNFKL |
| | | FNIQVKEVTQNDGTTTIANNLTSTVQVFTDSEYQLPYVLGSAHQGCLPPFPADVEMVPQYGYL |
| | | TINNGSQAVGRPSFYCLEYFPSQMLRTGNNFTFSYTFEDVPFHSSYAHSQSLDRLMNPLIDQY |
| | | LYYLNRTQSNSGTLQQSRLLFSQAGPTSMSLQAKNWLPGPCYRQQRLSKQANDNNNSNFPWTA |
| | | ATKYHLNGRDSLVNPGPAMASHKDDEEKFEPMHGTLIFGKQGTNANDADLEHVMITDEEEIRT |
| | | TNPVATEQYGNVSNNLQNSN#TGPTTENVNHQGALPGMVWQDRDVYLQGPIWAKIPHTDGHFH |
| | | PSPLMGGFGLKHPPPQIMIKNTPVPANPPTNFSSAKFASFITQYSTGQVSVEIEWELQKENSK |
| | | RWNPEIQYTSNYNKSVNVDFTVDINGVYSEPRPIGTRYLTRNL |
| 76 | AAV.hu26 | MAADGYLPDWLEDTLSEGIRQWWKLKPGPPPPKPAERHKDDSRGLVLPGYKYLGPENGLDKGE |
| | | PVNEADAAALEHDKAYDRQLDSGDNPYLKYNHADAEFQERLKEDTSFGGNLGRAVEQAKKRIL |
| | | EPLGLVEEPVKTAPGKKRPVEHSPAEPDSSSGTGKAGQQPARKRLNFGQTGDADSVPDPQPLG |
| | | QPPAAPSGLGTNTMASGSGAPMADNNEGADGVGNSSGNWECDSTWMGDRVITTSTRTWALPTY |
| | | NNHLYKQISSQSGASNDNHYFGYSTPWGYFDFNRFHCHFSPRDWQRLINNNWGFRPKRLSFKL |
| | | FNIQVKEVTQNDGTTTIANNLTSTVQVFTDSEYQLPYVLGSAHQGCLPPFPADVEMVPQYGYL |
| | | TLNNGSQAVGRSSFYCLEYFPSQMLRTGNNFTFSYTFEDVPFHSSYAHSQSLDRLMNPLIDQY |
| | | LYYLSRTNTPSGTTTMSRLQFSQAGASDIRDQSRNWLPGPCYRQCRVSKTAADNNNSDYSWTG |
| | | ATKYHLNGRDSLVNPGPAMASHKDDEEKYFPQSGVLIFGKQDSGKTNVDIEKVMITDEEEIRT |
| | | TNPVATEQYGSVSTNLQSGN#TQAATSDVNTQGVLPGMVWQDRDVYLQGPIWAKIPHTDGHFH |
| | | PSPLMGGFGLKHPPPQILIKNTPVPANPSTTFSAAKFASFITQYSTGQVSVEIEWELQKENSK |
| | | RWNPEIQYTSNYNKSVNVDFTVDINGVYSEPRPIGTRYLTRNL |
| 77 | AAV.hu53 | MAADGYLPDWLEDTLSEGIRQWWKLKPGPPPPKPAERHKDDSRGLVLPGYKYLGPENGLDKGE |
| | | PVNEADAAALEHDKAYDRQLDSGDNPYLKYNHADAEEQERLKEDTSEGGNLGRAVEQAKKRVL |
| | | EPLGLVEEPVKTAPGKKRPVEHSPAEPDSSSGTGKAGQQPARKRLNFGQTGDADSVPDPQPLR |
| | | QPPAAPTSLGSTTMATGSGAPMADNNEGADGVGNSSGNWECDSQWLGDRVITTSTRTWALPTY |
| | | NNHLYKQISSQSGASNDNHYFGYSTPWGYFDENRFHCHFSPRDWQRLINNNWGFRPKRLNFKL |
| | | FNIQVKEVTQNDGTTTIANNLTSTVQVFTDSEYQLPYVLGSAHQGCLPPFPADVEMVPQYGYL |
| | | TINNGSQAVGRSSFYCLEYFPSQMLRTGNNFQFSYTFEDVPFHSSYAHSQSLDRLMNPLIDQY |
| | | LYYLNRTQTASGTQQSRLLFSQAGPTSMSLQAKNWLPGPCYRQQRLSKQANDNNNSNFPWTGA |
| | | TKYYLNGRDSLVNPGPAMASHKDDEEKFFPMHGTLIEGKEGTNATNAELENVMITDEEEIRTT |
| | | NPVATEQYGYVSNNLQNSN#TAASTETVNHQGALPGMVWQDRDVYLQGPIWAKIPHTDGHFHP |
| | | SPLMGGFGLKHPPPQIMIKNIPVPANPPTNFSSAKFASFITQYSTGQVSVEIEWELQKENSKR |
| | | WNPEIQYTSNYNKSVNVDFTVDINGVYSEPRPIGTRYLTRNL |
| 78 | AAV3-3 | MAADGYLPDWLEDNLSEGIREWWALKPGVPQPKANQQHQDNRRGLVLPGYKYLGPGNGLDKGE |
| | | PVNEADAAALEHDKAYDQQLKAGDNPYLKYNHADAEFQERLQEDTSFGGNLGRAVEQAKKRIL |
| | | EPLGLVEEAAKTAPGKKGAVDQSPQEPDSSSGVGKSGKQPARKRLNFGQTGDSESVPDPQPLG |
| | | EPPAAPTSLGSNTMASGGGAPMADNNEGADGVGNSSGNWLCDSQWLGDRVITTSTRTWALPTY |
| | | NNHLYKQISSQSGASNDNHYFGYSTPWGYFDFNRFHCHFSPRDWQRLINNNWGFRPKKLSFKL |
| | | FNIQVRGVTQNDGTTTIANNLTSTVQVFTDSEYQLPYVLGSAHQGCLPPFPADVEMVPQYGYL |
| | | TINNGSQAVGRSSEYCLEYEPSQMLRTGNNFQESYTEEDVPEHSSYAHSQSLDRLMNPLIDQY |
| | | LYYLNRTQGTTSGTTNQSRLLFSQAGPQSMSLQARNWLPGPCYRQQRLSKTANDNNNSNFPWT |
| | | AASKYHLNGRDSLVNPGPAMASHKDDEEKFFPMHGNLIFGKEGTTASNAELDNVMITDEEEIR |
| | | TTNPVATEQYGTVANNLQSSN#TAPTTGTVNHQGALPGMVWQDRIVYLQGPIWAKIPHTDGHE |
| | | HPSPLMGGFGLKHPPPQIMIKNTPVPANPPTTFSPAKFASFITQYSTGQVSVEIEWELQKENS |
| | | KRWNPEIQYTSNYNKSVNVDFTVDINGVYSEPRPIGTRYLTRNL |
| 79 | AAV4-4 | MTDGYLPDWLEDNLSEGVREWWALQPGAPKPKANQQHQDNARGLVLPGYKYLGPGNGLDKGEP |
| | | VNAADAAALEHDKAYDQQLKAGDNPYLKYNHADAEFQQRLQGDTSEGGNLGRAVEQAKKRVLE |
| | | PLGLVEQAGETAPGKKRPLIESPQQPDSSTGIGKKGKQPAKKKLVFEDETGAGDGPPEGSTSG |
| | | AMSDDSEMRAAAGGAAVEGGQGADGVGNASGDWHCDSTWSEGHVTTTSTRTWVLPTYNNHLYK |
| | | RLGESLQSNTYNGFSTPWGYFDFNRFHCHFSPRDWQRLINNNWGMRPKAMRVKIFNIQVKEVT |
| | | TSNGETTVANNLTSTVQIFADSSYELPYVMDAGQEGSLPPFPNDVEMVPQYGYCGLVTGNTSQ |
| | | QQTDRNAFYCLEYFPSQMLRIGNNFEITYSFEKVPFHSMYAHSQSLDRLMNPLIDQYLWGLQS |
| | | TTTGTTLNAGTATTNFTKLRPTNFSNEKKNWLPGPSIKQQGFSKTANQNYKIPATGSDSLIKY |
| | | ETHSTLDGRWSALTPGPPMATAGPADSKESNSQLIFAGPKQNGNTATVPGTLIFTSEEELAAT |
| | | NATDTDMWGNLPGGDQSNS#NLPTVDRLTALGAVPGMVWQNRDIYYQGPIWAKIPHTDGHFHP |
| | | SPLIGGFGLKHPPPQIFIKNIPVPANPATTFSSTPVNSFITQYSTGQVSVQIDWEIQKERSKR |
| | | WNPEVQFTSNYGQCNSLLWAPDAAGKYTEPRAIGTRYLTEHL |
| 80 | AAV.ru74 | MAADGYLPDWLEDNLSEGIREWWDLKPGAPKPKANQQKQDNGRGLVLPGYKYLGPENGLDKGE |
| | version 2 | PVNAADAAALEHDKAYDQQLQAGDNPYLRYNHADAEFQERLQEDTSFGGNLGRAVEQAKKRVL |
| | | EPLGLVESPVKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRIN |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFEFSYNFEDVPFHSSYAHSQSLDRLMNPLI |
| | | DQYLYYLSRTQSTGGTAGTQQLLFSQAGPNNMSAQAKNWLPGPCYRQQRVSTTLSQNNNSNFA |
| | | WTGATKYHLNGRDSLVNPGVAMATHKDDEERFFPSSGVLMFGKQGAGKDNVDYSSVMLTSEEE |
| | | IKTTNPVATEQYGVVADNLQQQN#AAPIVGAVNSQGALPGMVWQNRDVYLQGPIWAKIPHTDG |
| | | NFHPSPLMGGFGLKHPPPQILIKNTPVPADPPTTFNQAKLASFITQYSTGQVSVEIEWELQKE |
| | | NSKRWNPEIQYTSNYYKSTNVDFAVNTEGTYSEPRPIGTRYLTRNL |
| 81 | AAV | MAADGYLPDWLEDNLSEGIREWWDLKPGAPKPKANQQKQDDGRGLVLPGYKYLGPENGLDKGE |
| | serotype | PVNAADAAALEHDKAYDQQLKAGDNPYLRYNHADAEFQERLQEDTSFGGNLGRAVFQAKKRVL |
| | hu.37 | EPLGLVEEAAKTAPGKKRPVEPSPQRSPDSSTGIGKKGQQPAKKRLNFGQTGDSESVPDPQPI |
| | | GEPPAGPSGLGSGTMAAGGGAPMADNNEGADGVGSSSGNWHCDSTWLGDRVITTSTRTWALPT |
| | | YNNHLYKQISNGTSGGSTNDNTYFGYSTPWGYFDFNRFHCHFSPRDWQRLINNNWGFRPKRLS |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFEFSYTFEDVPFHSSYAHSQSLDRLMNPLI |
| | | DQYLYYLSRTQSTGGTQGTQQLLFSQAGPANMSAQAKNWLPGPCYRQQRVSTTLSQNNNSNFA |
| | | WTGATKYHLNGRDSLVNPGVAMATHKDDEERFFPSSGVLMFGKQGAGRDNVDYSSVMLTSEEE |
| | | IKTTNPVATEQYGVVADNLQQTNTGPIVGNVNSQGALPGMVWQNRDVYLQGPIWAKIPHTDGN |
| | | FHPSPIMGGFGLKHPPPQILIKNTPVPADPPTTESQAKLASFITCYSTGQVSVEIEWELQKEN |
| | | SKRWNPEIQYTSNYYKSTNVDFAVNTEGTYSEPRPIGTRYLTRNL |
| 82 | AAV | MAADGYLPDW LEDNLSEGIR EWWDLKPGAP KPKANQQKQD DGRGLVLPGY |
| | serotype | EYLGPENGLD KGEPVNAADA AALEHDKAYD QQLKAGDNPY LRYNHADAEF |
| | rh.34 | QERLQEDTSF GGNLGRAVFQ AKKRVLEPLG LVEEGAKTAP GKKRPLESPQ |
| | | EPDSSSGIGK KGKQPAKKRL NFEEDTGAGD GPPEGSDTSA MSSDIEVRAA |
| | | PGGNAVDAGQ GSDGVGNASG DWHCDSTWSE GKVTTTSTRT WVLPTYNNHL |
| | | YLRLGTTSNS NTYNGFSTPW GYFDFNRFHC HFSPRDWQRL INNNWGLRPK |
| | | AMRVKIFNIQ VKEVTTSNGE TTVANNLTST VQIFADSSYE LPYVMDAGQE |
| | | GSLPPFPNDV FMVPQYGYCG IVTGENQNQT DRNAFYCLEY FPSQMLRTGN |
| | | NFETAYNFEK VPFHSMYAHS QSIDGIMNPL LDQYIWELQS TTSGETINQG |
| | | NAATTFGKIR SGDFAFYRKN WLPGPCVKQQ RESKTASQNY KIPASGGNAL |
| | | LKYDTHYTLN NRWSNIAPGP PMATAGPSDG DFSNAQLIFP GPSVTGNITT |
| | | SANNLIFTSE EEIAATNPRD TDMFGQIADN NQNATTAPIT GNVTAMGVLP |
| | | GMVWQNRDIY YQGPIWAKIP HADGHFHPSP LIGGEGLKHP PPQIFIKNTP |
| | | VPAYPATTFT AARVDSFITQ YSTGQVAVQI EWEIEKERSK RWNPEVQFTS |
| | | NCGNQSSMLW APLTTGKYTE PRVIGSRYLT NHL |
| 83 | AAV | MAADGYLPDW LEDTLSEGIR QWWKLKPGPP PPKPAERHKD DSRGLVLPGY |
| | serotype | KYLGPENGLD KGEPVNEADA AALEHDKAYD RQLDSGDNPY LKYNHADAEF |
| | rh.21 | QERLKEDTSF GGNLGRAVEQ AKKRILEPLG LVEEPVKTAP GKKRPVEHSP |
| | | AEPDSSSGTG KAGQQPARKR LNFGQTGDAD SVPDPRPLGQ PPAAPSGLGT |
| | | NTMASGSGAP MADNNEGADG VGNSSGNWHC DSTWMGDRVI TTSTRTWALP |
| | | TYNNHSYKQI SSQSGASNDN HYFGYSTPWG YFDENRFHCH FSPRDWQRLI |
| | | NNNWGFRPKR LSFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFT FSYTFEDVPF HSSYAHSQSL DRLMNPLIDQ YLYYLSRINT |
| | | PSGTTTMSRL QFSQAGASDI RDQSRNWLPG PCYRQQRVSK TAADNNNSDY |
| | | SWTGATKYEL NGRDSLVNPG PAMASHKDDE EKYEPQSGVL IFGKQDSGKT |
| | | NVDIEKVMIT DEEEIRTTNP SPLMGGFGLK HPPPQILIKN ATSDVNTQGV |
| | | LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY |
| | | TSNYNKSVNV DETVDINGVY SEPRPIGTRY LTRNL |
| 84 | AAV | MAADGYLPDW LEDNLSEGIR EWWDLKPGAP KPKANQQKQD DGRGIVLPGY |
| | serotype | KYLGPENGLD KGEPVNAADA AALEHDKAYD QQLKAGDNPY LRYNHADAEF |
| | rh.46 | QERLQEDTSF GGNLGRAVFQ AKKRVLEPLG LVEEGAKTAP GKKRPVEPSP |
| | | QRSPDSSTGI GXKGQQPARK RLNFGQTGDS ESVPDPQPIG EPPAAPSSVG |
| | | SGTMAAGGGA PMADNNEGAD GVGSSSGNWH CDSTWLGDRV ITTSTRTWAL |
| | | PTYNNHLYKQ ISNGTSGGST NDNTYFGYST PWGYFDFNRF HCHESPRDWQ |
| | | RLINNNWGER PARLSFKLEN IQVKEVTQNE GTKTIANNLT STIQVFTDSE |
| | | YQLPYVLGSA HQGCLPPFPA DVEMIPQYGY LTLNNGSQAV GRSSFYCLEY |
| | | FPSQMIRTGN NFSFSYTFED VPFHSSYAHS QSLDRLMNPL IDQYLYYLSR |
| | | TQSTGGTAGT QQLLFSQAGP SNMSAQARNW LPGPCYRQQR VSTTISQNNN |
| | | SNFAWTGATK YHLNGRDSLV NPGVAMATNK DDEDRFFPSS GILMFGKQGA |
| | | GKDNVDYSNV MLTSEEEIKA TNPVATEQYG VVADNLQQQN TAPIVGAVNS |
| | | QGALPGMVWQ NREVYLQGPT WAKTPHTDGN FHPSPIMGGF GIKHPPPQIL |
| | | IKNTPVPADP PTAFNQAKLN SFITQYSTGQ VSVEIEWELQ KENSKRANPE |
| | | IQYTSNYYKS TNVDFAVNTE GVYSEPRPIG TRYLTRNL |
| 87 | AAV3B | MAADGYLPDWLEDNLSEGIREWWALKPGVPQPKANQQHQDNRRGLVLPGYKYLGPGNGLDKGE |
| | | PVNEADAAALEHDKAYDQQLKAGDNPYLKYNHADAEFQERLQEDTSFGGNLGRAVEQAKKRIL |
| | | EPLGLVEEAAKTAPGKKRPVDQSPQEPDSSSGVGKSGKQPARKRLNFGQTGDSESVPDPQPLG |
| | | EPPAAPTSLGSNTMASGGGAPMADNNEGADGVGNSSGNWECDSQWLGDRVITTSTRTWALPTY |
| | | NNHLYKQISSQSGASNDNHYFGYSTPWGYFDFNRFHCHESPRDWCRLINNNWGFRPKKLSFKL |
| | | FNIQVKEVTQNDGTTTIANNLTSTVQVFTDSEYQLPYVLGSAHQGCLPPFPADVEMVPQYGYL |
| | | TINNGSQAVGRSSFYCLEYFPSQMLRTGNNFQFSYTFEDVPFHSSYAHSQSLDRLMNPLIDQY |
| | | LYYLNRTQGTTSGTTNQSRLLFSQAGPQSMSLQARNWLPGPCYRQQRLSKTANDNNNSNFPWT |
| | | AASKYHLNGRDSLVNPGPAMASHKDDEEKFFPMHGNLIFGKEGTTASNAELDNVMITDEEEIR |
| | | TTNPVATEQYGTVANNLQSSN#TAPTTRTVNDQGALPGMVWQDRIVYLQGPIWAKIPHTDGHF |
| | | HPSPLMGGFGLKHPPPQIMIKNTPVPANPPTTESPAKFASFITQYSTGQVSVEIEWELQKENS |
| | | KRWNPEIQYTSNYNKSVNVDFTVDTNGVYSEPRPIGTRYLTRNL |
| 88 | AAV.rh73 | MAADGYLPDWLEDNLSEGIREWWDLKPGAPKPKANQQKQDDGRGLVLPGYKYLGPENGLDKGE |
| | | PVNAADAAALEHDKAYDQQLKAGDNPYLRYNHADAEFQERLQEDTSFGGNLGRAVEQAKKRVL |
| | | EPLGLVEEGAKTAPGKKRPVEPSPQRSPDSSTGIGKKGQQPARKRLNFGQTGDSESVPDPQPL |
| | | GEPPAAPSSVGSGTMAAGGGAPMADNNEGADGVGSSSGNWHCDSTWLGDRVITTSTRTWALPT |
| | | YNNHLYKQTSNGTSGGSTNDNTYFGYSTPWGYFDFNRFHCHFSPRDWQRLINNNWGFRPKRIS |
| | | FKLFNIQVKEVTQNEGTKTIANNLTSTIQVFTDSEYQLPYVLGSAHQGCLPPFPADVEMIPQY |
| | | GYLTLNNGSQAVGRSSFYCLEYFPSQMLRTGNNFSFSYTFEDVPFHSSYAHSQSLQRLMNPLI |
| | | DQYLYYLSRTQSTGGTAGTQQLLFSQAGPSNMSAQARNWLPGPCYRQQRVSTTLSQNNNSNFA |
| | | WTGATKYHLNGRDSLVNPGVAMATNKDDEDRFFPSSGILMFGKQGAGKDNVDYSNVMLTSEEE |
| | | IKTTNPVATEQYGVVADNLQRQN#TAPIVGAVNSQGALPGMVWQNRDVYLQGPIWAKIPHTDG |
| | | NFHPSPLMGGFGLKHPPPQILIKNTPVPADPPTAFNQAKLNSFITQYSTGQVSVEIEWELQKE |
| | | NSKRWNPEIQYTSNYYKSTNVDFAVNTEGVYSEPRPIGTRYLTRNL |
| 89 | AAV.hu51 | MAADGYLPDWLEDTLSEGIRQWWKLKPGPPPPKPAERHKDDSRGLVLPGYKYLGPENGLDKGE |
| | | PVNEADAAALEHDKAYDRQLDSGDNPYLKYNHADAEFQERLKEDTSFGGNLGRAVEQAKKRVL |
| | | EPLGLVGEPVKTAPGKKRPVEHSPVEPDSSSGTGKAGQQPARKRLNFGQTGDADSVPDPQPLG |
| | | QPPAAPSGLGTNTMATGSGAPMADNNEGADGVGNSSGNWECDSTWMGDRDSVPDPQPLGQPPA |
| | | APSGLGTNTMATGSGAPMADNNEGADGVGNSSGNWHCDSTWMGDRVITTSTRTWALPTYNNHL |
| | | YKQISSQSGASNDNHYFGYSTPWGYFDFNRFHCHFSPRDWQRLINNNWGFRPKRLNEKLENIQ |
| | | VKEVTQNDGTTTIANNLTSTVQVFTDSEYQLPYVLGSAHQGCLPPFPADVEMVPQYGYLTLNN |
| | | GSQAVGRSSFYCLEYFPSQMLRTGNNFTFSYTFEDVPFHSGYAHSQSLDRLMNPLIDQYLYYL |
| | | STTNTPSGTTTQSRLQFSQAGASDIRDQSRNWLPGPCYRQQRVSKTSADNNNSEYSWTGATKY |
| | | HLNGRDSTVNPGPAMASHKDNEEKFFPQSGVLIFGKQGSEKTNVETEKVMTTDEEETRTTNPV |
| | | ATEQYGSVSTNLQRGN#RQAATADVNTQGVLPGMVWQDRDVYLQGPIWAKIPHTDGHFHPSPL |
| | | MGGFGLKHEPPPQILIKNTPVPANPSTTFSAAKFASEITQYSTGQVSVEIEWELQKENSKRWN |
| | | PEIQYTSNYNKSVNVDFTVDINGVYSEPRPIGTRYLTRNL |

TABLE 10
Peptide Inserts

| SEQ ID NO | Amino Acid Sequence |
|---|---|
| 1 | TLAAPFK |
| 2 | DDDDDDDD |
| 3 | LSSRLDA |
| 4 | CLSSRLDAC |
| 5 | CLPVASC |
| 6 | LPVAS |
| 7 | ASSLNIA |
| 9 | TQT/STQT |
| 10 | HAIYPRH |
| 12 | RTIGPSV |
| 13 | CRTIGPSVC |
| 14 | SITL VKSTQTV |
| 15 | TILSRSTQTG |
| 16 | QAVRTSL |
| 17 | QAVRTSH |
| 20 | TLAVPFK |
| 126 | VQVGRTS |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises:
culturing a cell comprising one or more polynucleotides, wherein the one or more polynucleotides comprise:
(a) one or more polynucleotides encoding VP1, VP2 and VP3 proteins; wherein at least one polynucleotide encodes a VP3-DARPin protein, which comprises a VP3 protein having a DARPin inserted within VR-IV or VR-VIII of the VP3 protein:
(b) a polynucleotide encoding afunctional rep gene;
(c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and
(d) one or more polynucleotides encoding sufficient helper functions to permit packaging of the artificial genome into the AAV capsid protein under conditions which permit packaging of the genome into the AAV capsid:
wherein the cell is cultured under conditions that allow production of the recombinant AAV (rAAV) particle.

2. The method of claim 1, wherein (a) comprises: (i) a polynucleotide encoding wild-type or parental VP1, VP2 and VP3 proteins and (ii) a polynucleotide encoding VP3-DARPin protein.

3. The method of claim 1, wherein (a) comprises: (i) a polynucleotide encoding wild-type or parental VP1 and VP2 proteins, wherein the start codon for VP3 is mutated, and (ii) a polynucleotide encoding VP3-DARPin protein

4. The method of claim 2 or claim 3, wherein (i) the polynucleotide encoding wild-type or parental VP1, VP2 and VP3 proteins or the polynucleotide encoding wild-type or parental VP1 and VP2 proteins and (ii) the polynucleotide encoding functional rep gene are operably linked.

5. The method of any one of claims 2 to 4, wherein prior to the step of culturing the cell comprising one or more polynucleotides, (i) the polynucleotide encoding wild-type or parental VP1, VP2 and VP3 proteins or the polynucleotide encoding wild-type or parental VP1 and VP2 proteins and (ii) the polynucleotide encoding VP3-DARPin protein were introduced into the cell on separate plasmids.

6. The method of claim 5, wherein prior to the step of culturing a cell comprising one or more polynucleotides, (i) the polynucleotide encoding wild-type or parental VP1, VP2 and VP3 proteins or the polynucleotide encoding wild-type or parental VP1 and VP2 proteins and (ii) the polynucleotide encoding VP3-DARPin protein were introduced into the cell at a ratio of about 50:50, about 70:30, about 80:20 or about 90:10.

7. The method of any one of claims 1 to 6, wherein the DARPin is inserted into the VP3 protein at VR-IV.

8. The method of any one of claims 1 to 7, wherein the DARPin is flanked on either one or both ends by a linker.

9. The method of claim 8, wherein the linker is a GS linker or a PT-linker.

10. The method of any one of claims 1 to 9, wherein the polynucleotide encoding VP3-DARPin protein encodes SEQ ID NO: 132 or SEQ ID NO: 133.

11. An engineered rAAV particle made by the method of any one of claims 1 to 10.

12. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises:
culturing a cell comprising one or more polynucleotides, wherein the one or more polynucleotides comprise:
(a) one or more polynucleotides encoding VP1, VP2 and VP3 proteins; wherein at least one polynucleotide encodes a VP1-DARPin protein, which comprises a VP1 protein having a DARPin inserted within VR-IV or VR-VIII of the VP1 protein:
(b) a polynucleotide encoding afunctional rep gene;
(c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and
(d) one or more polynucleotides encoding sufficient helper functions to permit packaging of the artificial genome into the AAV capsid protein under conditions which permit packaging of the genome into the AAV capsid; wherein the cell is cultured under conditions that allow production of the recombinant AAV (rAAV) particle.

13. The method of claim 12, wherein (a) comprises: (i) a polynucleotide encoding wild-type or parental VP1, VP2 and VP3 proteins and (ii) a polynucleotide encoding VP1-DARPin protein.

14. The method of claim 12, wherein (a) comprises: (i) a polynucleotide encoding wild-type or parental VP2 and VP3 proteins, wherein the start codon for VP1 is mutated, and (ii) a polynucleotide encoding VP1-DARPin protein.

15. The method of claim 13 or claim 14, wherein (i) the polynucleotide encoding wild-type or parental VP1, VP2 and VP3 proteins or the polynucleotide encoding wild-type or parental VP2 and VP3 proteins and (ii) the polynucleotide encoding functional rep gene are operably linked.

16. The method of any one of claims 13 to 15, wherein prior to the step of culturing the cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein were introduced into the cell on separate plasmids.

17. The method of claim 16, wherein prior to the step of culturing a cell comprising one or more polynucleotides, (i) the polynucleotide encoding wild-type or parental VP1, VP2 and VP3 proteins or the polynucleotide encoding wild-type or parental VP2 and VP3 proteins and (ii) the polynucleotide encoding VP1-DARPin protein are introduced into the cell at a ratio of about 50:50, about 70:30, about 80:20 or about 90:10.

18. The method of any one of claims 12 to 17, wherein the DARPin is inserted into the VP1 protein at VR-IV.

19. The method of any one of claims 12 to 18, wherein the DARPin is flanked on either one or both ends by a linker.

20. The method of claim 19, wherein the linker is a GS linker or PT linker.

21. The method of any one of claims 12 to 20, wherein the polynucleotide encoding VP1-DARPin protein encodes SEQ ID NO: 114 or SEQ ID NO: 115.

22. An engineered rAAV particle made by the method of any one of claims 12 to 21.

23. A method of producing a recombinant AAV (rAAV) particle, wherein the rAAV particle comprises a capsid and an artificial genome, wherein the capsid comprises at least one rAAV capsid protein comprising a DARPin which is displayed on the surface of the capsid, wherein the method comprises:
culturing a cell comprising one or more polynucleotides, wherein the one or more polynucleotides comprise:
(a) one or more polynucleotides encoding VP1, VP2 and VP3 proteins; wherein at least one polynucleotide encodes a VP2-DARPin protein, which comprises a VP2 protein having a DARPin inserted within or at the VR-IV or VR-VIII of the VP2 protein;
(b) a polynucleotide encoding afunctional rep gene;
(c) a polynucleotide comprising the artificial genome comprising at least one AAV inverted terminal repeat (ITR) and a non-AAV nucleic acid sequence encoding a gene product operably linked to a regulatory control element which directs expression of the gene product in a target cell; and
(d) one or more polynucleotides encoding sufficient helper functions to permit packaging of the artificial genome into the AAV capsid protein under conditions which permit packaging of the genome into the AAV capsid;
wherein the cell is cultured under conditions that allow production of the recombinant AAV (rAAV) particle.

24. The method of claim 23, wherein (a) comprises: (i) a polynucleotide encoding wild-type or parental VP1, VP2 and VP3 proteins and (ii) a polynucleotide encoding VP2-DARPin protein.

25. The method of claim 23, wherein (a) comprises: (i) a polynucleotide encoding wild-type or parental VP1 and VP3 proteins and (ii) a polynucleotide encoding VP2-DARPin protein.

26. The method of claim 24 or claim 25, wherein (i) the polynucleotide encoding wild-type or parental VP1, VP2 and VP3 proteins or the polynucleotide encoding wild-type or parental VP1 and VP3 proteins and (ii) the polynucleotide encoding functional rep gene are operably linked.

27. The method of any one of claims 24 to 26, wherein prior to the step of culturing the cell comprising one or more polynucleotides, (i) the polynucleotide encoding wild-type or parental VP1, VP2 and VP3 proteins or the polynucleotide encoding wild-type or parental VP1 and VP3 proteins and (ii) the polynucleotide encoding VP2-DARPin protein were introduced into the cell on separate plasmids.

28. The method of claim 27, wherein prior to the step of culturing a cell comprising one or more polynucleotides, (i) the polynucleotide encoding wild-type or parental VP1, VP2 and VP3 proteins or the polynucleotide encoding wild-type or parental VP1 and VP3 proteins and (ii) the polynucleotide encoding VP2-DARPin protein are introduced into the cell at a ratio of about 50:50, about 70:30, about 80:20 or about 90:10.

29. The method of any one of claims 23 to 28, wherein the DARPin is inserted into the VP2 protein at VR4.

30. The method of any one of claims 23 to 29, wherein the DARPin is flanked on either one or both ends by a linker.

31. The method of claim 30, wherein the linker is a GS linker or a PT linker.

32. The method of any one of claims 23 to 31, wherein the polynucleotide encoding VP1-DARPin protein encodes SEQ ID NO: 119.

33. An engineered rAAV particle made by the method of any one of claims 23 to 32.

34. A recombinant AAV capsid protein comprising an insertion of a DARPin which targets a cell surface molecule and wherein the DARPin is flanked on at least one end by a linker, wherein a recombinant AAV particle incorporating the recombinant AAV capsid protein has cell transduction activity.

35. The recombinant AAV capsid protein of claim 34, wherein the AAV capsid protein is an AAV9 or AAVhu32 capsid protein and comprises no other substitutions or insertions.

36. The recombinant AAV capsid protein of claim 34, which further comprises one or more amino acid substitutions and/or insertions relative to the wild type or unengineered capsid protein which when incorporated into an rAAV capsid exhibits reduced transduction or exhibits increased transduction of at least one tissue type relative to an rAAV capsid incorporating the wild type or unengineered capsid protein.

37. The recombinant AAV capsid protein of claim 36, in which the rAAV capsid protein has (1) a G266A substitution, a N272A substitution, a W503A substitution or a VQVGRTS insertion between 454 and 455 or (2) 496-NNN/AAA-498 substitutions, or (3) a combination thereof, for an AAV9 capsid protein, or corresponding substitutions in a capsid protein of another AAV type capsid.

38. The recombinant AAV capsid protein of claim 37 which comprises N272A and 496-NNN/AAA-498 substitutions.

39. The recombinant AAV capsid protein of any one of claims 34 to 38 wherein the insertion of the DARPin is near the VP2 initiation codon or within the VR-1 region, VR-IV region, or VR-VIII region of the VP2 protein.

40. The recombinant AAV capsid protein of any one of claims 34 to 39, wherein the insertion is at one of positions 138, 262-273, 452-461, or 585-593, or replaces one or more of amino acids 452-461 or 585-593, for AAV9 as numbered for the VP1 amino acid sequence or corresponding position for a different AAV capsid.

41. The recombinant AAV capsid protein of any one of claims 34 to 40, wherein the insertion is between Q588 and A589, S268 and S269, before or after 1451, N452, G453, S454, G455, Q456, N457, Q458, Q459, T460, or L461 of AAV9 as numbered for the VP1 amino acid sequence or corresponding position of a different AAV capsid protein.

42. The recombinant AAV capsid protein of any one of claims 34 to 41 wherein the DARPin replaces one or more of amino acids 452-461 or 585-593 of AAV9 as numbered for the VP1 amino acid sequence or corresponding amino acids of a different AAV capsid protein.

43. The recombinant AAV capsid protein of any one of claims 34 to 42 wherein the linker comprises a (G)ₙS linker, where n=2-10.

44. The recombinant AAV capsid protein of any one of claims 34 to 43, wherein the linker comprises GGS, GGGGS (SEQ ID NO: 127), 4GSx2 (SEQ ID NO: 135), 4GSx3 (SEQ ID NO: 136), 4GSx4 (SEQ ID NO: 137), GS-4GSx4-GS (SEQ ID NO: 138), GS-PT linker-GS (SEQ ID NO: 139) or GS-PT linker-GS (SEQ ID NO: 140) and is at both the N-terminus and C-terminus of the DARPin insert.

45. The recombinant AAV capsid protein of any one of claims 34 to 44 where the linker comprises a PT linker.

46. The recombinant AAV capsid protein of any one of claims 34 to 45 wherein the DARPin targets a CNS cell surface protein.

47. The recombinant AAV capsid protein of any one of claims 34 to 46, which when incorporated into a rAAV particle, the rAAV particle has increased targeting, binding or transduction into CNS cells, relative to a rAAV particle incorporating the corresponding capsid protein without the DARPin insertion.

48. The recombinant AAV capsid protein of any one of claims 34 to 47 wherein the DARPin targets a human receptor or GluA4.

49. The recombinant AAV capsid protein of claim 34 which has an amino acid sequence of SEQ ID NO: 113, 114 or 115.

50. The recombinant AAV capsid protein of claim 34 or claim 49 which is encoded by the nucleic acid sequences of SEQ ID NOs: 101/103 or 105/107 or 120.

51. The recombinant AAV capsid protein of any one of claims 34 to 50 which is a VP1 or VP2 or VP3 capsid protein.

52. A nucleic acid comprising a nucleotide sequence encoding the rAAV capsid protein of any one of claims 34 to 51, or encoding an amino acid sequence sharing at least 80% identity therewith and retaining biological activity of the rAAV capsid protein, optionally wherein the nucleotide sequence encoding the rAAV capsid protein is operably linked to a promoter and a polyadenylation sequence.

53. The nucleic acid of claim 52 encoding the rAAV capsid protein of any one of claims 28 to 45.

54. A plasmid vector comprising the nucleic acid of claim 52 or claim 53, which is replicable in a bacterial cell.

55. A bacterial host cell comprising the plasmid vector of claim 54.

56. A packaging cell which expresses the nucleic acid of claim 52 or claim 53 to produce AAV particles comprising the capsid protein encoded by said nucleotide sequence.

57. The packaging cell of claim 56, wherein the nucleic acid encodes a recombinant VP2 capsid protein and the packaging cell further comprises a nucleic acid which expresses AAV VP1 and VP3 capsid proteins, optionally having a mutated VP2 start codon.

58. The packaging cell of claim 56 wherein the nucleic acid encodes a recombinant VP1 capsid protein and the packaging cell further comprises a nucleic acid which expresses AAV VP2 and VP3 capsid proteins, optionally having a mutated VP1 start codon.

59. An rAAV particle comprising the rAAV capsid protein of any one of claims 34 to 51.

60. The rAAV particle of claim 59, wherein the insertion of the DARPin is (1) in the recombinant VP1 capsid protein but not the VP2 or VP3 capsid protein; (2) in the recombinant VP2 capsid but not in the VP1 or VP3 capsid protein or (3) in the recombinant VP3 capsid but not in the VP1 or VP2 capsid protein.

61. The rAAV particle of claim 59 or claim 60 further comprising a nucleic acid comprising a transgene encoding a therapeutic protein or a therapeutic nucleic acid operably linked to a regulatory sequence for expression of the therapeutic protein or the therapeutic nucleic acid in the target cells or tissue, wherein the transgene and regulatory sequence are flanked by AAV ITR sequences.

62. The rAAV particle of claim 61, wherein the regulatory sequence promotes expression of the therapeutic protein or therapeutic nucleic acid in muscle or CNS cells.

63. A pharmaceutical composition comprising the rAAV particle of any one of claims 59 to 62 and a pharmaceutically acceptable carrier.

64. A method of delivering a transgene to a cell, said method comprising contacting said cell with the rAAV particle of any of claims 59 to 62 wherein said transgene is delivered to said cell.

65. The method of claim 64 in which the cell is a CNS cell, cardiac muscle cell or skeletal muscle cell.

66. A method of delivering a transgene to a target tissue of a subject having a disease associated with the target tissue and treatable by expression of said transgene in said tissue and in need treatment, said method comprising administering to said subject the rAAV particle of any one of claims 59 to 62, wherein the transgene is delivered to and expressed in said target tissue.

67. The method of claim 66 wherein the transgene is a muscle disease or heart disease therapeutic and said target tissue is cardiac muscle or skeletal muscle.

68. The method of claim 66 or claim 67, wherein the rAAV is administered systemically, including intravenously or intramuscularly.

69. The method of any one of claims 66 to 68, wherein the transgene is a CNS disease therapeutic and said target tissue is CNS.

70. The method of claim 69 wherein the rAAV is administered intrathecally, intracerebroventricularly or intravenously.

71. A pharmaceutical composition for use in delivering a transgene to a cell, said pharmaceutical composition comprising the rAAV particle of any of claims 59 to 62, wherein said transgene is delivered to said cell.

72. A pharmaceutical composition for use in delivering a transgene encoding a therapeutic protein or therapeutic nucleic acid to a target tissue of a subject having a disease associated with the target tissue and in need treatment, said pharmaceutical composition comprising the rAAV particle of any of claims 59 to 62, wherein the transgene is delivered to said target tissue.

73. A host cell comprising:
(a) an artificial genome comprising an expression cassette flanked by AAV inverted terminal repeats (ITRs), wherein the expression cassette comprises a transgene encoding a therapeutic protein operably linked to a regulatory element that promotes transgene expression in target cells;
(b) a trans expression cassette lacking AAV ITRs, wherein the trans expression cassette encodes an AAV rep protein and one or more of wild-type or parental VP1, VP2 and VP3 operably linked to expression control elements that drive expression of the AAV rep protein and AAV capsid protein in the host cell in culture and supply the rep and capsid proteins in trans;
(c) a secondary expression cassette lacking AAV ITRs, wherein the secondary expression cassette encodes the recombinant AAV capsid protein of any of claims 34 to 51; and
(d) sufficient adenovirus helper functions to permit replication and packaging of the artificial genome by the AAV capsid proteins.

74. A method of producing recombinant AAVs comprising:
(a) culturing a host cell containing:
(i) an artificial genome comprising an expression cassette flanked by AAV inverted terminal repeats (ITRs), wherein the expression cassette comprises a transgene encoding a therapeutic protein operably linked to a regulatory element that promotes transgene expression in target cells;
(ii) a trans expression cassette lacking AAV ITRs, wherein the trans expression cassette encodes an AAV rep protein and one or more of wild-type or parental VP1, VP2 and VP3 operably linked to expression control elements that drive expression of the AAV rep protein and AAV capsid protein in the host cell in culture and supply the rep and cap proteins in trans;
(iii) a secondary expression cassette lacking AAV ITRs, wherein the secondary expression cassette encodes the recombinant AAV capsid protein of any one of claims 34 to 51; and
(iv) sufficient adenovirus helper functions to permit replication and packaging of the artificial genome by the AAV capsid proteins; and
(b) recovering recombinant AAV encapsidating the artificial genome from the cell culture.
